(19) United States
(12) Patent Application Publication
(10) Pub. No.: US 2003/0143539 A1
(43) Pub. Date: Jul. 31, 2003

(54) Gene expression profiling of primary breast carcinomas using arrays of candidate genes

(76) Inventors: Francois Bertucci, Marseille (FR); Remi Houlgatte, Marseille (FR); Daniel Birnbaum, Marseille (FR); Catherine Nguyen, Marseille (FR); Patrice Viens, Marseille (FR); Vincent Fert, Allauch (FR)

Correspondence Address: Schnader Harrison Segal & Lewis, IP Department, 36th Floor, 1600 Market Street, Philadelphia, PA, 19103, US

(21) Appl. No.: 10/007,926

(22) Filed: Dec. 7, 2001

Related U.S. Application Data
(60) Provisional application No. 60/254,090, filed on Dec. 8, 2000.

(51) Int. Cl. C12Q 1/68; C07H 21/04

(52) U.S. Cl. 435/6; 536/23.1

(57) ABSTRACT

A polynucleotide library useful in the molecular characterization of a carcinoma, the library including a pool of polynucleotide sequences or subsequences thereof wherein the sequences or subsequences are overexpressed in tumor cells, further wherein the sequences or subsequences correspond substantially to any of the polynucleotide sequences set forth in any of SEQ ID NOS: 1-468 or the complement thereof.

### CROSS-REFERENCE TO RELATED APPLICATION

This application claims the benefit of priority of provisional application Serial No. 60/254,090 filed Dec. 8, 2000.

### FIELD OF THE INVENTION

This invention relates to polynucleotide analysis and, in particular, to polynucleotide expression profiling of carcinomas using arrays of candidate polynucleotides.

### BACKGROUND

Pathologists and clinicians in charge of the management of breast cancer patients are facing two major problems, namely the extensive heterogeneity of the disease and the lack of factors—among conventional histological and clinical features—predicting with reliability the evolution of the disease and its sensitivity to cancer therapies. Breast tumors of the same apparent prognostic type vary widely in their responsiveness to therapy and consequent survival of the patient. New prognostic and predictive factors are needed to allow an individualization of therapy for each patient.

Great hope is currently being placed on molecular studies, which address the problem in a global fashion. Methods such as cytogenetics, comparative genomic hybridization, and whole-genome allelotyping have addressed the issue at the genome level. Currently, the modifications that take place in human tumors at the level of transcription can also be studied in a large, unprecedented scale, using new methods such as cDNA arrays that allow quantitative measurement of the mRNA expression levels of many genes simultaneously. Thus, it would be advantageous to provide a means to assess the capacity of cDNA array testing-in clinical practice to better classify an heterogeneous cancer into tumor subtypes with more homogeneous clinical outcomes, and to identify new potential prognostic factors and therapeutics targets.

### SUMMARY OF THE INVENTION

The invention relates to a polynucleotide library useful in the molecular characterization of a carcinoma, the library including a pool of polynucleotide sequences or subsequences thereof wherein the sequences or subsequences are either underexpressed or overexpressed in tumor cells, further wherein the sequences or subsequences correspond substantially to any of the polynucleotide sequences set forth in any of SEQ ID NOS: 1-468 or the complement thereof.

### BRIEF DESCRIPTION OF THE DRAWINGS

FIG. 1 shows an example of differential gene expression between normal breast tissue (NB) and breast tumor samples.

FIG. 2 is a representation of expression levels of 176 genes in normal breast tissue (NB) and 34 samples of breast carcinoma.

FIG. 3 is prognostic classification of breast cancer by gene expression profiling.

FIG. 4 shows the correlation of GATA3 (SEQ ID NO: 78) expression with ER phenotype.

### DETAILED DESCRIPTION OF THE INVENTION

In the context of this disclosure, a number of terms shall be utilized.

The term “polynucleotide” refers to a polymer of RNA or DNA that is single-stranded, optionally containing synthetic, non-natural or altered nucleotide bases. A polynucleotide in the form of a polymer of DNA may be comprised of one or more segments of cDNA, genomic DNA or synthetic DNA.

The term “subsequence” refers to a sequence of nucleic acids that comprises a part of a longer sequence of nucleic acids.

The term “immobilized on a support” means bound directly or indirectly thereto including attachment by covalent binding, hydrogen bonding, ionic interaction, hydrophobic interaction or otherwise.

Breast cancer is characterized by an important histoclinical heterogeneity that currently hampers the selection of the most appropriate treatment for each case. This problem could be solved by the identification of new parameters that better predict the natural history of the disease and its sensitivity to treatment. An important object of the present invention relates to a large-scale molecular characterization of breast cancer that could help in prediction, prognosis and cancer treatment.

An important aspect of the invention relates to the use of cDNA arrays, which allows quantitative study of mRNA expression levels of 188 candidate genes in 34 consecutive primary breast carcinomas in three areas: comparison of tumor samples, correlations of molecular data with conventional histoclinical prognostic features and gene correlations. The experimentation evidenced extensive heterogeneity of breast tumors at the transcriptional level. Hierarchical clustering algorithm identified two molecularly distinct subgroups of tumors characterized by a different clinical outcome after chemotherapy. This outcome could not have been predicted by the commonly used histoclinical parameters. No correlation was found with the age of patients, tumor size, histological type and grade. However, expression of genes was differential in tumors with lymph node metastasis and according to the estrogen receptor status; ERBB2 (SEQ ID No: 119) expression was strongly correlated with the lymph node status (p≦0.0001) and that of GATA3 (SEQ ID No: 78) with the presence of estrogen receptors (p≦0.001). Thus, experimental results identified new ways to group tumors according to outcome and new potential targets of carcinogenesis. They show that the systematic use of cDNA array testing holds great promise to improve the classification of breast cancer in terms of prognosis and chemosensitivity and to provide new potential therapeutic targets.

DNA arrays consist of large numbers of DNA molecules spotted in a systematic order on a solid support or substrate such as a nylon membrane, glass slide, glass beads, a membrane on a glass support, or a silicon chip. Depending on the size of each DNA spot on the array, DNA arrays can be categorized as microarrays (each DNA spot has a diameter less than 250 microns) and macroarrays (spot diameter is greater than 300 microns). When the solid substrate used is small in size, arrays are also referred to as DNA chips. Depending on the spotting technique used, the number of spots on a glass microarray can range from hundreds to thousands.

DNA microarrays serve a variety of purposes, including gene expression profiling, de novo gene sequencing, gene mutation analysis, gene mapping and genotyping. cDNA microarrays are printed with distinct cDNA clones isolated from cDNA libraries. Therefore, each spot represents an expressed gene, since it is derived from a distinct mRNA.

Typically, a method of monitoring gene expression involves (1) providing a pool of sample polynucleotides comprising RNA transcript(s) of one or more target gene(s) or nucleic acids derived from the RNA transcript(s); (2) reacting, such as hybridizing the sample polynucleotide to an array of probes (for example, polynucleotides obtained from a polynucleotide library) (including control probes) and (3) detecting the reacted/hybridized polynucleotides. Detection can also involve calculating/quantifying a relative expression (transcription) level.

The present invention concerns a polynucleotide library useful in the molecular characterization of a carcinoma, said library comprising a pool of polynucleotide sequences or subsequences thereof wherein said sequences or subsequences are either underexpressed or overexpressed in tumor cells, flrher wherein said sequences or subsequences correspond substantially to any of the polynucleotide sequences set forth in any of SEQ ID Nos: 1-468 in annex or the complement thereof.

Obviously, sequences having a great degree of homology with the above sequences could also be used to realize the molecular characterization of the invention, namely when those sequences present one or a few punctual mutations when compared with any one of the sequences represented by SEQ ID Nos: 1-468.

A particular embodiment of the invention relates to a polynucleotide library of sequences or subsequences corresponding substantially to any combination of at least one polynucleotide sequence selected among those included in each one of predefined polynucleotide sequence sets 1 to 188 as defined in table 4.

A polynucleotide sequence library useful for the realization of the invention can comprise also any sequence comprised between 3′ end and 5′ end of each polynucleotide sequence set as defined in table 4, allowing the complete detection of the implicated gene.

The invention relates also to a polynucleotide library useful to differentiate a normal cell from a cancer cell wherein the pool of polynucleotide sequences or subsequences correspond substantially to any combination of at least one polynucleotide sequence selected among those included in each one of predefined polynucleotide sequences sets indicated in table 5, useful in differentiating a normal cell from a cancer cell.

Preferably the polynucleotide library useful to differentiate a normal cell from a cancer cell corresponds substantially to any combination of at least one polynucleotide sequence selected among those included in each one of predefined polynucleotide sequence sets indicated in table 5A, and of at least one polynucleotide sequence selected among those included in each one of predefined polynucleotide sequence sets indicated in table 5B.

The detection of an overexpression of genes identified with sets of polynucleotide sequences defined in table 5A, together with detection of an underexpression of genes identified with sets of polynucleotide sequences defined in table 5B allows distinction between normal patients and patients suffering from tumor pathology.

The invention relates also to a polynucleotide library useful to detect a hormone-sensitive tumor cell wherein the pool of polynucleotide sequences or subsequences correspond substantially to any combination of at least one polynucleotide sequence selected among those included in each one of predefined polynucleotide sequence sets defined in table 6.

Preferably the polynucleotide library useful to detect a hormone-sensitive tumor cell correspond substantially to any combination of at least one polynucleotide sequence selected among those included in each one of predefined polynucleotide sequence sets defined in table 6A together with at least one polynucleotide sequence selected among those included in each one of predefined polynucleotide sequence sets defined in table 6B.

The detection of an overexpression of genes identified with sets of polynucleotides sequences defined in table 6A, together with detection of an underexpression of genes identified with sets of polynucleotides sequences defined in table 6B allows distinction between patients having a hormone-sensitive tumor and patients having a hormone-resistant tumor.

The invention also concerns a polynucleotide library useful to differentiate a tumor in which a lymph node has been invaded by a tumor cell from a tumor in which a lymph node has not been invaded by a tumor cell wherein the pool of polynucleotide sequences or subsequences correspond substantially to any combination of at least one polynucleotide sequence selected among those included in each one of predefined polynucleotide sequence sets defined in table 7.

Preferably, the polynucleotide library useful to differentiate a tumor in which a lymph node has been invaded by a tumor cell from a tumor in which a lymph node has not been invaded by a tumor cell correspond substantially to any combination of at least one polynucleotide sequence selected among those included in each one of predefined polynucleotide sequence sets defined in table 7A together with at least one polynucleotide sequence selected among those included in each one of predefined polynucleotide sequence sets defined in table 7B.

The detection of an overexpression of genes identified with sets of polynucleotide sequences defined in table 7A, together with detection of an underexpression of genes identified with sets of polynucleotide sequences defined in table 7B allows distinction between patients having a tumor in which a lymph node has been invaded by a tumor cell and patients having a tumor in which a lymph node has not been invaded by a tumor cell.

The invention concerns also a polynucleotide library useful to differentiate anthracycline-sensitive tumors from anthracycline-insensitive tumors wherein the pool of polynucleotide sequences or subsequences correspond substantially to any combination of at least one polynucleotide sequence selected among those included in each one of predefined polynucleotide sequence sets defined in table 8.

Preferably, the polynucleotide library useful to differentiate anthracycline-sensitive tumors from anthracycline-insensitive tumors correspond substantially to any combination of at least one polynucleotide sequence selected among those included in each one of predefined polynucleotide sequence sets defined in table 8A together with at least one polynucleotide sequence selected among those included in each one of predefined polynucleotide sequence sets defined in table 8B.

The detection of an overexpression of genes identified with sets of polynucleotide sequences defined in table 8A, together with detection of an underexpression of genes identified with sets of polynucleotide sequences defined in table 8B allows distinction between patients having an anthracycline-sensitive tumor from patients having an anthracycline-insensitive tumor.

The invention also concerns a polynucleotide library useful to classify good and poor prognosis primary breast tumors wherein the pool of polynucleotide sequences or subsequences correspond substantially to any combination of at least one polynucleotide sequence selected among those included in each one of predefined polynucleotide sequence sets defined in table 9.

Preferably, the polynucleotide library useful to classify good and poor prognosis primary breast tumors correspond substantially to any combination of at least one polynucleotide sequence selected among those included in each one of predefined polynucleotide sequence sets defined in table 9A together with at least one polynucleotide sequence selected among those included in each one of predefined polynucleotide sequence sets defined in table 9B.

The detection of an overexpression of genes identified with sets of polynucleotide sequences defined in table 9A, together with detection of an underexpression of genes identified with sets of polynucleotide sequences defined in table 9B allows to classify patients having good or poor prognosis primary breast tumors.

In a preferred embodiment, the tumor cell presenting underexpressed or overexpressed sequences from the polynucleotide library of the invention are breast tumor cells.

In a particular embodiment the polynucleotides of the polynucleotide library of the present invention are immobilized on a solid support in order to form a polynucleotide array, and said solid support is selected from the group consisting of a nylon membrane, nitrocellulose membrane, glass slide, glass beads, membranes on glass support or a silicon chip.

Another object of the present invention concerns a polynucleotide array useful for prognosis or diagnosis of a tumor bearing at least one immobilized polynucleotide library set as previously defined.

The invention also concerns a polynucleotide array useful to differentiate a normal cell from a cancer cell bearing any combination of at least one polynucleotide sequence selected among those included in each one of predefined polynucleotide sequence sets indicated in table 5, useful in differentiating a normal cell from a cancer cell.

Preferably the polynucleotide array useful to differentiate a normal cell from a cancer cell bears any combination of at least one polynucleotide sequence selected among those included in each one of predefined polynucleotide sequence sets indicated in table 5A, and of at least one polynucleotide sequence selected among those included in each one of predefined polynucleotide sequence sets indicated in table 5B.

The invention relates also to a polynucleotide array useful to detect a hormone-sensitive tumor cell bearing any combination of at least one polynucleotide sequence selected among those included in each one of predefined polynucleotide sequence sets defined in table 6.

Preferably the polynucleotide array useful to detect a hormone-sensitive tumor cell bears any combination of at least one polynucleotide sequence selected among those included in each one of predefined polynucleotide sequence sets defined in table 6A together with at least one polynucleotide sequence selected among those included in each one of predefined polynucleotide sequence sets defined in table 6B.

The invention concerns also a polynucleotide array useful to differentiate a tumor in which a lymph node has been invaded by a tumor cell from a tumor in which a lymph node has not been invaded by a tumor cell bearing any combination of at least one polynucleotide sequence selected among those included in each one of predefined polynucleotide sequence sets defined in table 7.

Preferably, the polynucleotide array useful to differentiate a tumor in which a lymph node has been invaded by a tumor cell from a tumor in which a lymph node has been invaded by a tumor cell bears any combination of at least one polynucleotide sequence selected among those included in each one of predefined polynucleotide sequence sets defined in table 7A together with at least one polynucleotide sequence selected among those included in each one of predefined polynucleotide sequence sets defined in table 7B.

The invention also concerns a polynucleotide array useful to differentiate anthracycline-sensitive tumors from anthracycline-insensitive tumors bearing any combination of at least one polynucleotide sequence selected among those included in each one of predefined polynucleotide sequence sets defined in table 8.

Preferably, the polynucleotide array useful to differentiate anthracycline-sensitive tumors from anthracycline-insensitive tumors bears any combination of at least one polynucleotide sequence selected among those included in each one of predefined polynucleotide sequence sets defined in table 8A together with at least one polynucleotide sequence selected among those included in each one of predefined polynucleotide sequence sets defined in table 8B.

The invention concerns also a polynucleotide array useful to classify good and poor prognosis primary breast tumors bearing any combination of at least one polynucleotide sequence selected among those included in each one of predefined polynucleotide sequence set defined in table 9.

Preferably, the polynucleotide array useful to classify good and poor prognosis primary breast tumors bears any combination of at least one polynucleotide sequence selected among those included in each one of predefined polynucleotide sequence sets defined in table 9A together with at least one polynucleotide sequence selected among those included in each one of predefined polynucleotide sequence sets defined in table 9B.

The present invention also concerns a method for detecting differentially expressed polynucleotide sequences that are correlated with a cancer, said method comprising:

obtaining a polynucleotide sample from a patient;

reacting the polynucleotide sample obtained in step (a) with a probe immobilized on a solid support wherein said probe comprises any of the polynucleotide sequences of the libraries previously defined or an expression product encoded by any of the polynucleotide sequences of the libraries previously defined; and

detecting the reaction product of step (b).

Preferably, the polynucleotide sample obtained at step (a) is labeled before its reaction at step (b) with the probe immobilized on a solid support.

The label of the polynucleotide sample is selected from the group consisting of radioactive, colorimetric, enzymatic, molecular amplification, bioluminescent or fluorescent labels.

In a particular embodiment the reaction product of step (c) is quantified by further comparison of said reaction product to a control sample.

In a first embodiment, the polynucleotide sample isolated from the patient and obtained at step (a) is either RNA or mRNA.

In another embodiment the polynucleotide sample isolated from the patient is cDNA is obtained by reverse transcription of the mRNA.

Preferably the reaction step (b) of the method for detecting differentially expressed polynucleotide sequences comprises a hybridization of the sample RNA issued from patient with the probe.

Preferably the sample RNA is labeled before hybridization with the probe and the label is selected from the group consisting of radioactive, calorimetric, enzymatic, molecular amplification, bioluminescent or fluorescent labels.

This method for detecting differentially expressed polynucleotide sequences is particularly useful for detecting, diagnosing, staging, monitoring, predicting, preventing or treating conditions associated with cancer, and particularly breast cancer.

The method for detecting differentially expressed polynucleotide sequences is also particularly useful when the product encoded by any of the polynucleotide sequence or subsequence set is involved in a receptor-ligand reaction on which detection is based.

The present invention is also related to a method for screening an anti-tumor agent comprising the above-depicted method for detecting differentially expressed polynucleotide sequences wherein the sample has been treated with the anti-tumor agent to be screened.

In a particular embodiment the method for screening an anti-tumor agent comprises detecting polynucleotide sequences reacting with at least one library of polynucleotides or polynucleotide sequence set as previously defined or of products encoded by said library in a sample obtained from a patient.

Tumor Samples and RNA Extraction

To avoid any bias of selection as to the type and size of the tumors, the RNAs to be tested were prepared from unselected samples. Samples of primary invasive breast carcinomas were collected from 34 patients undergoing surgery at the Institute Paoli-Calmette. After surgical resection, the tumors were macrodissected: a section was taken for the pathologist′ s diagnosis and an adjacent piece was quickly frozen in liquid nitrogen for molecular analyses. The median age of patients at the time of diagnosis was 55 years (range 39, 83) and most of them were post-menopausal. Tumors were classified according to the WHO histological typing of breast tumors in: 29 ductal carcinomas, 2 lobular carcinomas, 1 mixed ductal and lobular carcinoma, and 2 medullar carcinomas. They had various sizes, inferior or equal to 20 mm (n 13), between 20 and 50 mm (n=18) or superior to 50 mm (n=3), axillary′ s lymph node status (negative: 19 tumors, positive: 15 tumors), SBR grading (I: 3 tumors, II: 20 tumors, III: 10 tumors, not evaluable: 1 tumor), and estrogen receptor status (ER) evaluated by immunohistochemical assay (23 ER-positive, 11 ER-negative). ER positivity cutoff value was 10%. Adjuvant treatment with radiotherapy and when necessary multi-agent anthracycline-based chemotherapy (n=16) was given to patients according to local practice.

Total RNA was extracted from tumor samples by standard methods (43). Total RNA from normal breast tissue was obtained from Clontech (Palo Alto, Calif.): RNA was isolated from 8 tissue specimens from Caucasian females, age range 23-47. RNA integrity was controlled by denaturing formaldehyde agarose gel electrophoresis and Northern blots using a 28S-specific oligonucleotide.

cDNA Arrays Preparation

Gene expression was analyzed by hybridization of arrays with radioactive probes. The arrays contained PCR products of 5 control clones, and 180 IMAGE human cDNA clones selected with practical criteria (3′ sequence of mRNA, same cloning vector, host bacteria and insert size). This represented 176 genes (4 genes were represented by 2 different clones): 121 with proven or putative implication in cancer and 55 implicated in immune reactions (the list is available on the website: http:/tagc.univ-mrs.fr/pub/Cancer/). Their identity was verified by 5′ tag-sequencing of plasmid DNA and comparison with sequences in the EST (dbEST) and nucleotide (GenBank) databases at the NCBI. Identity was confirmed for all but 14 clones without significant gene similarity, which were referenced by their GenBank accession number. The control clones were: Arabidopsis thaliana cytochrome c 554 gene (used for hybridization signal normalization), 3 poly(A) sequences of different sizes and the vector pT 7T 3D (negative controls).

PCR amplification, purification and robotical spotting of PCR products onto Hybond-N+ membranes (Amersham) were done according to described protocols (4). All PCR products were spotted in duplicate. For normalization purpose, the c 554 gene was spotted 96-fold scattered over the whole membrane.

cDNA Array Hybridizations

Hybridizations were done successively with a vector oligonucleotide (to precisely determine the amount of target DNA accessible to hybridization in each spot), then after stripping of vector probe, with complex probes made from the RNAs (4). Each complex probe was hybridized to a distinct filter. Probes were prepared from total RNA with an excess of oligo(dT 25) to saturate the poly(A) tails of the messengers, and to insure that the reverse transcribed product did not contain long poly(T) sequences. A precise amount of c 554 mRNA was added to the total RNA before labeling to allow normalization of the data.

Five ng of total RNA (˜100 ng of mRNA) from tissue samples were used for each labeling. Probe preparation and hybridization of the membranes were done according to known procedures (http:/tagc.univ-mrs.fr/pub/Cancer/). Hybridization was done in excess of target (15 ng of DNA in each spot) and binding of cDNAs to the targets was linear and proportional to the quantity of cDNA in the probe.

Detection and Quantification of cDNA Array Hybridization Signals

Quantitative data were obtained using an imaging plate device. Hybridization signal detection with a FUJI BAS 1500 machine and quantification with the HDG Analyzer software (Genomic Solutions, Ann Arbor, Mich.) were done as previously described (http:/tagc.univ-mrs.fr/pub/Cancer/). Quantification was done by integrating all spot pixel intensities and substracting a spot background value determined in the neighboring area. Spots were located with a LaPlacian transformation. Spot background level was the median intensity of all the pixels present in a small window centered on the spot and which were not part of any spot (44). Quantified data were normalized in three steps and expressed as absolute gene expression levels (i.e. in percentage of abundance of individual mRNA with respect to mRNA within the sample), as described (4).

Array Data Analysis

Before analysis of the results, the reproducibility of the experiments was verified by comparing duplicate spots, or one hybridization with the same probe on two independent arrays, or two independent hybridizations with probes prepared from the same RNA. In every case, the results showed good reproducibility with respective correlation coefficients of 0.95, 0.98 and 0.98 (data not shown). Moreover, genes represented by two different clones on the array, such as CDK 4 (SEQ ID No: 288) or ETV 5 (SEQ ID No: 300), displayed similar expression profiles for the two clones in all samples. This reproducibility was sufficient to consider a 2-fold expression difference as significantly differential.

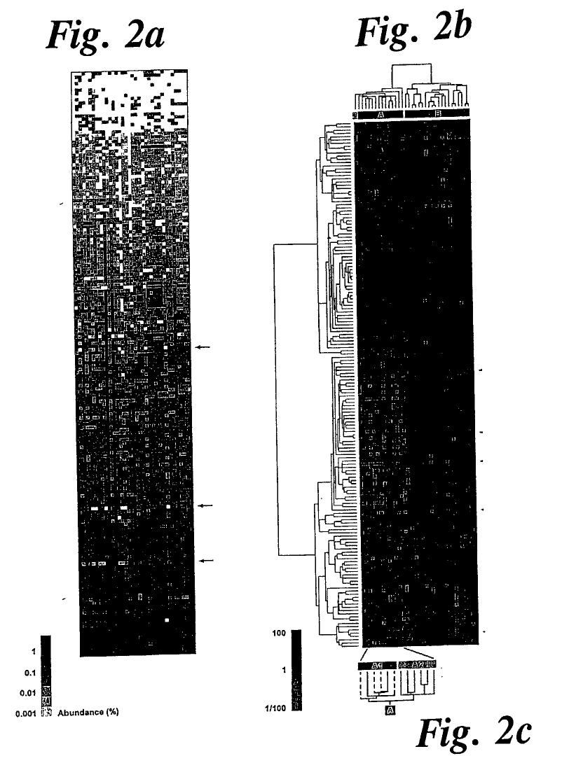

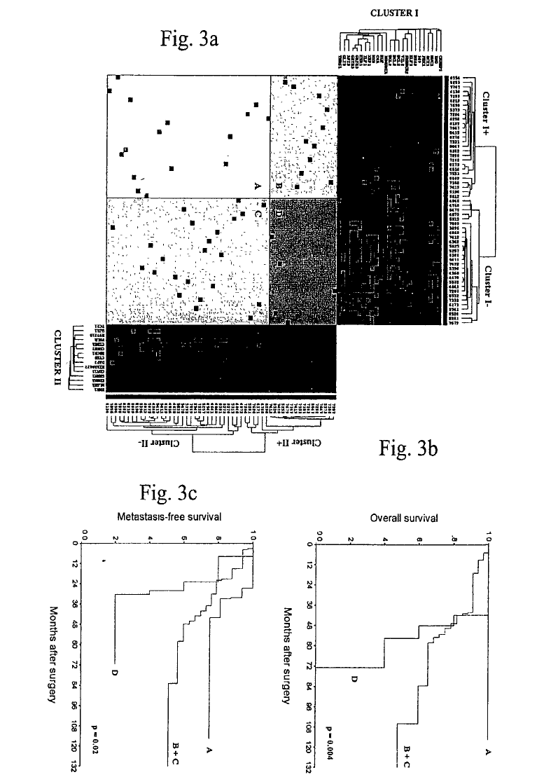

For graphical representation, data were displayed as absolute expression levels (FIG. 2a). For better visualization of clustering, results were log-transformed and displayed as relative values median-centered in each row and in each column (FIG. 2b). Hierarchical clustering was applied to the tissue samples and the genes using the Cluster program developed by Eisen (45) (average linkage clustering using Pearson correlation as similarity metric). Results in FIGS. 2 and 3 were displayed with the TreeView program (45).

Subsequent analysis was done using Excel software (Microsoft) and statistical analyses with the SPSS software. Metastasis-free survival and overall survival were measured from diagnosis until the first metastatic relapse or death respectively. They were estimated with the Kaplan-Meier method and compared between groups with the Log-Rank test. Correlations of gene pairs based on expression profiles were measured with the correlation coefficient r. The search for genes with expression levels correlated with tumor parameters was done in several successive steps.

First, genes were detected by comparing their median expression level in the two subgroups of tumors discordant according to the parameter of interest. The median values rather than the mean values were used because of the high variability of the expression levels for many genes, resulting in a standard deviation of expression level similar or superior to the mean value and making comparisons with means impossible. Second, these detected genes were inspected visually on graphics, and finally, an appropriate statistical analysis was applied to those that were convincing to validate the correlation. Comparison of GATA3 (SEQ ID No: 78) expression between ER-positive tumors and ER-negative tumors was validated using a Mann-Witney test. Correlation coefficients were used to compare the gene expression levels to the number of axillary nodes involved.

Northern Blot Analysis

Seventy-nine breast tumors, including 22 of the 34 tested on the arrays, were analyzed for GATA3 (SEQ ID No: 78) expression by Northern blot hybridization. RNA extraction from tumor samples and Northern blots were done as previously described (43). The GATA3 probe was prepared from the IMAGE cDNA clone 129757 (SEQ ID No: 78), which corresponds to the 3′ region (from +843 to +1689) of the GATA3 cDNA sequence (GenBank accession no. X55122). The insert (846 bp) was obtained by digestion of the clone with EcoRI and Pael enzymes. Northern blots were stripped and re-hybridized using an â-actin probe (46).

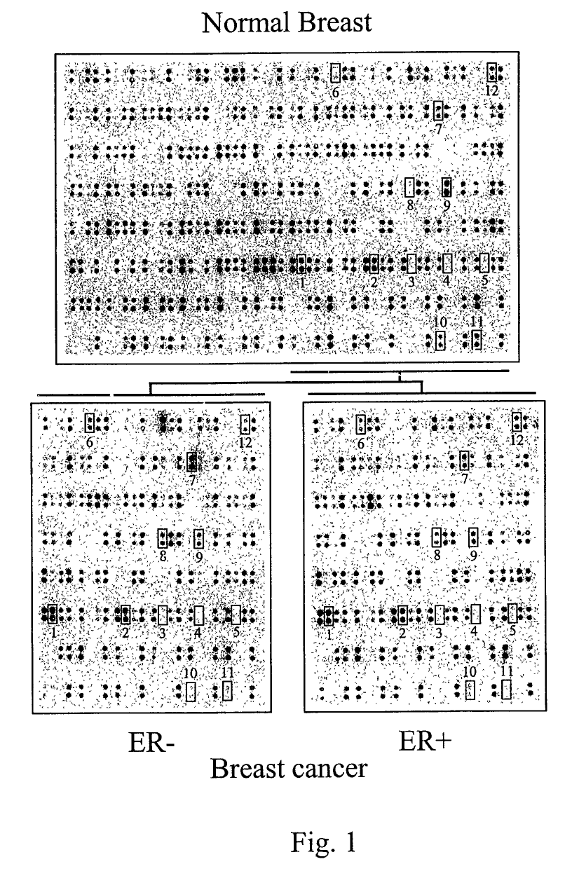

FIG. 1 shows an example of differential gene expression between normal breast tissue (NB) and breast tumor samples. Each cDNA array on Nylon filter was hybridized with a complex probe made from 5 fg of total RNA. The top image corresponds to the whole membrane. For the two bottom images, only the right portion of the membranes is shown. Numbers below the spots indicate housekeeping genes (1, GAPDH and 2, actin), negative control clones (3, 4 and 5) and examples of genes differentially expressed between NB and breast tumor (6, stromelysin 3 (SEQ ID No: 346); 7, ERBB2 (SEQ ID No: 119); 8, MYBL2 (SEQ ID No: 310); 9, FOS (SEQ ID No: 318); 10, TGFáR 3; 11, desmin (SEQ ID No: 170)), and between ER- breast tumor and ER+ breast tumor (12, GATA3).

FIG. 2 is a representation of expression levels of 176 genes in normal breast tissue (NB) and 34 samples of breast carcinoma. Each column corresponds to a single tissue, and each row to a single gene. (a) The results are expressed as percentage abundance of individual mRNA within the sample, and are represented using a blue color scale. The color scale (log scale with a 3-fold interval) indicated at the bottom left ranges from light blue (expression level ≧0.001%) to dark blue (expression level >3%). White squares indicate clones with undetectable expression levels and gray squares indicate missing data. The tissue samples are arbitrarily ordered and the clones are ordered from top to bottom according to increasing median expression levels. Horizontal black arrows on the right of the figure mark three clones with highly variable expression levels between the tumors (stromelysin 3 (SEQ ID No: 346), IGF2 (SEQ ID No: 61), GATA3 (SEQ ID No: 78) from top to bottom). (b) The results are shown as relative expression levels (relative to the median value of each row and each column) and are represented with a color scale indicated at the bottom left ranging from 1/100 to 100 fold changes (gray squares: missing data). Eighteen clones with median expression level equal to zero in the 34 tumors are omitted. The clustering program arranges samples (n=35) along the horizontal axis so that those with the most similar expression profiles are placed adjacent to each other. Similarly, clones (n=162) are near each other along the vertical axis if they show a strong expression profile correlation across all tissues. The length of the branches of the dendrograms capturing respectively the samples (top) and the clones (left) reflects the similarity of the related elements. Two groups of tumors are separated and color coded: group A (blue) and group B (orange). Horizontal black and horizontal red arrows on the right of the figure respectively mark three genes with highly variable expression levels between the tumors (IGF2 (SEQ ID No: 61), GATA3 (SEQ ID No: 78), stromelysin 3 (SEQ ID No: 346) from top to bottom) and four pairs of different clones representing four genes. (c) Zoom representation of group A from FIG. 2b, excluding the two outlyer tumors at the right. The clustering separates two subgroups of tumors, A1 and A2. The dotted branches correspond to tumors associated with metastatic relapse and death. Follow-up was longer in A2 than in A1 (median 81 months for A2 versus 47 months for A1).

FIG. 3 is prognostic classification of breast cancer by gene expression profiling showing that gene expression-based tumor classification correlates with clinical outcome. The 12 samples of group A (see FIG. 2band 2c) were reclustered using the top 32 differentially expressed genes between A1 and A2 subgroups. Data were displayed as in FIG. 2band shown with the same color key. The hierarchical clustering was applied to expression data from the 23 clones, out of 32, of which expression levels presented an at least two-fold change in at least two samples (out of 12). Two subgroups of tumors A1 and A2 are shown as well as two groups of differentially expressed clones. The dotted branches of tumor cluster A1 correspond to samples associated with metastatic relapse and death. FIG. 3a shows two-dimensional representation of hierarchical clustering results shown in FIGS. 2a and 2b. The analysis delineates 4 groups of tumours A, B, C and D. Black squares indicate patients alive at last follow-up visit and red squares indicate patients who died. Three classes of patients with a statistically different clinical outcome were defined according to gene expression profiles: class A (n=16), class B+C (n=34), class D (n=5). FIG. 3b illustrates a Kaplan-Meier plot of overall survival of the 3 classes of patients (p<0.005, log-rank test). And FIG. 3c illustrates a Kaplan-Meier plot of metastasis-free survival of the 3 classes of patients (p<0.05, log-rank test).

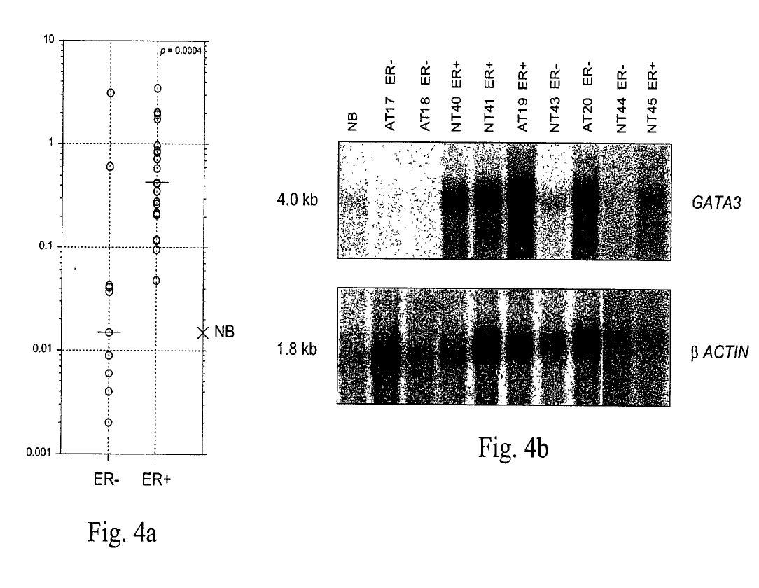

FIG. 4 shows the correlation of GATA3 (SEQ ID No: 78) expression with ER phenotype. (a) The expression levels of GATA3 in 34 breast cancer samples (y axis) monitored by cDNA array analysis are reported in percentage of abundance of individual mRNA with respect to mRNA within the sample (log scale). GATA3 is significantly overexpressed in the ER-positive tumors (n=23) versus the ER-negative tumors (n=11) using the Mann-Witney test (p=0.0004). The expression level of GATA3 in normal breast tissue is reported on the right (NB). (b) Northern blot analysis of GATA3 in normal breast sample (NB) and 9 breast cancer samples (AT: tumor analyzed with cDNA array and Northern blot; NT: tumor analyzed with Northern blot). Blots were probed successively with cDNA from GATA3 (top) and â-actin (bottom). ER status is indicated for each tumor sample.

Data Representation

FIG. 1 shows examples of hybridizations of cDNA arrays with probes made from RNA extracted from normal breast tissue and breast tumors.

The crude results of all hybridizations were processed to be presented either as absolute or relative values in schematic figures. The normalization procedure allowed display of absolute values expressed in percent of abundance of mRNA in the probe as shown in FIG. 2a. Each level of the blue color ladder represents a 3-fold interval of absolute abundance of mRNA. Each column corresponds to a tissue sample and each row to a gene. For graphic purposes, genes were ordered from top to bottom according to increasing median expression levels. Tumor samples were not ordered. The values in each sample displayed a wide range of intensities (3 decades in log scale) corresponding to expression levels ranging from approximately 0.002% to 5% of mRNA abundance. Many genes (see for example stromelysin 3 (SEQ ID No: 346), IGF2 (SEQ ID No: 61) and GATA3 (SEQ ID No: 78), arrows) displayed highly variable expression levels across all tumor samples, scattered over the whole dynamic range of values. A representation of relative values is shown in FIG. 2b. Absolute values were log-transformed, omitting 18 clones whose median intensity was equal to zero across all tissues. Data for each of the 162 remaining clones were then median-centered, as well as data for each sample, so that the relative variation was shown, rather than the absolute intensity. A color scale was used to display data: red for expression level higher than the median and green for expression level lower than the median. The magnitude of the deviation from the median was represented by the color intensity. A hierarchical clustering program was then applied to group the 35 samples according to their overall gene expression profiles, and to group the 162 clones on the basis of similarity of their expression levels in all tissues. This resulted in a picture highlighting groups of correlated tissues and groups of correlated genes as depicted by dendrograms.

Breast Tumor Classification

As shown in FIG. 2b, the clustering algorithm identified two groups of samples, designated A (n=15, including normal breast, NB) and B (n=20). These groups were similar with respect to patient age, menopausal status at diagnosis, SBR grading and tumor pathological size. However, 72% of tumors in group A were node-positive and 75% in group B were node-negative. Moreover, 80% of the tumors in group B were estrogen receptor (ER) positive and 50% in group A were ER-negative. With a median follow-up of 44 months after diagnosis, overall survival was different between A and B groups: 5 women died in A (median follow-up 58 months) and 1 in B (median follow-up 40 months). But the frequency of metastatic relapse was relatively similar in the two groups, with 5 women who relapsed in A and 6 in B. Because the time between the diagnosis of metastasis and last follow-up is too short in B, a longer follow-up is needed to determine if these two different groups, defined with expression profiles, have really a different outcome with respect to overall survival.

In the group A of 15 samples, three samples (normal breast and two tumors) were different from each other and from the other 12 samples. The latter constituted two subgroups of tumors, A1 (n=6) and A2 (n=6), which could be further separated by clustering as shown in FIG. 2c. The 12 tumors had a uniformly high risk of metastatic relapse according to conventional prognostic features as shown in Table 1. Most of them had received comparable adjuvant anthracycline-based chemotherapy after surgery, with more women treated in the A1 subgroup. Interestingly, these two subgroups, which could not be distinguished with commonly used histoclinical features, had a very different clinical outcome: there were 4 metastatic relapses and 4 deaths in A1 (median follow-up: 44 months). In contrast and despite a longer median follow-up (90 months), no metastasis or death occurred in A2. This resulted in a significant better metastasis-free survival (p<0.01) and overall survival (p<0.005) for group A2 than for group A1 tumors. No such subgrouping could be done in B.

TABLE 1

| Subgroup | A1 | | | | | | A2 | | | | | |
|---|---|---|---|---|---|---|---|---|---|---|---|---|
| Tumor position in the cluster | 1 | 2 | 3 | 4 | 5 | 6 | 7 | 8 | 9 | 10 | 11 | 12 |
| Age, years | 46 | 58 | 60 | 63 | 51 | 58 | 46 | 47 | 50 | 47 | 46 | 66 |
| Nodal status | 1 | 0 | 0 | 16 | 13 | 37 | 10 | 4 | 1 | 2 | 0 | 0 |
| Histological size, mm | 60 | 20 | 26 | 35 | 20 | 30 | 27 | 25 | 30 | 25 | 20 | 22 |
| SBR grade | \|\| | \|\|\| | \|\| | \|\|\| | \|\| | \|\|\| | \|\| | \|\| | \|\| | \|\| | \|\| | \|\|\| |
| ER status | neg | neg | neg | neg | neg | neg | pos | neg | pos | pos | pos | pos |
| Adjuvant chemotherapy | yes | yes | no | yes | yes | yes | yes | yes | no | yes | no | no |
| Metastasis | yes | no | yes | yes | no | yes | no | no | no | no | no | no |
| Follow-up, months | 58 | 106 | 35 | 47 | 41 | 31 | 85 | 98 | 95 | 49 | 19 | 141 |
| Patients status | D | A | D | D | A | D | A | A | A | A | A | A |

Patient characteristics in subgroups A1 and A2. The 12 tumors are numbered from 1 to 12 according to their position from left to right in the clustering graphic displayed in FIG. 3. Adjuvant chemotherapy was anthracycline-based. In the line concerning the patient status, A means alive and D means death from cancer progression.

Genes responsible for group A substructure were searched. These are potentially relevant to the prognosis and the sensitivity to chemotherapy in these tumors. Thirty-two genes out of 188 were identified by comparing their median expression level in A1 vs A2. Then, the 12 tumors were reclustered using the expression profiles of these genes as shown in FIG. 3. The same subgroups A1 and A2 were evident and separated by 2 groups of genes: as expected, high expression of ERBB2 (SEQ ID No: 119), MYC (SEQ ID No: 75) and EGFR (SEQ ID No: 137) was associated with bad prognosis subgroup A1 (6-8), and that of E-cadherin (SEQ ID No: 328) and the proto-oncogene MYB (SEQ ID No: 355) with good prognosis subgroup A2 (9, 10). For most of the other genes, these results may stimulate new investigations. Differentiation state is a good prognostic factor in breast cancer and, accordingly, genes associated with cell differentiation, such as GATA3 (SEQ ID No: 78) (11) and CRABP2 (SEQ ID No: 158) (12), had a high level of expression in the better outcome group. The high expression of Ephrin-Al mRNA in the bad prognosis subgroup suggests a role of this growth factor in breast cancer and can be paralleled with its up-regulation during melanoma progression (13).

Differential Gene Expression Between Normal Breast and Breast Tumors

To identify genes differentially expressed between breast tumors (T) and normal breast (NB), the NB value for each gene was compared to its expression level in each tumor. When the expression level of a gene in NB was undetectable, only qualitative information could be deduced and the mRNA was considered as differentially expressed if the signal intensity in the tumor was superior to the reproducibility threshold (0.002% of mRNA abundance). In the other cases, differential expression was defined by an at least 2-fold expression difference. Also, the number of tumors where it was over- or underexpressed was measured. Table 2 shows a list of the top 20 over- and underexpressed genes. For these genes, the T/NB ratio is reported, where T represented their median expression value in the 34 tumors. This ratio ranged from 2.70 (ABCC 5; (SEQ ID No: 325) to 17.76 (GATA3; (SEQ ID No: 78) for the overexpressed genes, and from 0.00 (desmin, (SEQ ID No: 170) to 0.29 (APC; (SEQ ID No: 56) for the underexpressed genes.

TABLE 2

| | | | | |
|---|---|---|---|---|
| Clone ID | Gene/Protein identity | Gene symbol | Chrom. location | N T/NB |

Overexpressed genes

| | | | | |
|---|---|---|---|---|
| 154343 | Granzyme H | GZMH | 14q11.2 | 32 9.51 |
| 235947 | Stromelysin 3 | STMY3 | 22q11.2 | 31 15.92 |
| 207378 | MYB Related Protein B | MYBL2 | 20q13.1 | 31 (a) |
| 153275 | Cellular Retinoic Acid Binding Protein 2 | CRABP2 | 1q21.3 | 29 7.16 |
| 129757 | GATA-binding protein 3 | GATA3 | 10p15 | 28 17.76 |
| 120649 | T-Lymphocyte surface CD2 antigen | CD2 | 1p13.1 | 28 7.54 |
| 109677 | CREB Binding Protein | CREBBP | 16p13.3 | 28 5.08 |
| 172152 | EGFR-binding protein GRB2 | GRB2 | 17q24-q25 | 28 5.00 |
| 66969 | Transcription factor RELB | RELB | 19 | 28 3.61 |
| 182007 | ETS-Related Transcription Factor ELF1 | ELF1 | 13q13 | 27 3.58 |
| 153446 | LIM domain protein RIL | RIL | 5q31.1 | 26 4.03 |
| 203394 | ETS Variant gene 5 (ETS-related molecule) | ETV5 | 3q28 | 25 3.67 |
| 160963 | Thrombospondin 1 | THBS1 | 15q15 | 25 3.39 |
| 188393 | POU domain, class 2, transcription Factor 2 | POU2F2 | 19 | 24 4.02 |
| 187822 | Integrin, beta 2 | ITGB2 | 21q22.3 | 24 3.01 |
| 243907 | Nuclear Factor of Activating T cell Subunit p45 | NF45 | 1 | 24 2.84 |
| 158347 | EST H27202 | EST | | 23 2.91 |
| 230933 | EST AW184517 | EST | | 22 2.85 |
| 212366 | ATP-Binding Cassette, sub-family C (CFTR/MRP), 5 | ABCC5 | 3q27 | 22 2.70 |
| 149401 | Cathepsin D | CTSD | 11p15.5 | 21 2.97 |

Underexpressed genes

| 153854 | Desmin | DES | 2q35 | 34 0.00 |
|---|---|---|---|---|
| 208717 | P55-C-FOS proto-oncogene protein | FOS | 14q24.3 | 33 0.05 |
| 159093 | Transcription Factor AP4 | TFAP4 | 16p13 | 33 0.11 |
| 124340 | Tenascin XA | TNXA | 6p21.3 | 33 0.14 |
| 133738 | Prolactin | PRL | 6p22.2-p21.3 | 32 0.00 |
| 133891 | Chorionic Somatomammotropin Hormone 1 | CSH1 | 17q22-q24 | 32 0.00 |
| 151501 | Tyrosine Kinase Receptor TEK | TEK | 9p21 | 32 0.00 |
| 183030 | Activating Transcription Factor 3 | ATF3 | 1 | 32 0.07 |
| 120916 | Phosphodiesterase I | PDNP2 | 8q24.1 | 32 0.14 |
| 155716 | EST R72075 | EST | | 31 0.00 |
| 208118 | Transforming Growth Factor Beta Receptor Type III | TGFBR3 | 1p33-p32 | 31 0.14 |
| 187547 | Diphtheria Toxin Receptor | DTR | 5q23 | 31 0.17 |
| 108490 | HIV-1 Rev Binding protein | HRB | 2q36 | 31 0.20 |
| 147002 | B-cell CLL/lymphoma 2 | BCL2 | 18q21.3 | 31 0.26 |
| 182610 | Microsomal Glutathione S Transferase 1 | MGST1 | 12p12.3-p12.1 | 31 0.28 |
| 152802 | Phospholipase A2 Membrane Associated, group IIA | PLA2G2A | 1p35 | 30 0.03 |
| 183087 | Interleukin 3 Receptor Alpha chain | IL3RA | Xp22.3; Yp13.3 | 30 0.24 |
| 108571 | Retinoblastoma-Like 2 (p130) | RBL2 | 16q12.2 | 29 0.28 |
| 125294 | Adenomatous Polyposis Coli Protein | APC | 5q21-q22 | 29 0.29 |
| 151767 | FASL Receptor | TNFRSF6 | 10q24.1 | 28 0.27 |
| List of the genes that show the most frequent differential expression between normal breast tissue and 34 breast carcinomas as measured by cDNA array analysis. N indicates the number of tumor samples where the gene is dysregulated (fold change □ 2) compared to normal breast tissue. T/NB represents the ratio: median expression level in 34 breast tumors/expression level in normal breast. (a) MYBL2 transcript displayed a median expression level of 0.025% in breast tumors | | | | |
| # and was undetectable in NB. | | | | |

High expression of mucin I (SEQ ID No: 58), NM 23, ERBB2 (SEQ ID No: 119), FGFRJ (SEQ ID No: 182) and FGFR 2 (SEQ ID No: 15), MYC (SEQ ID No: 75), stromelysin 3 (SEQ ID No: 346), cathepsin D (SEQ ID No: 128) and downregulation of FOS (SEQ ID No: 318), APC (SEQ ID No: 56), RBL2, FAS, BCL2 (SEQ ID No: 117) were found, reflecting what is known about their biology in cancer. GATA3 (SEQ ID No: 78), which codes for a member of the GATA family of zinc finger transcription factors, and CRABP2 (SEQ ID No: 158), encoding one of the two cellular retinoic acid-binding proteins, showed high expression of mRNA, extending previous results on cDNA arrays (4). Differential gene expression among various breast tumors and correlation with histoclinical prognostic parameters

To search for potential prognostic markers in breast cancer, genes with expression levels correlated with conventional histoclinical prognostic parameters were looked for: age of patients, axillary node status, tumor size, histological grade and ER status. No significant correlation was found with age, tumor size and histological grade. However, the expression profiles of some genes correlated with ER status and axillary node involvement.

To identify genes potentially relevant to the hormone-responsive phenotype, the gene expression profiles in ER-positive breast cancers (n=23) versus ER-negative breast cancers (n=11) were compared. Sixteen clones displayed a median intensity of 0 in both groups. Twenty-five presented a fold change superior to 2. Table 3a displays the top 10 over- and underexpressed genes. Among them, the most differentially expressed was GATA3 (SEQ ID No: 78) with a median intensity ratio ER+/ER− of 28.6 and a value for the first quartile of ER-positive tumors superior (5-fold) to the value of the third quartile of the ER-negative tumors as shown in FIG. 4a. The high expression of GATA3 in ER-positive tumors was statistically significant using a Mann-Witney test (p<0.001). All ER-positive tumors and only 18% of ER-negative tumors displayed a GATA3 expression level greatly superior (fold change >3) to the normal breast value. Furthermore GATA3 expression was analyzed by Northern blot hybridization (FIG. 4b) in a panel of 79 breast cancers (21 ER-negative tumors and 58 ER-positive tumors), including 22 of the tumors analyzed with cDNA arrays. It confirmed the array results for those 22 tumors as well as the strong correlation between ER status and GATA3 RNA expression (Mann-Witney test, p<0.0001).

TABLE 3a

| Clone | | | ER+/ |
|---|---|---|---|
| ID | Gene/Protein identity | Gene symbol | ER− |
| 129757 | GATA-binding protein 3 | GATA3 | 28.6 |
| 356763 | Granzyme A | GZMA | 5.7 |
| 248613 | MYB proto-oncogene | MYB | 3.4 |
| 211999 | KIAA1075 protein | KIAA1075 | 3.3 |
| 235947 | Stromelysin 3 | STMY3 | 3.1 |
| 229839 | Macrophage Stimulating 1 | MST1 | 2.8 |
| 153275 | Cellular Retinoic Acid Binding Protein 2 | CRABP2 | 2.7 |
| 301950 | X-box Binding Protein 1 | XBP1 | 2.7 |
| 205314 | Tumor Protein p53 | TP53 | 2.5 |
| 126233 | Insulin-like Growth Factor 2 | IGF2 | 2.4 |
| 66322 | CD3G antigen, Gamma | CD3G | 0.0 |
| 195022 | Interleukin 2 Receptor Gamma chain | IL2RG | 0.0 |
| 111461 | SOX4 Protein | SOX4 | 0.4 |
| 151475 | Epidermal Growth Factor Receptor | EGFR | 0.5 |
| 195022 | Interleukin 2 Receptor Beta chain | IL2RB | 0.5 |
| 130788 | Topoisomerase (DNA) II beta (180 kD) | TOP2B | 0.6 |
| 323948 | SOX9 Protein | SOX9 | 0.6 |
| 183641 | S100 calcium-binding protein Beta | S100B | 0.6 |
| 246620 | EST N53133 | EST | 0.6 |
| 231424 | Glutathione S Transferase Pi | GSTP1 | 0.6 |

To search for genes whose expression profile was correlated with axillary lymph node status, a strong prognostic factor in breast cancer, the group of node-negative tumors (n=19) was compared with the group of tumors with massive axillary extension (10 or more positive nodes). Furthermore, because survival decreases with the increase of the number of tumor-involved lymph nodes and because the expression measurements were quantitative, correlation between the expression levels of these genes and the number of tumor-involved nodes (quantitative variables) was determined. Table 3bshows a list of the top 10 over- and underexpressed genes between these 2 groups. Most of these genes have not been previously reported as associated with node status, but some of these results are in agreement with literature data. The gene encoding the tyrosine kinase receptor ERBB2 (SEQ ID No: 119) was the most significantly overexpressed gene in node-positive tumors and displayed the highest correlation coefficient (r=0.68; p<0.0001).

TABLE 3b

| Clone | | | N−/ |
|---|---|---|---|
| ID | Gene/Protein identity | Gene symbol | 10N+ |
| 129757 | GATA-binding protein 3 | GATA3 | 11.0 |
| 160963 | Thrombospondin 1 | THBS1 | 6.6 |
| 151475 | Epidermal Growth Factor Receptor | EGFR | 5.4 |
| 120916 | Phosphodiesterase I | PDNP2 | 4.9 |
| 183030 | Activating Transcription Factor 3 | ATF3 | 4.6 |
| 211999 | KIAA1075 protein | KIAA1075 | 4.5 |
| 110480 | Nuclear Factor 1 A-type | NF1A | 4.5 |
| 182264 | P-Selectin | SELP | 4.4 |
| 356763 | Granzyme A | GZMA | 4.3 |
| 214008 | E-cadherin | CDH1 | 4.0 |
| 147016 | ERBB2 Receptor Protein-Tyrosine Kinase | ERBB2 | 0.2 |
| 179197 | Protein Phosphatase PP2A, 55 kD Subunit | PP2A BR | 0.2 |
| | | gamma | |
| 231424 | Glutathione S Transferase Pi | GSTP1 | 0.4 |
| 111461 | SOX4 Protein | SOX4 | 0.4 |
| 195022 | Interleukin 2 Receptor Beta chain | IL2RB | 0.4 |
| 220451 | Zinc Finger protein 144 | ZNF144 | 0.5 |
| 125413 | Mucin 1 | MUC1 | 0.6 |
| 290007 | CD44 antigen, epithelial form | CD44 | 0.6 |
| 108571 | Retinoblastoma-Like 2 (p130) | RBL2 | 0.7 |
| 130788 | Topoisomerase (DNA) II Beta (180 kD) | TOP2B | 0.7 |

List of genes differentially expressed between ER-positive and ER-negative breast tumors (a) and between axillary lymph node-negative tumors and tumors with 10 or more involved lymph nodes (b).

Gene clustering from FIG. 2b showed groups of genes with correlated expression across samples. When different clones represented the same gene, they were clustered next to each other (red arrows). Correlation coefficients between gene pairs in the 34 tumors were often high (1% of the 13,041 gene pairs showed a correlation coefficient superior to 0.95—not shown). An example of highly correlated gene expression is that of BCL2 (SEQ ID No: 117) and RBL2. Such correlated expression, although it has not been described in the literature, probably reflects a common mechanism of regulation for these two genes. Furthermore, these genes also exhibited significant correlated expression with other genes such as PPP2CA (SEQ ID No;184), AKT2 (SEQ ID No: 254), PRKCSH (SEQ ID No: 264) or TNFRSF6/FAS SEQ ID No.143). In particular, a striking correlated expression between BCL2 and FAS could be observed (r=0.91; data not shown). The exact meaning of this correlation is unknown, although it may reflect the necessary balance between apoptosis and anti-apoptosis for cell survival.

Although in human cancer the proportion of changes that is reflected at the RNA level is not known, monitoring gene expression patterns appears as a very promising way of increasing the knowledge of the disease. Several different types of cancer have been investigated using cDNA arrays: cervical (14), hepatocellular (15), ovarian (16), colon (17) and renal carcinomas (18), glioblastomas (19), melanomas (20) (21), rhabdomyosarcomas (22), acute leukemias (23) and lymphomas (24). In breast cancer, pioneering studies have yielded the first expression patterns (4, 25-31). They have in particular addressed the important issue of molecular differences in hormone-responsive and non-responsive breast tumors. Thus, Yang et al. (28) and Hoch et al. (25) compared expression profiles of breast carcinoma cell lines known to represent these two categories and identified a few genes with differential expression. One of these genes was GATA3. In these studies, cell lines were mostly used and tumor samples were rarely tested and generally in small numbers. The first study analyzing the expression profiles of a large series of breast cancers was published recently (32), but no correlation with clinical outcome was mentioned.

Several interesting points can be made based on the present experimentation. First, the differences in expression patterns among the tumors provided molecular transcriptional evidence of the histoclinical heterogeneity of breast cancer. This diversity was multifactorial, linked to many different genes, highlighting the interest of high throughput analysis in this context. It was possible, with a hierarchical clustering program integrating the expression profiles, to separate normal breast tissue from most tumors and, moreover, to identify two different groups of tumors. Most importantly, two different subgroups of tumors with a very distinct clinical outcome that could not be predicted with classical prognostic factors have been identified by clustering. Indeed, all these tumors had a theoretically bad prognosis as evaluated by current histoclinical tools. All these patients would be at the present time treated with adjuvant chemotherapy, but without the capacity for the physicians to identify patients who will benefit from this treatment and those who will not benefit.

Gene expression profiles were able to make this discrimination. Such predictive tools have important therapeutic implications. Patients with features of poor prognosis are candidates for other treatment than standard chemotherapy, avoiding loss of time and toxicities related to first-line chemotherapy. These results suggest that the histoclinical category of poor prognosis breast cancer, currently treated with adjuvant anthracycline-based chemotherapy, groups together at least two molecularly distinct subgroups of tumors with different outcome which would require distinct chemotherapy regimens. Expression profiles could thus provide a new and more accurate way of classifying breast tumors of poor prognosis and managing patients.

Similarly, despite molecular heterogeneity, significant correlations between the expression level of genes (GATA3 (SEQ ID No: 78), ERBB2 (SEQ ID No: 119)) and histological tumor parameters were identified. The ER-positivity in breast cancer has been correlated with tumor differentiation, low proliferating rate, favorable prognosis and response to hormonal therapy. The relation between hormone sensitivity of breast cancer and ER status is not perfect, and it is possible that some genes related to ER expression are more important than ER to characterize the hormone-sensitive phenotype. These genes could serve as predictive factors to guide the therapy.

GATA3 mRNA expression was highly correlated with ER status. GATA3, which is not estrogen-regulated (25), is a transcription factor that could regulate the expression of genes involved in the ER-positive phenotype. Among the other genes that were found associated with ER status during the experimental work leading to the present invention, some, such as MYB (SEQ ID No: 355) (10), stromelysin 3 (SEQ ID No: 346) (33), and CRABP2 (SEQ ID No: 158) (34), have been previously reported expressed at high levels in ER-positive breast tumors. The higher levels of TP53 MnRNA in ER-positive tumors studied were surprising, although in agreement with a recent study (27). Most studies concerning TP53 expression analyzed the protein level rather than the mRNA level, and TP53 protein levels are classically negatively correlated with the ER status (35). The high expression of CRABP2 could be related to the better differentiated status of the ER-positive tumors. The low expression of the three immunity-related genes IL2RB (SEQ ID No: 99), IL2RG (SEQ ID No: 281) and CD3G (SEQ ID No: 416) may be related to the low lymphoid infiltration in these well differentiated tumors. ERBB2 high expression in breast cancer has been associated with a poor prognosis and some resistance to hormonal therapy and chemotherapy (36). It is involved in the regulation of cellular differentiation, adhesion, and motility. The motility-enhancing activity of ERBB2 (37) could be responsible for the increased metastatic potential and the unfavorable prognosis of the breast tumors that overexpress ERBB2. The low expression of E-cadherin (SEQ ID No: 328) and thrombospondin 1 (SEQ ID No: 217) in node-positive tumors are consistent with their putative role in different steps of metastatic spread: E-cadherin is an epithelial cell adhesion molecule whose disturbance is a prerequisite for the release of invasive cells in carcinomas (38) and thrombospondin 1 inhibits angiogenesis (39). Similarly, the high expression of the molecule surface antigen Mucin 1 in node-positive tumors (40) can reduce cell-cell interactions facilitating cell detachment and metastasis. CD44 (SEQ ID No: 376), encoding a transmembrane glycoprotein involved in cell adhesion and lymph node homing (41) was expressed at high levels in node-positive tumors as well as GSTPI (SEQ ID No: 336) (Glutathione-S-Transferase Pi), recently reported associated with increased tumor size (27).

Second, there were a number of genes with highly correlated expression patterns. Gene correlations have already been reported with larger series of genes, essentially under dynamic experimental conditions (42) and recently in steady states (17). Here, correlations were based on expression profiles of a relatively small but selected series of genes and in steady states represented by different breast tumors. Gene correlations are potentially useful tools for cancer research in two ways: i) they can provide information about the general regulation circuitry of a cancerous cell, allowing the identification of regulatory elements controlling expression networks; ii) they offer the possibility of reducing the complexity of the system analyzed by replacing, for example, the intensities of a large number of genes present in a gene cluster by their respective mean intensities.

Finally, these results highlight the great potential of cDNA array in cancer research. The gene expression profiles confirmed the heterogeneity of breast cancer, and most importantly allowed us to identify, among a series of poor prognosis breast tumors, two subtypes of the disease not yet recognized with usual histoclinical parameters but with a different clinical outcome after adjuvant chemotherapy. Furthermore, the present invention allows detection of genes of which expression was correlated with classical prognostic factors.

Table 4 displays a library of polynucleotides SEQ ID NO: 1 to SEQ ID NO: 468 corresponding to a population of polynucleotide sequences underexpressed or overexpressed in cells derived from tumors, more particularly breast tumors, and their respective complements.

TABLE 4
CORRELATION BETWEEN SEQ ID NO AS FILED WITH US PROVISIONAL APPLICATION
N^{o} 60/254,090 and SEQ ID NO FILLED WITH NEW APPLICATION

| Gene | | | | Provisional | Provisional | Current, | Current, | Current, |
|---|---|---|---|---|---|---|---|---|
| Symbols | N^{o} | Name | Image | Seq3′ | Seq5′ | Seq3′ | Seq5′ | (mRNA) |
| GATA3 | 1 | GATA-binding pro- | 129757 | SEQ ID No:1 | | SEQ ID No:76 | SEQ ID No:77 | SEQ ID No:78 |
| | | tein 3 (GATA3) | | | | | | |
| MYB | 2 | v-myb avian myelo- | 248613 | | SEQ ID No:2 | 0 | SEQ ID No:354 | SEQ ID No:355 |
| | | blastosis viral onco- | | | | | | |
| | | gene homolog | | | | | | |
| | | (MYB) | | | | | | |
| KIAA 1075 | 3 | KIAA 1075 protein | 211999 | SEQ ID No:3 | SEQ ID No:4 | SEQ ID No:322 | SEQ ID No:323 | 0 |
| STMY3 | 4 | matrix metallopro- | 235947 | SEQ ID No:5 | | SEQ ID No:345 | 0 | SEQ ID No:346 |
| | | teinase 11 (strom- | | | | | | |
| | | elysin 3) (MMP11) | | | | | | |
| | | (ex STMY3) | | | | | | |
| HGFL | 5 | macrophage-stim- | 229839 | SEQ ID No:6 | SEQ ID No:7 | SEQ ID No:331 | SEQ ID No:332 | SEQ ID No:333 |
| | | ulating protein | | | | | | |
| | | (MST1) (ex HGFL) | | | | | | |
| CRABP | 6 | cellular retinoic | 153275 | SEQ ID No:8 | SEQ ID No:9 | SEQ ID No:156 | SEQ ID No:157 | SEQ ID No:158 |
| | | acid-binding protein | | | | | | |
| | | 2 (CRABP2) | | | | | | |
| XBP1 | 7 | X-box binding pro- | 301950 | SEQ ID No:10 | SEQ ID No:11 | SEQ ID No:385 | SEQ ID No:386 | SEQ ID No:387 |
| | | tein 1 (XBP1) | | | | | | |
| TP53 | 8 | tumor protein p53 | 205314 | | SEQ ID No:12 | SEQ ID No:442 | 0 | 0 |
| | | (Li-Fraumeni syn- | | | | | | |
| | | drome) (TP53) | | | | | | |
| IGF2 | 9 | insulin-like growth | 126233 | SEQ ID No:13 | SEQ ID No:14 | SEQ ID No:59 | SEQ ID No:60 | SEQ ID No:61 |
| | | factor 2 (somato- | | | | | | |
| | | medin A) (IGF2), | | | | | | |
| CD3G | 10 | CD3G antigen, | 66322 | SEQ ID No:15 | SEQ ID No:16 | SEQ ID No:414 | SEQ ID No:415 | SEQ ID No:416 |
| | | gamma polypeptide | | | | | | |
| | | (TiT3 complex) | | | | | | |
| | | (CD3G) | | | | | | |
| IL2RG | 11 | interleukin 2 recep- | 195022 | SEQ ID No:17 | SEQ ID No:18 | SEQ ID No:279 | SEQ ID No:280 | SEQ ID No:281 |
| | | tor, gamma (severe | | | | | | |
| | | comnbined immuno- | | | | | | |
| | | deficiency) (IL2RG) | | | | | | |
| SOX4 | 12 | SRY (sex determin- | 111461 | SEQ ID No:19 | SEQ ID No:20 | SEQ ID No:22 | SEQ ID No:23 | SEQ ID No:24 |
| | | ing region Y)-box 4 | | | | | | |
| | | (SOX4) | | | | | | |
| EGFR | 13 | epidermal growth | 151475 | SEQ ID No:21 | SEQ ID No:22 | SEQ ID No:135 | SEQ ID No:136 | SEQ ID No:137 |
| | | factor receptor | | | | | | |
| | | (avian erythroblastic | | | | | | |
| TOP2B | 14 | topIIb mRNA for | 130788 | | SEQ ID No:23 | 0 | SEQ ID No:82 | SEQ ID No:83 |
| | | topoisomerase IIb. | | | | | | |
| S100B | 15 | S100 calcium-bind- | 183641 | | SEQ ID No:24 | 0 | SEQ ID No:255 | SEQ ID No:256 |
| | | ing protein, beta | | | | | | |
| | | (neural) (S100B) | | | | | | |
| EST N53133 | 16 | EST N53133 | 246620 | SEQ ID No:25 | | SEQ ID No:352 | 0 | SEQ ID No:353 |
| GSTP1 | 17 | glutathione S-trans- | 231424 | SEQ ID No:26 | SEQ ID No:27 | SEQ ID No:334 | SEQ ID No:335 | SEQ ID No:336 |
| | | ferase pi (GSTP1) | | | | | | |
| THBS1 | 18 | thrombospondin 1 | 160963 | SEQ ID No:28 | | SEQ ID No:216 | 0 | SEQ ID No:217 |
| | | (THBS1) | | | | | | |
| PDNP2 | 19 | cctonucleotide | 120916 | SEQ ID No:29 | SEQ ID No:30 | SEQ ID No:39 | SEQ ID No:40 | SEQ ID No:41 |
| | | pyrophosphatase/ | | | | | | |
| | | phosphodiesterase | | | | | | |
| | | 2(autotaxin) | | | | | | |
| | | (ENPP2) (ex | | | | | | |
| | | PDNP2) | | | | | | |
| ATF3 | 20 | activating transcrip- | 183030 | SEQ ID No:31 | SEQ ID No:32 | SEQ ID No:250 | SEQ ID No:251 | SEQ ID No:252 |
| | | tion factor 3 (ATF3) | | | | | | |
| NF1A | 21 | (ex NF1A) | 110480 | SEQ ID No:33 | | SEQ ID No:16 | 0 | 0 |
| SELP | 22 | selectinm P (granule | 182264 | | SEQ ID No:34 | SEQ ID No:438 | SEQ ID No:439 | 0 |
| | | membrane protein | | | | | | |
| | | 140kD, antigen | | | | | | |
| | | CD62) (SELP) | | | | | | |
| CDH1 | 23 | cadherin 1, E- | 214008 | SEQ ID No:35 | SEQ ID No:36 | SEQ ID No:326 | SEQ ID No:327 | SEQ ID No:328 |
| | | cadherin (epi- | | | | | | |
| | | thelial) (CDH1) | | | | | | |
| ERBB2 | 24 | v-erb-b2 avian | 147016 | SEQ ID No:37 | | 0 | SEQ ID No:118 | SEQ ID No:119 |
| | | erythroblastic | | | | | | |
| | | leukemia viral | | | | | | |
| | | oncogene homolog | | | | | | |
| | | 2 (neuro/ | | | | | | |
| | | glioblastoma | | | | | | |
| | | derived oncogene | | | | | | |
| | | homolog) (ERBB2) | | | | | | |
| PP2A BR | 25 | (PP2A BR gamma) | 179197 | SEQ ID No:38 | SEQ ID No:39 | SEQ ID No:238 | SEQ ID No:239 | 0 |
| gamma | | | | | | | | |
| ZNF144 | 26 | zinc finger pro- | 220451 | SEQ ID No:40 | SEQ ID No:41 | 0 | SEQ ID No:329 | SEQ ID No:330 |
| | | tein 144 (Mel-18) | | | | | | |
| | | (ZNF144) | | | | | | |
| MUC1 | 27 | mucin 1, transmem- | 125413 | | SEQ ID No:42 | 0 | SEQ ID No:57 | SEQ ID No:58 |
| | | brane (MUC1) | | | | | | |
| CD44 | 28 | CD44E (epithelial | 290007 | SEQ ID No:43 | SEQ ID No:44 | SEQ ID No:374 | SEQ ID No:375 | SEQ ID No:376 |
| | | form) | | | | | | |
| PLA2G2A | 29 | phospholipase A2, | 152802 | SEQ ID No:45 | SEQ ID No:46 | SEQ ID No:147 | SEQ ID No:148 | SEQ ID No:149 |
| | | group IIA (plate- | | | | | | |
| | | lets, synovial | | | | | | |
| | | fluid) (PLA2G2A), | | | | | | |
| | | nuclear gene | | | | | | |
| | | encoding mito- | | | | | | |
| | | chondrial protein | | | | | | |
| ACVRL1 | 30 | activin A receptor | 153350 | SEQ ID No:47 | SEQ ID No:48 | SEQ ID No:159 | SEQ ID No:160 | SEQ ID No:161 |
| | | type II-like | | | | | | |
| | | 1 (ACVRL1) | | | | | | |
| AXL | 31 | AXL receptor tyro- | 112500 | SEQ ID No:49 | SEQ ID No:50 | SEQ ID No:27 | SEQ ID No:28 | SEQ ID No:29 |
| | | sine kinase (AXL) | | | | | | |
| PKU-ALPHA | 32 | KU-alpha, partial | 109569 | | SEQ ID No:51 | 0 | SEQ ID No:5 | SEQ ID No:6 |
| | | cds (new gene | | | | | | |
| | | symbol Tlk2) | | | | | | |
| ABCC5 | 33 | ATP-binding | 212366 | | SEQ ID No:52 | 0 | SEQ ID No:324 | SEQ ID No:325 |
| | | cassette, sub- | | | | | | |
| | | family C (CFTR/ | | | | | | |
| | | MRP), member | | | | | | |
| | | 5 (ABCC5) | | | | | | |
| EDNRB | 34 | endothelial recep- | 154244 | | SEQ ID No:53 | 0 | SEQ ID No:176 | SEQ ID No:177 |
| | | tor type B | | | | | | |
| | | (EDNRB), trans- | | | | | | |
| | | cript variant1 | | | | | | |
| DTR | 35 | diphtheria toxin | 187547 | | SEQ ID No:54 | 0 | SEQ ID No:265 | SEQ ID No:266 |
| | | receptor (hep- | | | | | | |
| | | arin-binding | | | | | | |
| | | epidermal | | | | | | |
| IGF1R | 36 | insulin-like | 150361 | | SEQ ID No:55 | 0 | SEQ ID No:129 | SEQ ID No:130 |
| | | growth factor 1 | | | | | | |
| | | receptor (IGF1R) | | | | | | |
| KIAA0427 | 37 | KIAA0427 | 127507 | SEQ ID No:56 | SEQ ID No:57 | SEQ ID No:65 | SEQ ID No:66 | SEQ ID No:67 |
| CD69 | 38 | CD69 antigen (p60, | 276727 | | SEQ ID No:58 | 0 | SEQ ID No:370 | SEQ ID No:371 |
| | | early, T-cell | | | | | | |
| | | activation anti- | | | | | | |
| | | gen) | | | | | | |
| FGFR4 | 39 | fibroblast | 116781 | SEQ ID No:59 | SEQ ID No:60 | SEQ ID No:36 | SEQ ID No:37 | SEQ ID No:38 |
| | | growth factor | | | | | | |
| | | receptor 4 | | | | | | |
| | | (FGFR4) | | | | | | |
| EST T85683 | 40 | EST T85683 cathe- | 112622 | | SEQ ID No:61 | 0 | SEQ ID No:30 | SEQ ID No:31 |
| | | spin B (CTSB) | | | | | | |
| EST R00569 | 41 | EST R00569 IL2- | 123871 | | SEQ ID No:62 | 0 | SEQ ID No:44 | SEQ ID No:45 |
| | | inducible T- | | | | | | |
| | | cell kinase (ITK) | | | | | | |
| TGFBR3 | 42 | transforming growth | 208118 | SEQ ID No:63 | SEQ ID No:64 | SEQ ID No:311 | SEQ ID No:312 | SEQ ID No:313 |
| | | factor, beta | | | | | | |
| | | receptor III | | | | | | |
| | | (TGFBR3) | | | | | | |
| INSR | 43 | insulin receptor | 151149 | | SEQ ID No:65 | 0 | SEQ ID NO;131 | SEQ ID No:132 |
| | | (INSR) | | | | | | |
| MARK3 | 44 | MAP/microtubule | 110599 | SEQ ID No:66 | SEQ ID No:67 | #N/A | #N/A | #N/A |
| | | affinity-reg- | | | | | | |
| | | ulating kinase 3 | | | | | | |
| | | (MARK3) | | | | | | |
| TIMP2 | 45 | tissue inhibitor | 131504 | | SEQ ID No:68 | 0 | SEQ ID No:86 | SEQ ID No:87 |
| | | of metallopro- | | | | | | |
| | | teinase 2 (TIMP2) | | | | | | |
| EST R85557 | 46 | EST R85557 throm- | 180219 | SEQ ID No:69 | | SEQ ID No:240 | 0 | SEQ ID No:241 |
| | | bospondin 3 | | | | | | |
| | | (THBD3) | | | | | | |
| GNRH1 | 47 | gonadotropin-releas- | 192688 | | SEQ ID No:70 | 0 | SEQ ID No:277 | SEQ ID No:278 |
| | | ing hormone 1 | | | | | | |
| | | (GNHR1) | | | | | | |
| FGFR2 | 48 | fibroblast growth | 110387 | SEQ ID No:71 | SEQ ID No:72 | SEQ ID No:13 | SEQ ID No:14 | SEQ ID No:15 |
| | | factor receptor | | | | | | |
| | | 2 (FGFR2) | | | | | | |
| NFKB2 | 49 | NFKB2 | 114879 | SEQ ID No:73 | | SEQ ID No:35 | 0 | 0 |
| VIL2 | 50 | villin 2 (ezrin) | 124701 | SEQ ID No:74 | SEQ ID No:75 | SEQ ID No:51 | SEQ ID No:52 | SEQ ID No:53 |
| | | (VIL2) | | | | | | |
| ENG | 51 | endoglin (ENG) | 156979 | SEQ ID No:76 | SEQ ID No:77 | SEQ ID No:196 | SEQ ID No:197 | SEQ ID No:198 |
| EPHA2 | 52 | EphA2 (EPHA2) | 162004 | SEQ ID No:78 | | SEQ ID No:221 | 0 | SEQ ID No:222 |
| CREM | 53 | cAMP responsive | 258584 | SEQ ID No:79 | SEQ ID No:80 | SEQ ID No:358 | SEQ ID No:359 | SEQ ID No:360 |
| | | element modulator | | | | | | |
| | | (CREM) | | | | | | |
| ETV5-a | 54 | ets variant | 270549 | SEQ ID No:81 | SEQ ID No:82 | SEQ ID No:368 | SEQ ID No:369 | SEQ ID No:300 |
| | | gene 5 (ETV5) | | | | | | |
| EST N68536 | 55 | EST N68536 MAX- | 298242 | SEQ ID No:83 | SEQ ID No:84 | 0 | SEQ ID No:380 | SEQ ID No:381 |
| | | interacting pro- | | | | | | |
| | | tein 1 (MX11) | | | | | | |
| EST R81126 | 56 | EST R81126 lym- | 146635 | SEQ ID No:85 | SEQ ID No:86 | SEQ ID No:114 | 0 | 0 |
| | | photoxin beta re- | | | | | | |
| | | ceptor (LTBR) | | | | | | |
| POU2F2 | 57 | (POu2F2) | 188393 | SEQ ID No:87 | SEQ ID No:88 | SEQ ID No:271 | 0 | SEQ ID No:272 |
| FLI1 | 58 | Friend leukemia vir- | 198144 | SEQ ID No:89 | SEQ ID No:90 | SEQ ID No:293 | SEQ ID No:294 | SEQ ID No:295 |
| | | us integration 1 | | | | | | |
| | | (FLI1) | | | | | | |
| TIE | 59 | tyrosine kinase with | 144081 | | SEQ ID No:91 | 0 | SEQ ID No:109 | SEQ ID No:110 |
| | | immunoglobulin and | | | | | | |
| | | epidermal growth | | | | | | |
| | | factor homology | | | | | | |
| | | domains | | | | | | |
| | | (TIE) | | | | | | |
| PRLR | 60 | prolactin receptor | 138788 | SEQ ID No:92 | SEQ ID No:93 | SEQ ID No:94 | SEQ ID No:95 | SEQ ID No:96 |
| | | (PRLR) | | | | | | |
| PPP3CA | 61 | protein phosphatase | 110481 | SEQ ID No:94 | SEQ ID No:95 | SEQ ID No:17 | SEQ ID No:18 | SEQ ID No:19 |
| | | 3 (formerly 2B), | | | | | | |
| | | catalytic subunit, | | | | | | |
| | | gamma isoform | | | | | | |
| | | (calcineurin A | | | | | | |
| | | gamma) (PPP3CC) | | | | | | |
| | | (ex PPP3CA) | | | | | | |
| PTPN2 | 62 | protein tyrosine | 161451 | SEQ ID No:96 | SEQ ID No:97 | SEQ ID No:218 | SEQ ID No:219 | SEQ ID No:220 |
| | | phosphatase, non-re- | | | | | | |
| | | ceptor type 2 | | | | | | |
| | | (PTPN2) | | | | | | |
| PGF | 63 | placental growth | 139326 | | SEQ ID No:98 | 0 | SEQ ID No:102 | SEQ ID No:103 |
| | | factor, vascular | | | | | | |
| | | endothelial growth | | | | | | |
| | | factor-related | | | | | | |
| | | protein (PGF) | | | | | | |
| TNFAIP3 | 64 | tumor necrosis | 309943 | SEQ ID No:99 | | SEQ ID No:388 | SEQ ID No:389 | SEQ ID No:390 |
| | | factor, alpha-in- | | | | | | |
| | | duced protein 3 | | | | | | |
| | | (TNFAIP3) | | | | | | |
| PHB | 65 | PHB (prohibitin) | 236008 | SEQ ID No:100 | | SEQ ID No:347 | SEQ ID No:348 | SEQ ID No:349 |
| RIL | 66 | LIM domain pro- | 153446 | | SEQ ID No:101 | 0 | SEQ ID No:162 | SEQ ID No:163 |
| | | tein (RIL) | | | | | | |
| MYBL2 | 67 | v-myb avian mye- | 207378 | SEQ ID No:102 | SEQ ID No:103 | SEQ ID No:308 | SEQ ID No:309 | SEQ ID No:310 |
| | | loblastosis viral | | | | | | |
| | | oncogene homolog- | | | | | | |
| | | like 2 (MYBL2) | | | | | | |
| RELB | 68 | v-rel avian retic- | 66969 | SEQ ID No:104 | SEQ ID No:105 | SEQ ID No:417 | SEQ ID No:418 | SEQ ID No:419 |
| | | uloendotheliosis | | | | | | |
| | | viral oncogene | | | | | | |
| | | homolog B (nuclear | | | | | | |
| | | factor of kappa light | | | | | | |
| | | polypeptide gene | | | | | | |
| | | enhancer in B-cells | | | | | | |
| | | 3) (RELB) | | | | | | |
| EST R97218 | 69 | Est R97218 | 200394 | SEQ ID No:106 | | SEQ ID No:296 | SEQ ID No:297 | 0 |
| GZMH | 70 | granzyme B (gran- | 154343 | SEQ ID No:107 | | SEQ ID No:178 | 0 | SEQ ID No:179 |
| | | zyme 2, cytotoxic | | | | | | |
| | | T-lymphocyte-ass- | | | | | | |
| | | ociated serine es- | | | | | | |
| | | terase 1) (GZMB) | | | | | | |
| | | (ex GZMH) | | | | | | |
| MYC | 71 | c-myc proto-onco- | 129438 | SEQ ID No:108 | SEQ ID No:109 | SEQ ID No:73 | SEQ ID No:74 | SEQ ID No:75 |
| | | gene | | | | | | |
| CASP1 | 72 | caspase 4, apop- | 131502 | | SEQ ID No:110 | SEQ ID No:84 | 0 | SEQ ID No:85 |
| | | tosis-related cy- | | | | | | |
| | | steine protease | | | | | | |
| | | (CASP4) (ex | | | | | | |
| | | CASP1) | | | | | | |
| SYK | 73 | spleen tyrosine | 128142 | SEQ ID No:111 | SEQ ID No:112 | SEQ ID No:68 | SEQ ID No:69 | SEQ ID No:70 |
| | | kinase (SYK) | | | | | | |
| EST H27202 | 74 | EST H27202 trans- | 158347 | SEQ ID No:113 | SEQ ID No:114 | SEQ ID No:204 | SEQ ID No:205 | 0 |
| | | cription factor | | | | | | |
| | | E1AF gene | | | | | | |
| HRB | 75 | syndecan 1) | 108490 | SEQ ID No:115 | SEQ ID No:116 | SEQ ID No:1 | 0 | SEQ ID No:2 |
| | | (SDC1) (ex HRB) | | | | | | |
| SHC1 | 76 | p66shc (SHC) | 153548 | | SEQ ID No:117 | 0 | SEQ ID No:164 | SEQ ID No:165 |
| CSF1 | 77 | colony stimulating | 124554 | SEQ ID No:118 | SEQ ID No:119 | SEQ ID No:48 | SEQ ID No:49 | SEQ ID No:50 |
| | | factor 1 (CSF1) | | | | | | |
| UBE3A | 78 | ubiquitin protein | 141924 | | SEQ ID No:120 | 0 | SEQ ID No:104 | SEQ ID No:105 |
| | | ligase E3A | | | | | | |
| | | (UBE3A) | | | | | | |
| FKHR | 79 | forkhead box | 151247 | | SEQ ID No:121 | 0 | SEQ ID No:133 | SEQ ID No:134 |
| | | O1A (rhabdomyo- | | | | | | |
| | | sarcoma) | | | | | | |
| | | (FOXO1A) (ex | | | | | | |
| | | FKHR) | | | | | | |
| CSF1R | 80 | colony stimulating | 196282 | SEQ ID No:122 | | SEQ ID No:291 | 0 | SEQ ID No:292 |
| | | factor 1 re- | | | | | | |
| | | ceptor (CSF1R) | | | | | | |
| IFI75 | 81 | interferon-induced | 205612 | SEQ ID No:123 | SEQ ID No:124 | SEQ ID No:305 | SEQ ID No:306 | SEQ ID No:307 |
| | | protein 75 (IFI75) | | | | | | |
| GATA1 | 82 | GATA-binding pro- | 109093 | | SEQ ID No:125 | 0 | SEQ ID No:3 | SEQ ID No:4 |
| | | tein 1 (globin | | | | | | |
| | | transcription | | | | | | |
| | | factor 1) (GATA1) | | | | | | |
| STAT1 | 83 | signal transducer | 110101 | | SEQ ID No:126 | 0 | SEQ ID No:11 | SEQ ID No:12 |
| | | and activator of | | | | | | |
| | | transcription 1 | | | | | | |
| | | (STAT1) | | | | | | |
| CREBBP | 84 | CREB binding pro- | 109677 | SEQ ID No:127 | SEQ ID No:128 | SEQ ID No:7 | SEQ ID No:8 | 0 |
| | | tein (Rubinstein- | | | | | | |
| | | Taybi syndrome) | | | | | | |
| | | (CREBBP) | | | | | | |
| IL7R | 85 | interleukin 7 | 129059 | | SEQ ID No:129 | 0 | SEQ ID No:71 | SEQ ID No:72 |
| | | receptor (IL7R) | | | | | | |
| ANXA7 | 86 | annexin A7 | 160580 | | SEQ ID No:130 | 0 | SEQ ID No:214 | SEQ ID No:215 |
| | (AN- | | | | | | | |
| | XA7) | | | | | | | |
| TNXA | 87 | tenascin XA | 124340 | | SEQ ID No:131 | 0 | SEQ ID No:46 | SEQ ID No:47 |
| | (TN- | | | | | | | |
| | XA) | | | | | | | |
| CNBP1 | 88 | zinc finger pro- | 251963 | SEQ ID No:132 | | SEQ ID No:356 | 0 | SEQ ID No:357 |
| | | tein 9 (a cellular | | | | | | |
| | | retroviral nucleic | | | | | | |
| | | acid binding pro- | | | | | | |
| | | tein) (ZNF9) (ex | | | | | | |
| | | CNBP1) | | | | | | |
| CDK4-a | 89 | cyclin-dependent | 204586 | SEQ ID No:133 | SEQ ID No:134 | SEQ ID No:301 | SEQ ID No:302 | SEQ ID No:288 |
| | | kinase 4 (CDK4) | | | | | | |
| CSNK2B | 90 | gene for casein | 153879 | | SEQ ID No:135 | 0 | SEQ ID No:171 | SEQ ID No:172 |
| | | kinase II subunit | | | | | | |
| | | beta (EC 2.7.1.37). | | | | | | |
| EFNA1 | 91 | ephrin-A1 (EFNA1) | 162997 | | SEQ ID No:136 | 0 | SEQ ID No:226 | SEQ ID No:227 |
| SELE | 92 | selectin E (endo- | 186132 | SEQ ID No:137 | SEQ ID No:138 | SEQ ID No:259 | SEQ ID No:260 | SEQ ID No:261 |
| | | thelial adhesion | | | | | | |
| | | molecule 1) (SELE) | | | | | | |
| APC | 93 | adenomatosis poly- | 125294 | SEQ ID NO:139 | SEQ ID No:140 | SEQ ID No:54 | SEQ ID No:55 | SEQ ID No:56 |
| | | posis coli (APC) | | | | | | |
| FAK | 94 | PTK2 protein tyro- | 195731 | | SEQ ID No:141 | 0 | SEQ ID No:284 | SEQ ID No:285 |
| | | sine kinase 2 | | | | | | |
| | | (PTK2) (ex FAK) | | | | | | |
| FOS-a | 95 | v-fos FBJ murine | 208717 | | SEQ ID No:142 | 0 | SEQ ID No:317 | SEQ ID No:318 |
| | | osteosarcoma | | | | | | |
| | | viral oncogene | | | | | | |
| | | homolog (FOS) | | | | | | |
| FGFR1 | 96 | fibroblast growth | 154472 | SEQ ID No:143 | SEQ ID No:144 | SEQ ID No:180 | SEQ ID No:181 | SEQ ID No:182 |
| | | factor receptor | | | | | | |
| | | (FGFr) | | | | | | |
| MC1R | 97 | melanocortin 1 re- | 155691 | | SEQ ID No:145 | 0 | SEQ ID No:187 | SEQ ID No:188 |
| | | ceptor (alpha | | | | | | |
| | | melanocyte stim- | | | | | | |
| | | ulating hormone | | | | | | |
| | | receptor) (MC1R) | | | | | | |
| PCNA | 98 | proliferating cell | 232941 | SEQ ID No:146 | SEQ ID No:147 | SEQ ID No:339 | SEQ ID No:340 | SEQ ID No:341 |
| | | nuclear antigen | | | | | | |
| | | (PCNA) | | | | | | |
| DDT | 99 | D-dopachrome tau- | 132109 | SEQ ID No:148 | SEQ ID No:149 | SEQ ID No:88 | SEQ ID No:89 | SEQ ID No:90 |
| | | tomerase (DDT) | | | | | | |
| GRB2 | 100 | growth factor re- | 172152 | SEQ ID No:150 | SEQ ID No:151 | SEQ ID No:230 | SEQ ID No:231 | SEQ ID No:232 |
| | | ceptor-bound | | | | | | |
| | | protein 2 (GRB2) | | | | | | |
| AMFR | 101 | autocrine motility | 146280 | SEQ ID No:152 | SEQ ID No:153 | SEQ ID No:111 | SEQ ID No:112 | SEQ ID No:113 |
| | | factor receptor | | | | | | |
| | | (AMFR) | | | | | | |
| ITGB2 | 102 | integrin, beta 2 | 187822 | SEQ ID No:154 | | 0 | SEQ ID No:267 | SEQ ID No:268 |
| | | 2 (antigen CD18 | | | | | | |
| | | (p95), lymphocyte | | | | | | |
| | | function-ass- | | | | | | |
| | | ociated antigen 1; | | | | | | |
| | | macrophage antigen | | | | | | |
| | | 1 (mac-1) beta | | | | | | |
| | | subunit) (ITGB2) | | | | | | |
| JUND | 103 | jun D proto- | 175421 | SEQ ID No:155 | | SEQ ID No:233 | 0 | SEQ ID No:234 |
| | | oncogene (JUND) | | | | | | |
| NF45 | 104 | interleukin en- | 243907 | | SEQ ID No:156 | 0 | SEQ ID No:350 | SEQ ID No:351 |
| | | hancer binding | | | | | | |
| | | factor 2 (ILF2) (ex | | | | | | |
| | | NF45) | | | | | | |
| PPP4C | 105 | protein phosphatase | 114097 | SEQ ID No:157 | SEQ ID No:158 | SEQ ID No:32 | SEQ ID No:33 | SEQ ID No:34 |
| | | 4 (formerly X) | | | | | | |
| | | (PPP4C) | | | | | | |
| EMS1 | 106 | ATX1 (antioxidant | 149172 | SEQ ID No:159 | | SEQ ID No:123 | SEQ ID No:124 | SEQ ID No:125 |
| | | protein 1, yeast) | | | | | | |
| | | homolog 1 | | | | | | |
| | | (ATOX1) (ex | | | | | | |
| | | EMS1) | | | | | | |
| BCL2 | 107 | B-cell CLL/lymph- | 147002 | SEQ ID No:160 | SEQ ID No:161 | SEQ ID No:115 | SEQ ID No:116 | SEQ ID No:117 |
| | | oma 2 (BCL2), nu- | | | | | | |
| | | clear gene encoding | | | | | | |
| | | mitochondrial pro- | | | | | | |
| | | tein, transcript var- | | | | | | |
| | | iant alpha | | | | | | |
| MGST1 | 108 | protein phosphatase | 182610 | SEQ ID No:162 | SEQ ID No:163 | SEQ ID No:248 | 0 | SEQ ID No:249 |
| | | 1, catalytic sub- | | | | | | |
| | | unit, alpha iso- | | | | | | |
| | | form (PPP1CA) (ex | | | | | | |
| | | MGST1) | | | | | | |
| PDGFRB | 109 | platelet-derived | 158976 | | SEQ ID No:164 | 0 | SEQ ID No:208 | SEQ ID No:209 |
| | | growth factor re- | | | | | | |
| | | ceptor, beta poly- | | | | | | |
| | | peptide (PDGFRB) | | | | | | |
| ANXA11 | 110 | annexin A11 | 158892 | | SEQ ID No:165 | 0 | SEQ ID No:206 | SEQ ID No:207 |
| | | (ANXA11) | | | | | | |
| GPX1 | 111 | histocompatability | 159809 | | SEQ ID No:166 | 0 | SEQ ID No:212 | SEQ ID No:213 |
| | | class II antigen | | | | | | |
| | | gamma chain | | | | | | |
| | | (CD74) (ex GPX1 | | | | | | |
| | | Glutation S trans- | | | | | | |
| | | férase) | | | | | | |
| CFR-1 | 112 | Golgi apparatus pro- | 153974 | SEQ ID No:167 | SEQ ID No:168 | SEQ ID No:173 | SEQ ID No:174 | SEQ ID No:175 |
| | | tein 1 (GLG1) (ex | | | | | | |
| | | CFR-1) | | | | | | |
| BTF3L3 | 113 | basic transcription | 195889 | SEQ ID No:169 | | SEQ ID No:289 | 0 | SEQ ID No:290 |
| | | factor 3 (BTF3) | | | | | | |
| EST R55460 | 114 | EST R55460 | 154997 | | SEQ ID No:170 | 0 | SEQ ID No:185 | 0 |
| AKT2 | 115 | v-akt murine thy- | 182552 | SEQ ID No:171 | | SEQ ID No:253 | 0 | SEQ ID No:254 |
| | | moma viral onco- | | | | | | |
| | | gene homolog 2 | | | | | | |
| | | (ATK2) | | | | | | |
| CDKN1A | 116 | cyclin-dependent | 152524 | SEQ ID No:172 | SEQ ID No:173 | SEQ ID No:144 | SEQ ID No:145 | SEQ ID No:146 |
| | | kinase inhibitor | | | | | | |
| | | (CDKN1A) | | | | | | |
| PPP2CA | 117 | protein phosphatase | 54685 | SEQ ID No:174 | SEQ ID No:175 | 0 | SEQ ID No:183 | SEQ ID No:184 |
| | | 2 (formerly 2A), | | | | | | |
| | | catalytic subunit, | | | | | | |
| | | alpha isoform | | | | | | |
| | | (PPP2CA) | | | | | | |
| MDM2 | 118 | mouse double min- | 148052 | SEQ ID No:176 | | 0 | SEQ ID No:120 | SEQ ID No:121 |
| | | ute 2, human homo- | | | | | | |
| | | logy of; p53-binding | | | | | | |
| | | protein (MDM2), | | | | | | |
| | | transcript variant | | | | | | |
| | | MDM2 | | | | | | |
| TNFRSF6 | 119 | tumor necrosis | 151767 | SEQ ID No:177 | SEQ ID No:178 | SEQ ID No:141 | SEQ ID No:142 | SEQ ID No:143 |
| | | factor receptor | | | | | | |
| | | superfamily, mem- | | | | | | |
| | | ber 6 (TNFRSF6) | | | | | | |
| CNTFR | 120 | ciliary neurotrophic | 156431 | | SEQ ID No:179 | 0 | SEQ ID No:192 | SEQ ID No:193 |
| | | factor receptor | | | | | | |
| | | (CNTFR) | | | | | | |
| JUNB | 121 | jun B proto-onco- | 153213 | SEQ ID No:180 | SEQ ID No:181 | SEQ ID No:153 | SEQ ID No:154 | SEQ ID No:155 |
| | | gene (JUNB) | | | | | | |
| CCND1 | 122 | cyclin D1 (PRAD1: | 110022 | SEQ ID No:182 | | SEQ ID No:9 | 0 | SEQ ID No:10 |
| | | parathyroid | | | | | | |
| | | adenomatosis 1) | | | | | | |
| | | (CCND1) | | | | | | |
| TDPX1 | 123 | peroxiredoxin 2 | 208439 | SEQ ID No:183 | SEQ ID No:184 | SEQ ID No:314 | SEQ ID No:315 | SEQ ID No:316 |
| | | (PRDX2) (ex | | | | | | |
| | | TDPX1) | | | | | | |
| GRB7 | 124 | growth factor | 130323 | SEQ ID No:185 | SEQ ID No:186 | SEQ ID No:79 | SEQ ID No:80 | SEQ ID No:81 |
| | | receptor-bound pro- | | | | | | |
| | | tein 7 (GRB7) | | | | | | |
| RBBP7 | 125 | retinoblastoma-bind- | 210874 | SEQ ID No:187 | SEQ ID No:188 | SEQ ID No:319 | SEQ ID No:320 | SEQ ID No:321 |
| | | ing protein 7 | | | | | | |
| | | (RBBP7) | | | | | | |
| TIMP1 | 126 | tissue inhibitor of | 162246 | SEQ ID No:190 | SEQ ID No:223 | SEQ ID No:224 | SEQ ID No:225 | SEQ ID NO:189 |
| | | metalloproteinase 1 | | | | | | |
| | | (erythyroid po- | | | | | | |
| | | tentiating act- | | | | | | |
| | | ivity, collagen- | | | | | | |
| | | ase inhibitor) | | | | | | |
| | | (TIMP1) | | | | | | |
| YES1 | 127 | v-yes-1 Yamaguchi | 204634 | SEQ ID No:191 | | SEQ ID No:303 | 0 | SEQ ID No:304 |
| | | sarcoma viral onco- | | | | | | |
| | | gene homolog 1 | | | | | | |
| | | (YES1) | | | | | | |
| RNF5 | 128 | ring finger protein | 112098 | | SEQ ID No:192 | 0 | SEQ ID No:25 | SEQ ID No:26 |
| | | 5 (RNF5) | | | | | | |
| PRKCSH | 129 | protein kinase C | 187232 | | SEQ ID No:193 | 0 | SEQ ID No:263 | SEQ ID No:264 |
| | | substrate 80K-H | | | | | | |
| | | (PRKCSH) | | | | | | |
| CTSD | 130 | cathepsin D (lyso- | 149401 | SEQ ID No:194 | SEQ ID No:195 | SEQ ID No:126 | SEQ ID No:127 | SEQ ID No:128 |
| | | somal aspartyl pro- | | | | | | |
| | | tease) (CTSD) | | | | | | |
| NEO1 | 131 | neogenin (chicken) | 188380 | | SEQ ID No:196 | 0 | SEQ ID No:269 | SEQ ID No:270 |
| | | homolog 1 (NEO1) | | | | | | |
| GAPD-a | 132 | glyceraldehyde-3- | 152847 | SEQ ID No:197 | | SEQ ID No:150 | SEQ ID No:151 | SEQ ID No:152 |
| | | phosphatase dehy- | | | | | | |
| | | drogenase (GAPD) | | | | | | |
| ACTG1 | 133 | actin, gamma 1 | 182291 | SEQ ID No:198 | SEQ ID No:199 | SEQ ID No:242 | SEQ ID No:243 | SEQ ID No:244 |
| | | (ACTG1) | | | | | | |
| ITGA6 | 134 | integrin, alpha 6 | 182431 | SEQ ID No:200 | SEQ ID No:201 | SEQ ID No:245 | SEQ ID No:246 | SEQ ID No:247 |
| | | (ITGA6) | | | | | | |
| GAPD-b | 135 | glyceraldehyde-3- | 153607 | SEQ ID No:202 | SEQ ID No:203 | SEQ ID No:166 | SEQ ID No:167 | SEQ ID No:152 |
| | | phosphate dehydro- | | | | | | |
| | | genase (GAPD) | | | | | | |
| ETV5-b | 136 | ets variant gene 5 | 203394 | SEQ ID No:204 | SEQ ID No:205 | SEQ ID No:298 | SEQ ID No:299 | SEQ ID No:300 |
| | | (ets-related mole- | | | | | | |
| | | cule) (ETV5) | | | | | | |
| CDK4-b | 137 | cyclin-dependent | 195800 | SEQ ID No:206 | SEQ ID No:207 | SEQ ID No:286 | SEQ ID No:287 | SEQ ID No:288 |
| | | kinase 4 (CDK4) | | | | | | |
| FOS-b | 138 | v-fos FBJ murine | 363796 | SEQ ID No:208 | SEQ ID No:209 | SEQ ID No:404 | SEQ ID No:405 | SEQ ID No:318 |
| | | osteosarcoma viral | | | | | | |
| | | oncogene homo- | | | | | | |
| | | log (FOS) | | | | | | |
| HOXA5 | 139 | homebox protein | 300564 | SEQ ID No:210 | SEQ ID No:211 | SEQ ID No:382 | SEQ ID No:383 | SEQ ID No:384 |
| | | (HOX-1.3) (ex Hox | | | | | | |
| | | A5) | | | | | | |
| RELA | 140 | NF-kappa-B trans- | 122056 | SEQ ID No:212 | | SEQ ID No:42 | 0 | SEQ ID No:43 |
| | | cription factor p65 | | | | | | |
| | | DNA binding sub- | | | | | | |
| | | unit (ex RELa) | | | | | | |
| SUI1 | 141 | S100 calcium-bind- | 155345 | SEQ ID No:213 | SEQ ID No:214 | SEQ ID No:186 | 0 | 0 |
| | | ing protein A11 | | | | | | |
| | | (calgizzarin) | | | | | | |
| | | (S100A11) | | | | | | |
| ANG | 142 | angiogenin, ribonu- | 156720 | | SEQ ID No:215 | 0 | SEQ ID N:194 | SEQ ID No:195 |
| | | clease, RNase | | | | | | |
| | | A family, 5 | | | | | | |
| | | (ANG) | | | | | | |
| ITGA6 | 143 | integrin, alpha 6 | 182431 | SEQ ID No:216 | SEQ ID No:217 | SEQ ID No:245 | SEQ ID No:246 | SEQ ID No:247 |
| | | (ITGA6) | | | | | | |
| PRMT2 | 144 | HMT1 (hnRNP | 158038 | SEQ ID No:218 | SEQ ID No:219 | SEQ ID No:201 | SEQ ID No:202 | SEQ ID No:203 |
| | | methyltransfer- | | | | | | |
| | | ase, S. cerevis- | | | | | | |
| | | iae)-like 1 | | | | | | |
| | | (HRMTIL1) (ex | | | | | | |
| | | PRMT2) | | | | | | |
| EST R55460 | 145 | EST R55460 | 154997 | | SEQ ID No:220 | 0 | SEQ ID No:185 | 0 |
| GZMA | 146 | granzyme A (gran- | 356763 | SEQ ID No:221 | SEQ ID No:222 | SEQ ID No:402 | 0 | SEQ ID No:403 |
| | | zyme 1, cytotoxic | | | | | | |
| | | T-lymphocyte-ass- | | | | | | |
| | | ociated serine es- | | | | | | |
| | | terase 3) (GZMA) | | | | | | |
| SOX9 | 147 | SRY (sex-deter- | 323948 | SEQ ID No:223 | | SEQ ID No:394 | 0 | SEQ ID No:395 |
| | | mining region Y)- | | | | | | |
| | | box 9 (campomel- | | | | | | |
| | | ic dysplasia, auto- | | | | | | |
| | | somal sex-reversal) | | | | | | |
| | | (SOX9) | | | | | | |
| SRF | 148 | serum response | 321329 | | SEQ ID No:224 | SEQ ID No:391 | SEQ ID No:392 | SEQ ID No:393 |
| | | factor (c-fos serum | | | | | | |
| | | response element- | | | | | | |
| | | binding transcription | | | | | | |
| | | factor) (SRF) | | | | | | |
| EDNI | 149 | endothelial 1 | 153424 | SEQ ID No:225 | | #N/A | #N/A | #N/A |
| | | (EDN1) | | | | | | |
| PTPN6 | 150 | protein tyrosine | 66778 | SEQ ID No:226 | | #N/A | #N/A | #N/A |
| | | phosphatase, non- | | | | | | |
| | | receptor type 6 | | | | | | |
| | | (PTPN6) | | | | | | |
| TFAP4 | 151 | transcription factor | 159093 | SEQ ID No:227 | | 0 | SEQ ID No:210 | SEQ ID No:211 |
| | | AP-4 (activating | | | | | | |
| | | enhancer bind- | | | | | | |
| | | ing protein 4) | | | | | | |
| | | (TFAP4) | | | | | | |
| ELF1 | 152 | Human cis-acting- | 182007 | SEQ ID No:228 | | SEQ ID No:417 | 0 | 0 |
| | | sequence Elf-1 | | | | | | |
| CD2 | 153 | CD2 antigen (p50), | 120649 | SEQ ID No:229 | | SEQ ID No:431 | 0 | 0 |
| | | sheep red blood | | | | | | |
| | | cell receptor | | | | | | |
| | | (CD2) | | | | | | |
| CCND2 | 154 | cyclin D2 (CCND2) | 175256 | SEQ ID No:230 | | #N/A | #N/A | #N/A |
| IL3RA | 155 | interleukin 3 recep- | 183087 | SEQ ID No:231 | | SEQ ID No:440 | SEQ ID No:441 | 0 |
| | | tor (hIL-3Ra) | | | | | | |
| JUP | 156 | junction plakoglobin | 157958 | SEQ ID No:232 | | #N/A | #N/A | #N/A |
| | | (JUP) | | | | | | |
| RBL2 | 157 | retinoblastoma-like | 108571 | SEQ ID No:233 | | SEQ ID No:430 | 0 | 0 |
| | | 2 (p130) (RBL2) | | | | | | |
| HOXA4 | 158 | homeo box A4 | 110731 | SEQ ID No:234 | | SEQ ID No:20 | SEQ ID No:21 | 0 |
| | | (HOXA4) | | | | | | |
| ACY1 | 159 | aminoacylase | 160764 | SEQ ID No:235 | | SEQ ID No:435 | SEQ ID No:436 | 0 |
| | | (ACY1) | | | | | | |
| GADD45A | 160 | growth arrest and | 115176 | SEQ ID No:236 | | #N/A | #N/A | #N/a |
| | | DNA-damage-in- | | | | | | |
| | | ducible, alpha | | | | | | |
| | | (GADD45A) | | | | | | |
| nm23 | 161 | non-metastatic | 174388 | SEQ ID No:237 | | #N/A | #N/A | #N/A |
| | | cells 1, protein | | | | | | |
| | | (NM23A) express- | | | | | | |
| | | ed (NME1) | | | | | | |
| BBC1 | 162 | ribosomal protein | 178317 | SEQ ID No:238 | | #N/A | #N/A | #N/A |
| | | L13 (RPL13) (ex | | | | | | |
| | | BBC1) | | | | | | |
| VEGFB | 163 | vascular endothe- | 162499 | SEQ ID No:239 | | #N/A | #N/A | #N/A |
| | | lial growth factor B | | | | | | |
| | | (VEGFB) | | | | | | |
| LAMR1 | 164 | laminin receptor 1 | 199837 | SEQ ID No:240 | | #N/A | #N/A | #N/A |
| | | (67kD, ribosomal | | | | | | |
| | | protein SA) | | | | | | |
| | | (LAMR1) | | | | | | |
| IL2RB | 165 | interleukin 2 re- | 139073 | SEQ ID No:241 | SEQ ID No:242 | SEQ ID No:97 | SEQ ID No:98 | SEQ ID No:99 |
| | | ceptor, beta | | | | | | |
| | | (IL2RB) | | | | | | |
| DES | 166 | desmin | 153854 | SEQ ID No:243 | | SEQ ID No:168 | SEQ ID No:169 | SEQ ID No:170 |
| PRL | 167 | prolactin | 133738 | SEQ ID No:244 | | SEQ ID No:91 | SEQ ID No:92 | SEQ ID No:93 |
| CSH1 | 168 | Chorionic soma- | 133891 | | SEQ ID No:245 | SEQ ID No:432 | 0 | 0 |
| | | tomammotropin hor- | | | | | | |
| | | mone 1 (placental | | | | | | |
| | | lactogen) = LAC- | | | | | | |
| | | TOGEN Precursor | | | | | | |
| TEK | 169 | tyrosine proteine | 151501 | SEQ ID No:246 | SEQ ID No:247 | SEQ ID No:138 | SEQ ID No:139 | SEQ ID No:140 |
| | | kinase receptor | | | | | | |
| Nrg1 | 170 | neuregulin 1 (EST | 155716 | SEQ ID No:248 | SEQ ID No:249 | SEQ ID No:189 | SEQ ID No:190 | SEQ ID No:191 |
| | | R72075) | | | | | | |
| PLAT | rien | pas dEST ni | 160149 | | | SEQ ID No:433 | SEQ ID No:434 | 0 |
| | | mRNA | | | | | | |
| EST | rien | | image ? | | | | | |
| AW184517 | | | | | | | | |

Tables 5 hereunder displays subpopulations of polynucleotide sequences interesting to distinguish a person without cancer from a cancer patient.

TABLE 5

| Gene | | | | | |
|---|---|---|---|---|---|
| symbol | No | Name | Seq3′ | Seq5′ | Ref |
| HRB | 1 | hiv-1 rev binding protein | SEQ ID | | SEQ ID |
| | | | No:1 | | No:2 |
| EST T81919 | 4 | ests, weakly similar to alu7_human alu subfamily | SEQ ID | SEQ ID | |
| | | sq sequence contamination warning entry [h. sapines] | No:7 | No:8 | |
| ENPP2 | 18 | ectonucleotide pyrophosphatase/phosphodiesterase 2 | SEQ ID | SEQ ID | SEQ ID |
| | | (autotaxin) | No:39 | No:40 | No:41 |
| TNXB | 21 | tenascin xb | | SEQ ID | SEQ ID |
| | | | | No:46 | No:47 |
| APC | 24 | adenomatosis polyposis coli | SEQ ID | SEQ ID | SEQ ID |
| | | | No:54 | No:55 | NO:56 |
| GATA3 | 32 | gata-binding protein 3 | SEQ ID | SEQ ID | SEQ ID |
| | | | No:76 | No:77 | No:78 |
| PRL | 38 | prolactin | SEQ ID | SEQ ID | SEQ ID |
| | | | No:91 | No:92 | No:93 |
| BCL2 | 48 | b-cell cll/lymphoma 2 | SEQ ID | SEQ ID | SEQ ID |
| | | | No:115 | No:116 | No:117 |
| CTSD | 53 | cathepsin d (lysosomal aspartyl protease) | SEQ ID | SEQ ID | SEQ ID |
| | | | No:126 | No:127 | No:128 |
| TEK | 58 | tek tyrosine kinase, endothelial (venous | SEQ ID | SEQ ID | SEQ ID |
| | | malformations, multiple cutaneous and mucosal) | No:138 | No:139 | No:140 |
| TNFRSF6 | 59 | tumor necrosis factor receptor superfamily, member | SEQ ID | SEQ ID | SEQ ID |
| | | 6 | No:141 | No:142 | No:143 |
| PLA2G2A | 61 | phospholipase a2, group iia (platelets, synovial | SEQ ID | SEQ ID | SEQ ID |
| | | fluid | No:147 | No:148 | No:149 |
| CRABP2 | 64 | cellular retinoic acid-binding protein 2 | SEQ ID | SEQ ID | SEQ ID |
| | | | No:156 | No:157 | No:158 |
| RIL | 66 | lim domain protein | SEQ ID | SEQ ID | SEQ ID |
| | | | | No:162 | No:163 |
| DES | 69 | desmin | SEQ ID | SEQ ID | SEQ ID |
| | | | No:168 | No:169 | No:170 |
| GZMB | 73 | granzyme b (granzyme 2, cytotoxic t-lymphocyte- | SEQ ID | | SEQ ID |
| | | associated serine esterase 1) | No:178 | | No:179 |
| ETV4 | 85 | ets variant gene 4 (e1a enhancer-binding protein, | SEQ ID | SEQ ID | |
| | | e1af) | No:204 | No:205 | |
| WBSCR14 | 88 | williams-beuren syndrome chromosome region 14 | | SEQ ID | SEQ ID |
| | | | | No:210 | No:211 |
| THBS1 | 91 | thrombospondin 1 | SEQ ID | | SEQ ID |
| | | | No:216 | | No:217 |
| GRB2 | 97 | growth factor receptor-bound protein 2 | SEQ ID | SEQ ID | SEQ ID |
| | | | No:230 | No:231 | No:232 |
| RAD9 | 104 | rad9 (s. pombe) homolog | SEQ ID | | SEQ ID |
| | | | No:248 | | No:249 |
| ATF3 | 105 | activating transcription factor 3 | SEQ ID | SEQ ID | SEQ ID |
| | | | No:250 | No:251 | No:252 |
| DTR | 112 | diphtheria receptor (heparin-binding epidermal | | SEQ ID | SEQ ID |
| | | growth factor-like growth factor) | | No:265 | No:266 |
| ITGB2 | 113 | integrin, beta 2 (antigen cd18 (p95), lymphocyte | | SEQ ID | SEQ ID |
| | | function-associated antigen 1, macrophage entigen 1 | | No:267 | No:268 |
| | | (mac-1) beta subunit) | | | |
| POU2F2 | 115 | pou domain, class 2, transcription factor 2 | SEQ ID | | SEQ ID |
| | | | No:271 | | No:272 |
| MYBL2 | 131 | v-myb avian myeoblastosis viral oncogene | SEQ ID | SEQ ID | SEQ ID |
| | | homolog-like 2 | No:308 | No:309 | No:310 |
| TGFBR3 | 132 | transforming growth factor, beta receptor iii | SEQ ID | SEQ ID | SEQ ID |
| | | (betaglycan, 300kd) | No:311 | No:312 | No:313 |
| FOS | 134 | v-fos fbj murine osteosarcoma viral oncogene | | SEQ ID | SEQ ID |
| | | homolog | | No:317 | No:318 |
| ABCC5 | 137 | atp-binding cassette, sub-family c (cftr/mrp), | | SEQ ID | SEQ ID |
| | | member 5 | | No:324 | No:325 |
| MMP11 | 145 | matrix metalloproteinase 11 (stromelysin 3) | SEQ ID | | SEQ ID |
| | | | No:345 | | No:346 |
| ILF2 | 147 | interleukin enhancer binding factor 2, 45kd | | SEQ ID | SEQ ID |
| | | | | No:350 | No:351 |
| ETV5 | 155 | ets variant gene 5 (ets-related molecule) | SEQ ID | SEQ ID | SEQ ID |
| | | | No:368 | No:369 | No:300 |
| RELB | 175 | v-rel avian reticuloendotheliosis viral oncogene | SEQ ID | SEQ ID | SEQ ID |
| | | homolog b (nuclear factor of kappa light polypeptide | No:417 | No:418 | No:419 |
| | | gene enhancer in b-cells 3) | | | |
| EST T80406 | 180 | similar to SP:S36648 S36648 RB2/P130 PROTEIN | SEQ ID | | |
| | | | No:430 | | |
| EST Y95640 | 181 | similar to gb:M16336 T-CELL SURFACE | SEQ ID | | |
| | | ANTIGEN CD2 | No:431 | | |
| EST R28523 | 182 | similar to placental lactogen (CSH1) | SEQ ID | | |
| | | | No:432 | | |
| EST H28056 | 185 | Homo sapines E74-like factor 1 (ets domain | SEQ ID | | |
| | | transcription factor) (ELF1) | No:437 | | |
| ESTs H42957 & | 187 | Human interleukin 3 receptor (hIL-3Ra) | SEQ ID | SEQ ID | |
| H42888 | | | No:440 | No:441 | |

Tables 5A and 5B hereunder displays two subpopulations corresponding to the 5 top overexpressed and to the 5 top underexpressed polynucleotide sequences particularly interesting to distinguish a person without cancer from a cancer patient.

TABLE 5A
overexpressed genes:top 5

| Gene | | | | | |
|---|---|---|---|---|---|
| symbol | No | Name | Seq3′ | Seq5′ | Ref |
| GATA3 | 32 | gata-binding protein 3 | SEQ ID | SEQ ID | SEQ ID |
| | | | No:76 | No:77 | No:78 |
| GZMB | 73 | granzyme b (granzyme 2, cytotoxic t- | SEQ ID | | SEQ ID |
| | | lymphocyte-associated serine esterase 1) | No:178 | | No:179 |
| MYBL2 | 131 | v-myb avian myeloblastosis viral oncogene | SEQ ID | SEQ ID | SEQ ID |
| | | homolog-like 2 | No:308 | No:309 | No:310 |
| MMP11 | 145 | matrix metallopropteinase 11 (stromelysin 3) | SEQ ID | | SEQ ID |
| | | | No:345 | | No:346 |
| EST | 181 | similar to gb:M16336 T-CELL SURFACE | SEQ ID | | |
| T95640 | | ANTIGEN CD2 | No:431 | | |

TABLE 5B
underexpressed genes:top 5

| Gene | | | | | |
|---|---|---|---|---|---|
| symbol | No | Name | Seq3′ | Seq5′ | Ref |
| PRL | 38 | prolactin | SEQ ID | SEQ ID | SEQ ID |
| | | | No:91 | No:92 | No:93 |
| TEK | 58 | tek tyrosine kinase, endothelial (venous | SEQ ID | SEQ ID | SEQ ID |
| | | malformations, multiple cutaneous and mucosal) | No:138 | No:139 | No:140 |
| PLA2GA | 612 | phospholipase a2, group iia (platelets, synovial fluid) | SEQ ID | SEQ ID | SEQ ID |
| | | | No:147 | No:148 | No:149 |
| DES | 69 | desmin | SEQ ID | SEQ ID | SEQ ID |
| | | | No:168 | No:169 | No:170 |
| EST R28523 | 182 | similar to placental lactogen (CSH1) | SEQ ID | | |
| | | | No:432 | | |

Table 6 hereunder relates to subpopulations of polynucleotide sequences interesting to detect hormone-sensitive tumors allowing distinction between ER+ and ER-samples.

TABLE 6

| Gene | | | | | |
|---|---|---|---|---|---|
| symbol | No | Name | Seq3′ | Seq5′ | Ref |
| SOX4 | 11 | sry (sex determining region y)-box 4 | SEQ ID | SEQ ID | SEQ ID |
| | | | No:22 | No:23 | No:24 |
| IGF2 | 26 | insulin-like growth factor 2 (somatomedian a) | SEQ ID | SEQ ID | SEQ ID |
| | | | No:59 | No:60 | No:61 |
| GATA3 | 32 | gata-binding protein 3 | SEQ ID | SEQ ID | SEQ ID |
| | | | No:76 | No:77 | No:78 |
| TOP2B | 34 | topoisomerase (dna) ii beta (180kd) | | SEQ ID | SEQ ID |
| | | | | No:82 | No:83 |
| IL2RB | 40 | interleukin 2 receptor, beta | SEQ ID | SEQ ID | SEQ ID |
| | | | No:97 | No:98 | No:99 |
| EGFR | 57 | epidermal growth factor receptor (avian | SEQ ID | SEQ ID | SEQ ID |
| | | erythroblastic leukemia viral (v-erb-b) oncogene | No:135 | No:136 | No:137 |
| | | homolog) | | | |
| CRABP2 | 64 | cellular retinoic acid-binding protein 2 | SEQ ID | SEQ ID | SEQ ID |
| | | | No:156 | No:157 | No:158 |
| S100B | 107 | s100 calcium-binding protein, beta (neural) | | SEQ ID | SEQ ID |
| | | | | No:255 | No:256 |
| IL2RG | 119 | interleukin 2 receptor, gamma (severe combined | SEQ ID | SEQ ID | SEQ ID |
| | | immunodeficiency) | No:279 | No:280 | No:281 |
| KIAA1075 | 136 | kiaa 1075 protein | SEQ ID | SEQ ID | |
| | | | No:322 | No:323 | |
| MST1 | 140 | macrophage stimulating 1 (hepatocyte growth factor- | SEQ ID | SEQ ID | SEQ ID |
| | | like) | No:331 | No:332 | No:333 |
| GSTP1 | 141 | glutathione s-transferase pi | SEQ ID | SEQ ID | SEQ ID |
| | | | No:334 | No:335 | No:336 |
| MMP11 | 145 | matrix metalloproteinase 11 (stromelysin 3) | SEQ ID | | SEQ ID |
| | | | No:345 | | No:346 |
| FLJ11307 | 148 | hypothetical protein flj11307 | SEQ ID | | SEQ ID |
| | | | No:352 | | No:353 |
| MYB | 149 | v-myb avian myeloblastosis viral oncogene homolog | | SEQ ID | SEQ ID |
| | | | | No:354 | No:355 |
| XBP1 | 162 | x-box binding protein 1 | SEQ ID | SEQ ID | SEQ ID |
| | | | No:385 | No:386 | No:387 |
| SOX9 | 165 | sry (sex dtermining region y)-boc 9 (campomelic | SEQ ID | | SEQ ID |
| | | dysplasia, autosomal sex-reversal) | No:394 | | No:395 |
| GZMA | 169 | granzyme a (granzyme 1, cytotoxic t-lymphocyte- | SEQ ID | | SEQ ID |
| | | associated serine esterase 3) | No:402 | | No:403 |
| CD3G | 174 | cd3g antigen, gamma polypeptide (tit3 complex) | SEQ ID | SEQ ID | SEQ ID |
| | | | No:414 | No:415 | No:416 |
| EST | 188 | Human tumor protein p53 (Li-Fraumeni syndrome) | SEQ ID | | |
| H57912 | | (TP53) | No:442 | | |

Tables 6A and 6B hereunder relate to two subpopulations of polynucleotide sequences particularly interesting to detect hormone-sensitive tumors allowing distinction between ER+ and ER− samples

TABLE 6A
overexpressed genes:top 5
ER +/ER −

| Gene | CL | | | | |
|---|---|---|---|---|---|
| symbol | No | Name | Seq3′ | Seq5′ | Ref |
| GATA3 | 32 | gata-binding protein 3 | SEQ ID | SEQ ID | SEQ ID |
| | | | No:76 | No:77 | No:78 |
| KIAA1075 | 136 | kiaa 1075 protein | SEQ ID | SEQ ID | |
| | | | No:322 | No:323 | |
| MMP11 | 145 | matrix metalloproteinase 11 | SEQ ID | | SEQ ID |
| | | (stromelysin 3) | No:345 | | No:346 |
| MYB | 149 | v-myb avian myeloblastosis viral | | SEQ ID | SEQ ID |
| | | oncogene homolog | | No:354 | No:355 |
| GZMA | 169 | granzyme a (granzyme 1, yutotoxic t- | SEQ ID | | SEQ ID |
| | | lymphocyte-associated serine esterase 3) | No:402 | | No:403 |

TABLE 6B
underexpressed genes:top 5

| Gene | | | | | |
|---|---|---|---|---|---|
| symbol | No | Name | Seq3′ | Seq5′ | Ref |
| SOX4 | 11 | sry (sex determining region y)-box 4 | SEQ ID | SEQ ID | SEQ ID |
| | | | No:22 | No:23 | No:24 |
| IL2RB | 40 | interleukin 2 receptor, beta | SEQ ID | SEQ ID | SEQ ID |
| | | | No:97 | No:98 | No:99 |
| EGFR | 57 | epidermal growth factor receptor (avian | SEQ ID | SEQ ID | SEQ ID |
| | | eryhtroblastic leukemia viral (v-erb-b) | No:135 | No:136 | No:137 |
| | | oncogene homolog) | | | |
| IL2RG | 119 | interleukin 2 receptor, gamma (severe | SEQ ID | SEQ ID | SEQ ID |
| | | combined immunodeficiency) | No:279 | No:280 | No:281 |
| CD3G | 174 | cd3g antigen, gamma polypeptide (tit3 | SEQ ID | SEQ ID | SEQ ID |
| | | complex) | No:414 | No:415 | No:416 |

Tables 7 hereunder relates to subpopulations of polynucleotide sequences interesting to distinguish tumors in which a lymph node has been invaded by a tumor cell from tumors in which a lymph node has not been so invaded.

TABLE 7

| Gene | CL | | | | |
|---|---|---|---|---|---|
| symbol | No | Name | Seq3′ | Seq5′ | Ref |
| EST T89980 | 8 | ests | SEQ ID | | |
| | | | No:16 | | |
| SOX4 | 11 | sry (sex determining region y)-box 4 | SEQ ID | SEQ ID | SEQ ID |
| | | | No:22 | No:23 | No:24 |
| ENPP2 | 18 | ectonucleotide | SEQ ID | SEQ ID | SEQ ID |
| | | pyrophosphatase/phosphodiesterase 2 | No:39 | No:40 | No:41 |
| | | (autotoxin) | | | |
| MUC1 | 25 | mucin 1, transmembrane | | SEQ ID | SEQ ID |
| | | | | No:57 | No:58 |
| GATA3 | 32 | gata-binding protein 3 | SEQ ID | SEQ ID | SEQ ID |
| | | | No:76 | No:77 | No:78 |
| TOP2B | 34 | topoisomerase (dna) it beta (180kd) | | SEQ ID | SEQ ID |
| | | | | No:82 | No:83 |
| IL2RB | 40 | interleukin 2 receptor, beta | SEQ ID | SEQ ID | SEQ ID |
| | | | No:97 | No:98 | No:99 |
| ERBB2 | 49 | v-erb-b2 avian erythroblastic | | SEQ ID | SEQ ID |
| | | leukemia viral oncogene homolog 2 | | No:118 | No:119 |
| | | (neuro/glioblastoma derived oncogene | | | |
| | | homolog) | | | |
| EGFR | 57 | epidermal growth factor receptor (avian | SEQ ID | SEQ ID | SEQ ID |
| | | erythroblastic leukemia viral (v-erb-b) | No:135 | No:136 | No:137 |
| | | oncogene homolog) | | | |
| THBS1 | 91 | thrombospondin 1 | SEQ ID | | SEQ ID |
| | | | No:216 | | No:217 |
| PPP2R2C | 100 | protein phosphatase 2 (formerly 2a), | SEQ ID | SEQ ID | |
| | | regulatory subunit b (pr 52), gamma | No:238 | No:239 | |
| | | isoform | | | |
| ATF3 | 105 | activating transcription factor 3 | SEQ ID | SEQ ID | SEQ ID |
| | | | No:250 | No:251 | No:252 |
| KIAA1075 | 136 | kiaa 1075 protein | SEQ ID | SEQ ID | |
| | | | No:322 | No:323 | |
| CDH1 | 138 | cadherin 1, type 1, e-cadherin (epithelial) | SEQ ID | SEQ ID | SEQ ID |
| | | | No:326 | No:327 | No:328 |
| ZNF144 | 139 | zinc finger protein 144 (mel-18) | | SEQ ID | SEQ ID |
| | | | | No:329 | No:330 |
| GSTP1 | 141 | glutathione s-transferase pi | SEQ ID | SEQ ID | SEQ ID |
| | | | No:334 | No:335 | No:336 |
| CD44 | 158 | cd44 antigen (homing function and indian | SEQ ID | SEQ ID | SEQ ID |
| | | blood group system) | No:374 | No:375 | No:376 |
| GZMA | 169 | granzyme a (granzyme 1, cytotoxic t-lym- | SEQ ID | | SEQ ID |
| | | phocyte-associated serine esterase 3) | No:402 | | No:403 |
| EST T80406 | 180 | similar to SP;S36648 S36648 RB2/P130 | SEQ ID | | |
| | | PROTEIN | No:430 | | |
| ESTs H30141 & | 186 | Homo sapiens selectin P | SEQ ID | SEQ ID | |
| H27466 | | | No:438 | No:439 | |

Tables 7A and 7B hereunder relate to two subpopulations of polynucleotide sequences particularly interesting to distinguish tumors in which a lymph node has been invaded by a tumor cell from tumors in which a lymph node has not been so invaded.

TABLE 7A
Overexpressed genes:top 5

| Gene | | | | | |
|---|---|---|---|---|---|
| symbol | No | Name | Seq3′ | Seq5′ | Ref |
| ENPP2 | 18 | ectonucleotide | SEQ ID | SEQ ID | SEQ ID |
| | | pyrophosphatase/phosphodiesterase 2 | No:39 | No:40 | No:41 |
| | | (autotaxin) | | | |
| GATA3 | 32 | gata-binding protein 3 | SEQ ID | SEQ ID | SEQ ID |
| | | | No:76 | No:77 | No:78 |
| EGFR | 57 | epidermal growth factor receptor (avian | SEQ ID | SEQ ID | SEQ ID |
| | | erythroblastic leukemia viral (v-erb-b) | No:135 | No:136 | No:137 |
| | | oncogene homolog) | | | |
| THBS1 | 91 | thrombospondin 1 | SEQ ID | | SEQ ID |
| | | | No:216 | | No:217 |
| ATF3 | 105 | activating transcription factor 3 | SEQ ID | SEQ ID | SEQ ID |
| | | | No:250 | No:251 | No:252 |

TABLE 7B
Underexpressed genes: top 5

| Gene | | | | | |
|---|---|---|---|---|---|
| symbol | No | Name | Seq 3′ | Seq 5′ | Ref |
| SOX4 | 11 | sry (sex determining region y)-box 4 | SEQ ID | SEQ ID | SEQ ID |
| | | | No: 22 | No: 23 | No: 24 |
| IL2RB | 40 | interleukin 2 receptor, beta | SEQ ID | SEQ ID | SEQ ID |
| | | | No: 97 | No: 98 | No: 99 |
| ERBB2 | 49 | v-erb-b2 avian erythroblastic leukemia | | SEQ ID | SEQ ID |
| | | viral oncogene homolog 2 | | No: 118 | No: 119 |
| | | (neuro/glioblastoma derived oncogene | | | |
| | | homolog) | | | |
| PPP2R2C | 100 | protein phosphatase 2 (formerly 2a), | SEQ ID | SEQ ID | |
| | | regulatory subunit b (pr 52), gamma | No: 238 | No: 239 | |
| | | isoform | | | |
| GSTP1 | 141 | glutathione s-transferase pi | SEQ ID | SEQ ID | SEQ ID |
| | | | No: 334 | No: 335 | No:336 |

Table 8 hereunder relates to subpopulations of polynucleotide sequences particularly interesting to distinguish tumors sensitive to anthracycline from tumors insensitive to anthracycline.

TABLE 8
A1/A2

| Gene | | | | | |
|---|---|---|---|---|---|
| symbol | No | Name | Seq 3′ | Seq 5′ | Ref |
| SOX4 | 11 | sry (sex determining region y)-box | SEQ ID | SEQ ID | SEQ ID |
| | | | No: 22 | No: 23 | No: 24 |
| CSF1 | 22 | colony stimulating factor 1 (macrophage) | SEQ ID | SEQ ID | SEQ ID |
| | | | No: 48 | No: 49 | No: 50 |
| VIL2 | 23 | villin 2 (ezrin) | SEQ ID | SEQ ID | SEQ ID |
| | | | No: 51 | No: 52 | No: 53 |
| IGF2 | 26 | insulin-like growth factor 2 (somatomedin a) | SEQ ID | SEQ ID | SEQ ID |
| | | | No: 59 | No: 60 | No: 61 |
| KIAA0427 | 28 | kiaa0427 gene product | SEQ ID | SEQ ID | SEQ ID |
| | | | No: 65 | No: 66 | No: 67 |
| MYC | 31 | v-myc avian myelocytomatosis viral oncogene | SEQ ID | SEQ ID | SEQ ID |
| | | homolog | No: 73 | No: 74 | No: 75 |
| GATA3 | 32 | gata-binding protein 3 | SEQ ID | SEQ ID | SEQ ID |
| | | | No: 76 | No: 77 | No: 78 |
| TOP2B | 34 | topoisomerase (dna) ii beta (180 kd) | | SEQ ID | SEQ ID |
| | | | | No: 82 | No: 83 |
| ERBB2 | 49 | v-erb-b2 avian erythroblastic leukemia viral | | SEQ ID | SEQ ID |
| | | oncogene homolog 2 (neuro/glioblastoma | | No: 118 | No: 119 |
| | | derived oncogene homolog) | | | |
| EGFR | 57 | epidermal growth factor receptor (avian | SEQ ID | SEQ ID | SEQ ID |
| | | erythroblastic leukemia viral (v-erb-b) | No: 135 | No: 136 | No: 137 |
| | | oncogene homolog) | | | |
| CRABP2 | 64 | cellular retinoic acid-binding protein 2 | SEQ ID | SEQ ID | SEQ ID |
| | | | No: 156 | No: 157 | No: 158 |
| GZMB | 73 | granzyme b (granzyme 2, cytotoxic t- | SEQ ID | | SEQ ID |
| | | lymphocyte-associated serine esterase 1) | No: 178 | | No: 179 |
| IGKC | 77 | immunoglobulin kappa constant | SEQ ID | | |
| | | | No: 186 | | |
| ANG | 81 | angiogenin, ribonuclease, rnase a family, 5 | SEQ ID | SEQ ID | |
| | | | No: 194 | No: 195 | |
| EFNA1 | 95 | ephrin-al | | SEQ ID | SEQ ID |
| | | | | No: 226 | No: 227 |
| MYBL2 | 131 | v-myb avian myeloblastosis viral oncogene | SEQ ID | SEQ ID | SEQ ID |
| | | homolog-like 2 | No: 308 | No: 309 | No: 310 |
| CDH1 | 138 | cadherin 1, type 1, e-cadherin (epithelial) | SEQ ID | SEQ ID | SEQ ID |
| | | | No: 326 | No: 327 | No: 328 |
| MST1 | 140 | macrophage stimulating 1 (hepatocyte growth | SEQ ID | SEQ ID | SEQ ID |
| | | factor-like) | No: 331 | No: 332 | No: 333 |
| MYB | 149 | v-myb avian myeloblastosis viral oncogene | | SEQ ID | SEQ ID |
| | | homolog | | No: 354 | No: 355 |
| XBP1 | 162 | x-box binding protein 1 | SEQ ID | SEQ ID | SEQ ID |
| | | | No: 385 | No: 386 | No: 387 |
| SRF | 164 | serum response factor (c-fos serum response | SEQ ID | SEQ ID | SEQ ID |
| | | element-binding transcription factor) | No: 391 | No: 392 | No: 393 |
| SOX9 | 165 | sry (sex determining region y)-box 9 | SEQ ID | | SEQ ID |
| | | (campomelic dysplasia, autosomal sex-reversal) | No: 394 | | No: 395 |
| ESTs H21879 | 183 | Homo sapiens plasminogen activator (PLAT) | SEQ ID | SEQ ID | |
| & H21880 | | | No: 433 | No: 434 | |

Tables 8A and 8B hereunder relate to two subpopulations of polynucleotide sequences particularly interesting to distinguish tumors sensitive to anthracycline from tumors insensitive to anthracycline.

TABLE 8A
Overexpressed genes: top 5

| Gene | | | | | |
|---|---|---|---|---|---|
| symbol | No | Name | Seq 3′ | Seq 5′ | Ref |
| GATA3 | 32 | gata-binding protein 3 | SEQ ID | SEQ ID | SEQ ID |
| | | | No: 76 | No: 77 | No: 78 |
| KIAA1075 | 136 | kiaa1075 protein | SEQ ID | SEQ ID | |
| | | | No: 322 | No: 323 | |
| MMP11 | 145 | matrix metalloproteinase 11 | SEQ ID | | SEQ ID |
| | | (stromelysin 3) | No: 345 | | No: 346 |
| MYB | 149 | v-myb avian myeloblastosis viral | | SEQ ID | SEQ ID |
| | | oncogene homolog | | No: 354 | No: 355 |
| GZMA | 169 | Granzyme a (granzyme 1, cytotoxic t- | SEQ ID | | SEQ ID |
| | | lymphocyte-associated serine esterase 3) | No: 402 | | No: 403 |

TABLE 8B
underexpressed genes: top 5

| Gene | | | | | |
|---|---|---|---|---|---|
| symbol | No | Name | Seq 3′ | Seq 5′ | Ref |
| SOX4 | 11 | sry (sex determining region y)-box 4 | SEQ ID | SEQ ID | SEQ ID |
| | | | No: 22 | No: 23 | No: 24 |
| IL2RB | 40 | interleukin 2 receptor, beta | SEQ ID | SEQ ID | SEQ ID |
| | | | No: 97 | No: 98 | No: 99 |
| EGFR | 57 | epidermal growth factor receptor (avian | SEQ ID | SEQ ID | SEQ ID |
| | | erythroblastic leukemia viral (v-erb-b) | No: 135 | No: 136 | No: 137 |
| | | oncogene homolog) | | | |
| IL2RG | 119 | interleukin 2 receptor, gamma (severe | SEQ ID | SEQ ID | SEQ ID |
| | | combined immunodeficiency) | No: 279 | No: 280 | No: 281 |
| CD3G | 174 | cd3g antigen, gamma polypeptide (tit3 | SEQ ID | SEQ ID | SEQ ID |
| | | complex) | No: 414 | No: 415 | No:416 |

Tables 9, 9A and 9B hereunder relate to subpopulations of polynucleotide sequences particularly interesting in classifying good and poor prognosis primary breast tumors.

TABLE 9

| Gene | SET | | | | |
|---|---|---|---|---|---|
| symbol | No | Name | Seq 3′ | Seq 5′ | Ref |
| CTSB | 14 | cathepsin b | | SEQ ID | SEQ ID |
| | | | | No: 30 | No: 31 |
| VIL2 | 23 | villin 2 (ezrin) | SEQ ID | SEQ ID | SEQ ID |
| | | | No: 51 | No: 52 | No: 53 |
| MUC1 | 25 | mucin 1, transmembrane | | SEQ ID | SEQ ID |
| | | | | No: 57 | No: 58 |
| EMR1 | 27 | egf-like module containing, mucin-like, | SEQ ID | SEQ ID | SEQ ID |
| | | hormone receptor-like sequence 1 | No: 62 | No: 63 | No: 64 |
| KIAA0427 | 28 | kiaa0427 gene product | SEQ ID | SEQ ID | SEQ ID |
| | | | No: 65 | No: 66 | No: 67 |
| GATA3 | 32 | gata-binding protein 3 | SEQ ID | SEQ ID | SEQ ID |
| | | | No: 76 | No: 77 | No: 78 |
| PRLR | 39 | prolactin receptor | SEQ ID | SEQ ID | SEQ ID |
| | | | No: 94 | No: 95 | No: 96 |
| GATA3 | 41 | gata-binding protein 3 | SEQ ID | SEQ ID | SEQ ID |
| | | | No: 100 | No: 101 | No: 78 |
| TC21 | 44 | oncogene tc21 | SEQ ID | SEQ ID | SEQ ID |
| | | | No: 106 | No: 107 | No: 108 |
| BCL2 | 48 | b-cell cll/lymphoma 2 | SEQ ID | SEQ ID | SEQ ID |
| | | | No: 115 | No: 116 | No: 117 |
| GATA3 | 51 | gata-binding protein 3 | SEQ ID | | SEQ ID |
| | | | No: 122 | | No: 78 |
| CRABP2 | 64 | cellular retinoic acid-binding protein 2 | SEQ ID | SEQ ID | SEQ ID |
| | | | No: 156 | No: 157 | No: 158 |
| ANG | 81 | angiogenin, ribonuclease, mase a | | SEQ ID | SEQ ID |
| | | family, 5 | | No: 194 | No: 195 |
| EGF | 83 | epidermal growth factor (beta- | SEQ ID | | SEQ ID |
| | | urogastrone) | No: 199 | | No: 200 |
| THBS1 | 91 | thrombospondin 1 | SEQ ID | | SEQ ID |
| | | | No: 216 | | No: 217 |
| EDNRA | 96 | endothelin receptor type a | SEQ ID | | SEQ ID |
| | | | No: 228 | | No: 229 |
| SMARCA2 | 99 | swi/snf related, matrix associated, actin | SEQ ID | SEQ ID | SEQ ID |
| | | dependent regulator of chromatin, | No: 235 | No: 236 | No: 237 |
| | | subfamily a, member 2 | | | |
| ABCB1 | 108 | atp-binding cassette, sub-family b | SEQ ID | | SEQ ID |
| | | (mdr/tap), member 1 | No: 257 | | No: 258 |
| EGF | 110 | epidermal growth factor (beta- | SEQ ID | | SEQ ID |
| | | urogastrone) | No: 262 | | No: 200 |
| BIRC4 | 116 | baculoviral iap repeat-containing 4 | SEQ ID | | SEQ ID |
| | | | No: 273 | | No: 274 |
| DAP3 | 117 | death associated protein 3 | SEQ ID | | SEQ ID |
| | | | No: 275 | | No: 276 |
| GNRH1 | 118 | gonadotropin-releasing hormone 1 | | SEQ ID | SEQ ID |
| | | (leutinizing-releasing hormone) | | No: 277 | No: 278 |
| DAP3 | 120 | death associated protein 3 | SEQ ID | SEQ ID | SEQ ID |
| | | | No: 282 | No: 283 | No: 276 |
| EST R97218 | 126 | ests, highly similar to tvhume | SEQ ID | SEQ ID | |
| | | hepatocyte growth factor receptor | No: 296 | No: 297 | |
| | | precursor [h. sapiens] | | | |
| BCL2 | 142 | b-cell cll/lymphoma 2 | SEQ ID | SEQ ID | SEQ ID |
| | | | No: 337 | No: 338 | No: 117 |
| BS69 | 144 | adenovirus 5 ela binding protein | SEQ ID | SEQ ID | SEQ ID |
| | | | No: 342 | No: 343 | No: 344 |
| MYB | 149 | v-myb avian myeloblastosis viral | | SEQ ID | SEQ ID |
| | | oncogene homolog | | No: 354 | No: 355 |
| CTSB | 152 | cathepsin b | SEQ ID | | SEQ ID |
| | | | No: 361 | | No: 31 |
| MLANA | 153 | melan-a | SEQ ID | SEQ ID | SEQ ID |
| | | | No: 362 | No: 363 | No: 364 |
| APR-1 | 154 | apr-1 protein | SEQ ID | SEQ ID | SEQ ID |
| | | | No: 365 | No: 366 | No: 367 |
| TC21 | 157 | oncogene tc21 | SEQ ID | SEQ ID | SEQ ID |
| | | | No: 372 | No: 373 | No: 108 |
| CDKN3 | 159 | cyclin-dependent kinase inhibitor 3 | SEQ ID | SEQ ID | SEQ ID |
| | | (cdk2-associated dual specificity | No: 377 | No: 378 | No: 379 |
| | | phosphatase) | | | |
| XBP1 | 162 | x-box binding protein 1 | SEQ ID | SEQ ID | SEQ ID |
| | | | No: 385 | No: 386 | No: 387 |
| CDH15 | 166 | cadherin 15, m-cadherin (myotubule) | SEQ ID | SEQ ID | SEQ ID |
| | | | No: 396 | No: 397 | No: 398 |
| BCL2 | 167 | b-cell cll/lymphoma 2 | SEQ ID | SEQ ID | SEQ ID |
| | | | No: 399 | No: 400 | No: 117 |
| EST W73386 | 168 | ests | SEQ ID | | |
| | | | No: 401 | | |
| ILF1 | 171 | interleukin enhancer binding factor 1 | SEQ ID | SEQ ID | SEQ ID |
| | | | No: 406 | No: 407 | No: 408 |
| ARHGDIA | 172 | rho gdp dissociation inhibitor (gdi) | SEQ ID | SEQ ID | SEQ ID |
| | | alpha | No: 409 | No: 410 | No: 411 |
| C4A | 173 | complement component 4a | SEQ ID | | SEQ ID |
| | | | No: 412 | | No: 413 |
| ESR1 | 176 | estrogen receptor 1 | SEQ ID | SEQ ID | SEQ ID |
| | | | No: 420 | No: 421 | No: 422 |
| PBX1 | 177 | pre-b-cell leukemia transcription factor | SEQ ID | SEQ ID | SEQ ID |
| | | 1 | No: 423 | No: 424 | No: 425 |
| GLI3 | 178 | gli-kruppel family member gli3 (greig | SEQ ID | SEQ ID | SEQ ID |
| | | cephalopolysyndactyly syndrome) | No: 426 | No: 427 | No: 428 |
| ILF1 | 179 | interleukin enhancer binding factor 1 | SEQ ID | | SEQ ID |
| | | | No: 429 | | No: 408 |
| ESTs | 184 | Homo sapiens aminoacylase 1 (ACY1). | SEQ ID | SEQ ID | |
| H24628 & | | | No: 435 | No: 436 | |
| H24592 | | | | | |
| EST H28056 | 185 | Homo sapiens E74-like factor 1 (ets | SEQ ID | | |
| | | domain transcription factor) (ELF1) | No: 437 | | |

TABLE 9A

| Gene | SET | | | | |
|---|---|---|---|---|---|
| symbol | No | Name | Seq 3′ | Seq 5′ | Ref |
| VIL2 | 23 | villin 2 (ezrin) | SEQ ID | SEQ ID | SEQ ID |
| | | | No: 51 | No: 52 | No: 53 |
| MUC1 | 25 | mucin 1, transmembrane | | SEQ ID | SEQ ID |
| | | | | No: 57 | No: 58 |
| GATA3 | 32 | gata-binding protein 3 | SEQ ID | SEQ ID | SEQ ID |
| | | | No: 76 | No: 77 | No: 78 |
| GATA3 | 41 | gata-binding protein 3 | SEQ ID | SEQ ID | SEQ ID |
| | | | No: 100 | No: 101 | No: 78 |
| BCL2 | 48 | b-cell cll/lymphoma 2 | SEQ ID | SEQ ID | SEQ ID |
| | | | No: 115 | No: 116 | No: 117 |
| GATA3 | 51 | gata-binding protein 3 | SEQ ID | | SEQ ID |
| | | | No: 122 | | No: 78 |
| CRABP2 | 64 | cellular retinoic acid-binding protein 2 | SEQ ID | SEQ ID | SEQ ID |
| | | | No: 156 | No: 157 | No: 158 |
| ANG | 81 | angiogenin, ribonuclease, rnase a family, 5 | | SEQ ID | SEQ ID |
| | | | | No: 194 | No: 195 |
| EGF | 83 | epidermal growth factor (beta-urogastrone) | SEQ ID | | SEQ ID |
| | | | No: 199 | | No: 200 |
| THBS1 | 91 | thrombospondin 1 | SEQ ID | | SEQ ID |
| | | | No: 216 | | No: 217 |
| SMARCA2 | 99 | swi/snf related, matrix associated, actin | SEQ ID | SEQ ID | SEQ ID |
| | | dependent regulator of chromatin, subfamily | No. 235 | No.236 | No: 237 |
| | | a, member 2 | | | |
| EGF | 110 | epidermal growth factor (beta-urogastrone) | SEQ ID | | SEQ ID |
| | | | No: 262 | | No: 200 |
| BIRC4 | 116 | baculoviral iap repeat-containing 4 | SEQ ID | | SEQ ID |
| | | | No: 273 | | No: 274 |
| BCL2 | 142 | b-cell cll/lymphoma 2 | SEQ ID | SEQ ID | SEQ ID |
| | | | No: 337 | No: 338 | No: 117 |
| BS69 | 144 | adenovirus 5 ela binding protein | SEQ ID | SEQ ID | SEQ ID |
| | | | No: 342 | No: 343 | No: 344 |
| MYB | 149 | v-myb avian myeloblastosis viral oncogene | | SEQ ID | SEQ ID |
| | | homolog | | No: 354 | No: 355 |
| XBP1 | 162 | x-box binding protein 1 | SEQ ID | SEQ ID | SEQ ID |
| | | | No: 385 | No: 386 | No: 387 |
| BCL2 | 167 | b-cell cll/lymphoma 2 | SEQ ID | SEQ ID | SEQ ID |
| | | | No: 399 | No: 400 | No: 117 |
| ILF1 | 171 | interleukin enhancer binding factor 1 | SEQ ID | SEQ ID | SEQ ID |
| | | | No: 406 | No: 407 | No: 408 |
| ARHGDIA | 172 | rho gdp dissociation inhibitor (gdi) alpha | SEQ ID | SEQ ID | SEQ ID |
| | | | No: 409 | No: 410 | No: 411 |
| C4A | 173 | complement component 4a | SEQ ID | | SEQ ID |
| | | | No: 412 | | No: 413 |
| ESR1 | 176 | estrogen receptor 1 | SEQ ID | SEQ ID | SEQ ID |
| | | | No: 420 | No: 421 | No: 422 |
| PBX1 | 177 | pre-b-cell leukemia transcription factor 1 | SEQ ID | SEQ ID | SEQ ID |
| | | | No: 423 | No: 424 | No: 425 |
| GLI3 | 178 | gli-kruppel family member gli3 (greig | SEQ ID | SEQ ID | SEQ ID |
| | | cephalopolysyndactyly syndrome) | No: 426 | No: 427 | No: 428 |
| ILF1 | 179 | interleukin enhancer binding factor 1 | SEQ ID | | SEQ ID |
| | | | No: 429 | | No: 408 |
| ESTs | 184 | Homo sapiens aminoacylase 1 (ACY1). | SEQ ID | SEQ ID | |
| H24628 & | | | No: 435 | No: 436 | |
| H24592 | | | | | |
| EST | 185 | Homo sapiens E74-like factor 1 (ets domain | SEQ ID | | |
| H28056 | | transcription factor) (ELF1) | No: 437 | | |

TABLE 9B

| Gene | | | | | |
|---|---|---|---|---|---|
| symbol | SET No | Name | Seq 3′ | Seq 5′ | Ref |
| CTSB | 14 | cathepsin b | | SEQ ID | SEQ ID |
| | | | | No: 30 | No: 31 |
| EMR1 | 27 | egf-like module containing, mucin-like, | SEQ ID | SEQ ID | SEQ ID |
| | | hormone receptor-like sequence 1 | No: 62 | No: 63 | No: 64 |
| KIAA0427 | 28 | kiaa0427 gene product | SEQ ID | SEQ ID | SEQ ID |
| | | | No: 65 | No: 66 | No: 67 |
| PRLR | 39 | prolactin receptor | SEQ ID | SEQ ID | SEQ ID |
| | | | No: 94 | No: 95 | No: 96 |
| TC21 | 44 | oncogene tc21 | SEQ ID | SEQ ID | SEQ ID |
| | | | No: 106 | No: 107 | No: 108 |
| EDNRA | 96 | endothelin receptor type a | SEQ ID | | SEQ ID |
| | | | No: 228 | | No: 229 |
| ABCB1 | 108 | atp-binding cassette, sub-family b | SEQ ID | | SEQ ID |
| | | (mdr/tap), member 1 | No: 257 | | No: 258 |
| DAP3 | 117 | death associated protein 3 | SEQ ID | | SEQ ID |
| | | | No: 275 | | No: 276 |
| GNRH1 | 118 | gonadotropin-releasing hormone 1 | | SEQ ID | SEQ ID |
| | | (leutinizing-releasing hormone) | | No: 277 | No: 278 |
| DAP3 | 120 | death associated protein 3 | SEQ ID | SEQ ID | SEQ ID |
| | | | No: 282 | No: 283 | No: 276 |
| EST R97218 | 126 | ests, highly similar to tvhume hepatocyte | SEQ ID | SEQ ID | |
| | | growth factor receptor precursor | No: 296 | No: 297 | |
| | | [h. sapiens] | | | |
| CTSB | 152 | cathepsin b | SEQ ID | | SEQ ID |
| | | | No: 361 | | No: 31 |
| MLANA | 153 | melan-a | SEQ ID | SEQ ID | SEQ ID |
| | | | No: 362 | No: 363 | No: 364 |
| APR-1 | 154 | apr-1 protein | SEQ ID | SEQ ID | SEQ ID |
| | | | No: 365 | No: 366 | No: 367 |
| TC21 | 157 | oncogene tc21 | SEQ ID | SEQ ID | SEQ ID |
| | | | No: 372 | No: 373 | No: 108 |
| CDKN3 | 159 | cyclin-dependent kinase inhibitor 3 (cdk2- | SEQ ID | SEQ ID | SEQ ID |
| | | associated dual specificity phosphatase) | No: 377 | No: 378 | No: 379 |
| CDH15 | 166 | cadherin 15, m-cadherin (myotubule) | SEQ ID | SEQ ID | SEQ ID |
| | | | No: 396 | No: 397 | No: 398 |
| EST | 168 | ests | SEQ ID | | |
| W73386 | | | No: 401 | | |

Overexpression of genes detected by using at least one polynucleotide sequence selected among those included in each one of predefined polynucleotide sequence sets indicated in table 9A combined with underexpression of genes detected with at least one polynucleotide sequence selected among those included in each one of predefined polynucleotide sequence sets indicated in table 9B present a Good outcome.

So, a preferred DNA array according to the invention comprises at least one polynucleotide sequence selected among those included in each one of predefined polynucleotide sequences indicated in table 9A and at least one polynucleotide sequence selected among those included in each one of predefined polynucleotide sequences indicated in table 9B.

Such DNA arrays are particularly useful to distinguish patients having a high risk (Bad Outcome) from those having a good prognosis (Good Outcome). References

1. DeRisi, J., Penland, L., Brown, P. O., Bittner, M. L., Meltzer, P. S., Ray, M., Chen, Y., Su, Y. A., and Trent, J. M. (1996) Use of a cDNA microarray to analyze gene expression patterns in human cancer. Nat Genet, 14, 457-460.

2. Jordan, B. R. (1998) Large-scale expression measurement by hybridization methods: from high- density membranes to “DNA chips”. J. Biochem (Tokyo), 124, 251-258.

3. Nguyen, C., Rocha, D., Granjeaud, S., Baldit, M., Bernard, K., Naquet, P., and Jordan, B. R. (1995) Differential gene expression in the murine thymus assayed by quantitative hybridization of arrayed cDNA clones. Genomics, 29, 207-216.

4. Bertucci, F., Van Hulst, S., Bernard, K., Loriod, B., Granjeaud, S., Tagett, R., Starkey, M., Nguyen, C., Jordan, B., and Birnbaum, D. (1999) Expression scanning of an array of growth control genes in human tumor cell lines. Oncogene, 18, 3905-3912.

5. Bertucci, F., Bernard, K., Loriod, B., Chang, Y. C., Granjeaud, S., Birnbaum, D., Nguyen, C., Peck, K., and Jordan, B. R. (1999) Sensitivity issues in DNA array-based expression measurements and performance of nylon microarrays for small samples [In Process Citation]. Hum Mol Genet, 8, 1715-1722.

6. Ross, J. S. and Fletcher, J. A. (1999) The HER-2/neu oncogene: prognostic factor, predictive factor and target for therapy. Semin Cancer Biol, 9, 125-138.

7. Scorilas, A., Trangas, T., Yotis, J., Pateras, C., and Talieri, M. (1999) Determination of c-myc amplification and overexpression in breast cancer patients: evaluation of its prognostic value against c-erbB-2, cathepsin-D and clinicopathological characteristics using univariate and multivariate analysis. Br J Cancer, 81, 1385-1391.

8. Fox, S. B., Smith, K., Hollyer, J., Greenall, M., Hastrich, D., and Harris, A. L. (1994) The epidermal growth factor receptor as a prognostic marker: results of 370 patients and review of 3009 patients. Breast Cancer Res Treat, 29, 41-49.

9. Heimann, R., Lan, F., McBride, R., and Hellman, S. (2000) Separating favorable from unfavorable prognostic markers in breast cancer: the role of E-cadherin. Cancer Res, 60, 298-304.

10. Guerin, M., Sheng, Z. M., Andrieu, N., and Riou, G. (1990) Strong association between c-myband oestrogen-receptor expression in human breast cancer. Oncogene , 5, 131-135.

11. Lim, K. C., Lakshmanan, G., Crawford, S. E., Gu, Y., Grosveld, F., and Douglas Engel, J. (2000) Gata 3 loss leads to embryonic lethality due to noradrenaline deficiency of the sympathetic nervous system. Nat Genet, 25, 209-212.

12. Mills, K. J., Vollberg, T. M., Nervi, C., Grippo, J. F., Dawson, M. I., and Jetten, A. M. (1996) Regulation of retinoid-induced differentiation in embryonal carcinoma PCC 4.azalR cells: effects of retinoid-receptor selective ligands. Cell Growth Differ, 7, 327-337.

13. Easty, D. J., Hill, S. P., Hsu, M. Y., Fallowfield, M. E., Florenes, V. A., Herlyn, M., and Bennett, D. C. (1999) Up-regulation of ephrin0A1 during melanoma progression. Int J Cancer, 84, 494-501.

14. Shim, C., Zhang, W., Rhee, C. H., and Lee, J. H. (1998) Profiling of differentially expressed genes in human primary cervical cancer by complementary DNA expression array. Clin Cancer Res, 4, 3045-3050.

15. Tsou, A. P., Wu, K. M., Tsen, T. Y., Chi, C. W., Chiu, J. H., Lui, W. Y., Hu, C. P., Chang, C., Chou, C. K., and Tsai, S. F. (1998) Parallel hybridization analysis of multiple protein kinase genes: identification of gene expression patterns characteristic of human hepatocellular carcinoma. Genomics, 50, 331-340.

16. Schummer, M., Ng, W. V., Bumgarner, R. E., Nelson, P. S., Schummer, B., Bednarski, D. W., Hassell, L., Baldwin, R. L., Karlan, B. Y., and Hood, L. (1999) Comparative hybridization of an array of 21,500 ovarian cDNAs for the discovery of genes overexpressed in ovarian carcinomas. Gene, 238, 375-385.

17. Alon, U., Barkai, N., Notterman, D. A., Gish, K., Ybarra, S., Mack, D., and Levine, A. J. (1999) Broad patterns of gene expression revealed by clustering analysis of tumor and normal colon tissues probed by oligonucleotide arrays. Proc Natl Acad Sci U S A, 96, 6745-6750.

18. Moch, H., Schraml, P., Bubendorf, L., Mirlacher, M., Kononen, J., Gasser, T., Mihatsch, M. J., Kallioniemi, O. P., and Sauter, G. (1999) High-throughput tissue microarray analysis to evaluate genes uncovered by cDNA microarray screening in renal cell carcinoma. Am J Pathol, 154, 981-986.

19. Rhee, C. H., Hess, K., Jabbur, J., Ruiz, M., Yang, Y., Chen, S., Chenchik, A., Fuller, G. N., and Zhang, W. (1999) cDNA expression array reveals heterogeneous gene expression profiles in three glioblastoma cell lines. Oncogene, 18, 2711-2717.

20. Huang, F., Adelman, J., Jiang, H., Goldstein, N. I., and Fisher, P. B. (1999) Identification and temporal expression pattern of genes modulated during irreversible growth arrest and terminal differentiation in human melanoma cells. Oncogene, 18, 3546-3552.

21. Bittner, M., Meltzer, P., Chen, Y., Jiang, Y., Seftor, E., Hendrix, M., Radmacher, M., Simon, R., Yakhini, Z., Ben-Dor, A., Sampas, N., Dougherty, E., Wang, E., Marincola, F., Gooden, C., Lueders, J., Glatfelter, A., Pollock, P., Carpten, J., Gillanders, E., Leja, D., Dietrich, K., Beaudry, C., Berens, M., Alberts, D., and Sondak, V. (2000) Molecular classification of cutaneous malignant melanoma by gene expression profiling. Nature, 406, 536-540.

22. Khan, J., Simon, R., Bittner, M., Chen, Y., Leighton, S. B., Pohida, T., Smith, P. D., Jiang, Y., Gooden, G. C., Trent, J. M., and Meltzer, P. S. (1998) Gene expression profiling of alveolar rhabdomyosarcoma with cDNA microarrays. Cancer Res, 58, 5009-5013.

23. Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D., and Lander, E. S. (1999) Molecular classification of cancer: class discovery and class prediction by gene expression monitoring. Science, 286, 531-537.

24. Alizadeh, A. A., Eisen, M. B., Davis, R. E., Ma, C., Lossos, I. S., Rosenwald, A., Boldrick, J. C., Sabet, H., Tran, T., Yu, X., Powell, J. I., Yang, L., Marti, G. E., Moore, T., Hudson, J., Jr., Lu, L., Lewis, D. B., Tibshirani, R., Sherlock, G., Chan, W. C., Greiner, T. C., Weisenburger, D. D., Armitage, J. O., Warnke, R., and Staudt, L. M. (2000) Distinct types of diffuse large B-cell lymphoma identified by gene expression profiling [In Process Citation]. Nature, 403, 503-511.

25. Hoch, R. V., Thompson, D. A., Baker, R. J., and Weigel, R. J. (1999) GATA-3 is expressed in association with estrogen receptor in breast cancer. Int J Cancer, 84, 122-128.

26. Hilsenbeck, S. G., Friedrichs, W. E., Schiff, R., O° Connell, P., Hansen, R. K., Osborne, C. K., and Fuqua, S. A. (1999) Statistical analysis of array expression data as applied to the problem of tamoxifen resistance. J Natl Cancer Inst, 91, 453-459.

27. Martin, K. J., Kritzman, B. M., Price, L. M., Koh, B., Kwan, C. P., Zhang, X., Mackay, A., O'Hare, M. J., Kaelin, C. M., Mutter, G. L., Pardee, A. B., and Sager, R. (2000) Linking gene expression patterns to therapeutic groups in breast cancer. Cancer Res, 60, 2232-2238.

28. Yang, G. P., Ross, D. T., Kuang, W. W., Brown, P. O., and Weigel, R. J. (1999) Combining SSH and cDNA microarrays for rapid identification of differentially expressed genes. Nucleic Acids Res, 27, 1517-1523.

29. Perou, C. M., Jeffrey, S. S., van de Rijn, M., Rees, C. A., Eisen, M. B., Ross, D. T., Pergamenschikov, A., Williams, C. F., Zhu, S. X., Lee, J. C., Lashkari, D., Shalon, D., Brown, P. O., and Botstein, D. (1999) Distinctive gene expression patterns in human mammary epithelial cells and breast cancers. Proc Natl Acad Sci U S A, 96, 9212-9217.

30. Nacht, M., Ferguson, A. T., Zhang, W., Petroziello, J. M., Cook, B. P., Gao, Y. H., Maguire, S., Riley, D., Coppola, G., Landes, G. M., Madden, S. L., and Sukumar, S. (1999) Combining serial analysis of gene expression and array technologies to identify genes differentially expressed in breast cancer. Cancer Res, 59, 5464-5470.

31. Sgroi, D. C., Teng, S., Robinson, G., LeVangie, R., Hudson, J. R., Jr., and Elkahloun, A. G. (1999) In vivo gene expression profile analysis of human breast cancer progression. Cancer Res, 59, 5656-5661.

32. Perou, C. M., Sorlie, T., Eisen, M. B., van de Rijn, M., Jeffrey, S. S., Rees, C. A., Pollack, J. R., Ross, D. T., Johnsen, H., Akslen, L. A., Fluge, O., Pergamenschikov, A., Williams, C., Zhu, S. X., Lonning, P. E., Borresen-Dale, A. L., Brown, P. O., and Botstein, D. (2000) Molecular portraits of human breast tumours. Nature, 406, 747-752.

33. Hahnel, E., Harvey, J. M., Joyce, R., Robbins, P. D., Sterrett, G. F., and Hahnel, R. (1993) Stromelysin-3 expression in breast cancer biopsies: clinico- pathological correlations. Int J Cancer, 55, 771-774.

34. Skoog, L., Humla, S., Klintenberg, C., Pasqual, M., and Wallgren, A. (1985) Receptors for retinoic acid and retinol in human mammary carcinomas. Eur J Cancer Clin Oncol, 21, 901-906.

35. Thor, A. D., Moore, D. H., I I, Edgerton, S. M., Kawasaki, E. S., Reihsaus, E., Lynch, H. T., Marcus, J. N., Schwartz, L., Chen, L. C., Mayall, B. H., and et al. (1992) Accumulation of p 53 tumor suppressor gene protein: an independent marker of prognosis in breast cancers. J Natl Cancer Inst, 84, 845-855.

36. Allred, D. C., Harvey, J. M., Berardo, M., and Clark, G. M. (1998) Prognostic and predictive factors in breast cancer by immunohistochemical analysis. Mod Pathol , 11, 155-168.

37. Spencer, K. S., Graus-Porta, D., Leng, J., Hynes, N. E., and Klemke, R. L. (2000) ErbB 2 is necessary for induction of carcinoma cell invasion by ErbB family receptor tyrosine kinases. J Cell Biol, 148, 385-397.

38. Behrens, J. (1993) The role of cell adhesion molecules in cancer invasion and metastasis. Breast Cancer Res Treat, 24, 175-184.

39. Roberts, D. D. (1996) Regulation of tumor growth and metastasis by thrombospondin-1. Faseb J, 10, 1183-1191.

40. Taylor-Papadimitriou, J., Burchell, J., Miles, D. W., and Dalziel, M. (1999) MUCI and cancer. Biochim Biophys Acta, 1455, 301-313.

41. Sneath, R. J. and Mangham, D. C. (1998) The normal structure and function of CD44 and its role in neoplasia. Mol Pathol, 51, 191-200.

42. Iyer, V. R., Eisen, M. B., Ross, D. T., Schuler, G., Moore, T., Lee, J. C. F., Trent, J. M., Staudt, L. M., Hudson, J., Jr., Boguski, M. S., Lashkari, D., Shalon, D., Botstein, D., and Brown, P. O. (1999) The transcriptional program in the response of human fibroblasts to serum. Science, 283, 83-87.

43. Theillet, C., Adelaide, J., Louason, G., Bonnet-Dorion, F., Jacquemier, J., Adnane, J., Longy, M., Katsaros, D., Sismondi, P., Gaudray, P., and et al. (1993) FGFRI and PLAT genes and DNA amplification at 8p12 in breast and ovarian cancers. Genes Chromosomes Cancer, 7, 219-226.

44. Granjeaud, S., Nguyen, C., Rocha, D., Luton, R., and Jordan, B. R. (1996) From hybridization image to numerical values: a practical, high throughput quantification system for high density filter hybridizations. Genet Anal, 12, 151-162.

45. Eisen, M. B., Spellman, P. T., Brown, P. O., and Botstein, D. (1998) Cluster analysis and display of genome-wide expression patterns. Proc Natl Acad Sci U S A , 95, 14863-14868.

46. Ferrari, S., Battini, R., and Cossu, G. (1990) Differentiation-dependent expression of apolipoprotein A-I in chicken myogenic cells in culture. Dev Biol, 140, 430-436.

1. A polynucleotide library useful in the molecular characterization of a carcinoma, said library comprising a pool of polynucleotide sequences or subsequences thereof wherein said sequences or subsequences are either underexpressed or overexpressed in tumor cells, further wherein said sequences or subsequences correspond substantially to any of the polynucleotide sequences set forth in any of SEQ ID Nos: 1-468 or the complement thereof.

2. A polynucleotide library useful in the molecular characterization of a carcinoma, said library comprising a pool of polynucleotide sequences or subsequences thereof wherein said sequences or subsequences are overexpressed or underexpressed in tumor cells, further wherein said sequences or subsequences correspond substantially to any of the polynucleotide sequences set forth in any of SEQ ID No: 1, SEQ ID No: 3, SEQ ID No: 5, SEQ ID No: 7, SEQ ID No: 8, SEQ ID No: 9, SEQ ID No: 11, SEQ ID No: 13, SEQ ID No: 14, SEQ ID No: 16, SEQ ID No: 17, SEQ ID No: 18, SEQ ID No: 20, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60, SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 91, SEQ ID No: 94, SEQ ID No: 95, SEQ ID No: 97, SEQ ID No: 98, SEQ ID No: 102, SEQ ID No: 104, SEQ ID No: 109, SEQ ID No: 111, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 138, SEQ ID No: 139, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 168, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 189, SEQ ID No: 190, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 280, SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 335, SEQ ID No: 339, SEQ ID No: 340, SEQ ID No: 345, SEQ ID No: 347, SEQ ID No: 350, SEQ ID No: 352, SEQ ID No: 354, SEQ ID No: 356, SEQ ID No: 358, SEQ ID No: 359, SEQ ID No: 368, SEQ ID No: 369, SEQ ID No: 370, SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 404, SEQ ID No: 405, SEQ ID No: 414, SEQ ID No: 415, SEQ ID No: 417, SEQ ID No: 418, SEQ ID No: 430, SEQ ID No: 431, SEQ ID No: 435, SEQ ID No: 437, SEQ ID No: 439, SEQ ID No: 440, SEQ ID No: 443, SEQ ID No: 444, SEQ ID No: 445, SEQ ID No: 446, SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, SEQ ID No: 458, SEQ ID No: 459, SEQ ID No: 460, SEQ ID No: 461, SEQ ID No: 462, SEQ ID No: 463, SEQ ID No: 464, SEQ ID No: 465, SEQ ID No: 466, SEQ ID No: 467, SEQ ID No: 468 or the complement thereof.

3. The polynucleotide library of claim 2 wherein said tumor cells are breast tumor cells.

4. The polynucleotide library of claim 2 wherein the pool of polynucleotide sequences or subsequences correspond substantially to the polynucleotide sequences set forth in any of SEQ ID No: 1, SEQ ID No: 3, SEQ ID No: 5, SEQ ID No: 7, SEQ ID No: 8, SEQ ID No: 9, SEQ ID No: 11, SEQ ID No: 13, SEQ ID No: 14 SEQ ID No: 16, SEQ ID No: 17, SEQ ID No: 18, SEQ ID No: 20, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60, SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 91, SEQ ID No: 94, SEQ ID No: 95, SEQ ID No: 97, SEQ ID No: 98, SEQ ID No: 102, SEQ ID No: 104, SEQ ID No: 109, SEQ ID No: 111, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 138, SEQ ID No: 139, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 168, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 280, SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 335, SEQ ID No: 339, SEQ ID No: 340, SEQ ID No: 345, SEQ ID No: 347, SEQ ID No: 350, SEQ ID No: 352, SEQ ID No: 354, SEQ ID No: 356, SEQ ID No: 358, SEQ ID No: 359, SEQ ID No: 368, SEQ ID No: 369, SEQ ID No: 370, SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 404, SEQ ID No: 405, SEQ ID No: 414, SEQ ID No: 415, SEQ ID No: 417, SEQ ID No: 418, SEQ ID No: 430, SEQ ID No: 431, SEQ ID No: 435, SEQ ID No: 437, SEQ ID No: 439, SEQ ID No: 440, SEQ ID No: 443, SEQ ID No: 444, SEQ ID No: 445, SEQ ID No: 446, SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, SEQ ID No: 458, SEQ ID No: 459, SEQ ID No: 460, SEQ ID No: 461, SEQ ID No: 462, SEQ ID No: 463, SEQ ID No: 464, SEQ ID No: 465, SEQ ID No: 466, SEQ ID No: 467, SEQ ID No: 468; and
further wherein said polynucleotide sequences or subsequences of said pool are useful in differentiating a normal cell from a cancer cell.

5. The polynucleotide library of claim 2 wherein the pool of polynucleotide sequences or subsequences correspond substantially to the polynucleotide sequences set forth in any of SEQ ID No: 1, SEQ ID No: 3, SEQ ID No: 5, SEQ ID No: 7, SEQ ID No: 8, SEQ ID No: 9, SEQ ID No: 11, SEQ ID No: 13, SEQ ID No: 14, SEQ ID No: 16, SEQ ID No: 17, SEQ ID No: 18, SEQ ID No: 20, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60 , SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 94, SEQ ID No: 95, SEQ ID No: 97, SEQ ID No: 98, SEQ ID No: 102, SEQ ID No: 104, SEQ ID No: 109, SEQ ID No: I-l, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 280, SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 335, SEQ ID No: 339, SEQ ID No: 340, SEQ ID No: 345, SEQ ID No: 347, SEQ ID No: 350, SEQ ID No: 352, SEQ ID No: 354, SEQ ID No: 356, SEQ ID No: 358, SEQ ID No: 359, SEQ ID No: 368, SEQ ID No: 369, SEQ ID No: 370, SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 404, SEQ ID No: 405, SEQ ID No: 414, SEQ ID No: 415, SEQ ID No: 417, SEQ ID No: 418, SEQ ID No: 430, SEQ ID No: 431, SEQ ID No: 435, SEQ ID No: 437, SEQ ID No: 439, SEQ ID No: 440, SEQ ID No: 443, SEQ ID No: 444, SEQ ID No: 445, SEQ ID No: 446, SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, SEQ ID No: 458, SEQ ID No: 459, SEQ ID No: 460, SEQ ID No: 461, SEQ ID No: 462, SEQ ID No: 463, SEQ ID No: 464, SEQ ID No: 465, SEQ ID No: 466, SEQ ID No: 467, and
wherein said polynucleotide sequences or subsequences of said pool are useful in detecting a hornone-sensitive tumor cell.

6. The library polynucleotide of claim 2 wherein the pool of polynucleotide sequences or subsequences correspond substantially to the polynucleotide sequences set forth in any of SEQ ID No: 1, SEQ ID No: 3, SEQ ID No: 5, SEQ ID No: 7, SEQ ID No: 8, SEQ ID No: 9, SEQ ID No: 1, SEQ ID No: 13, SEQ ID No: 14, SEQ ID No: 16, SEQ ID No: 17, SEQ ID No: 18, SEQ ID No: 20, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60, SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 94, SEQ ID No: 95, SEQ ID No: 97, SEQ ID No: 98, SEQ ID No: 102, SEQ ID No: 104, SEQ ID No: 109, SEQ ID No: 111, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 280, SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 335, SEQ ID No: 339, SEQ ID No: 340, SEQ ID No: 345, SEQ ID No: 347, SEQ ID No: 350, SEQ ID No: 352, SEQ ID No: 354, SEQ ID No: 356, SEQ ID No: 358, SEQ ID No: 359, SEQ ID No: 368, SEQ ID No: 369, SEQ ID No: 370, SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 404, SEQ ID No: 405, SEQ ID No: 414, SEQ ID No: 415, SEQ ID No: 417, SEQ ID No: 418, SEQ ID No: 430, SEQ ID No: 431, SEQ ID No: 435, SEQ ID No: 437, SEQ ID No: 439, SEQ ID No: 440, SEQ ID No: 443, SEQ ID No: 444, SEQ ID No: 445, SEQ ID No: 446, SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, SEQ ID No: 458, SEQ ID No: 459, SEQ ID No: 460, SEQ ID No: 461, SEQ ID No: 462, SEQ ID No: 463, SEQ ID No: 464, SEQ ID No: 465, SEQ ID No: 466, SEQ ID No: 467; and
wherein said polynucleotide sequences or subsequences of said pool are useful in differentiating a tumor in which a lymph node has been invaded by a tumor cell from a tumor in which a lymph node has not been invaded by a tumor cell.

7. The polynucleotide library of claim 2 wherein the pool of polynucleotide sequences or subsequences correspond substantially to the polynucleotide sequences set forth in any of SEQ ID No: 1, SEQ ID No: 3, SEQ ID No: 5, SEQ ID No: 7, SEQ ID No: 8, SEQ ID No: 9, SEQ ID No: 11, SEQ ID No: 13, SEQ ID No: 14, SEQ ID No: 16, SEQ ID No: 17, SEQ ID No: 18, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60, SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 94, SEQ ID No: 95, SEQ ID No: 102, SEQ ID No: 104, SEQ ID No: 109, SEQ ID No: 111, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 280, SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 332, SEQ ID No: 339, SEQ ID No: 340, SEQ ID No: 345, SEQ ID No: 347, SEQ ID No: 350, SEQ ID No: 332, SEQ ID No: 354, SEQ ID No: 356, SEQ ID No: 358, SEQ ID No: 359, SEQ ID No: 368, SEQ ID No: 369, SEQ ID No: 370, SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 404, SEQ ID No: 405, SEQ ID No: 414, SEQ ID No: 415, SEQ ID No: 417, SEQ ID No: 418, SEQ ID No: 439, SEQ ID No: 443, SEQ ID No: 444, SEQ ID No: 445, SEQ ID No: 446, SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, and
wherein said polynucleotide sequences or subsequences of said pool are usefull in differentiating anthracycline-sensitive tumors from anthracycline-insensitive tumors.

8. The polynucleotide library of any of claims 2-7 wherein said polynucleotide sequences or subsequences of said pool are immobilized on a solid support in order to form a polynucleotide array.

9. The polynucleotide library of claim 8 wherein the solid support is selected from the group consisting of a nylon membrane, glass slide, glass beads, or a silicon chip.

10. The polynucleotide library of claim 8 wherein the solid support is a membrane on a glass support.

11. A method for detecting differentially expressed polynucleotide sequences which are correlated with a cancer, said method comprising:
obtaining a polynucleotide sample from a patient;
labeling said polynucleotide sample by reacting said polynucleotide sample with a labeled probe immobilized on a solid support wherein said probe comprises any of the polynucleotide sequences of the polynucleotide library of claim 2 or an expression product encoded by any of the polynucleotide sequences of the polynucleotide library of claim 2; and
detecting a polynucleotide sample reaction product.

12. The method of claim 11 further comprising obtaining a control polynucleotide sample, labeling said control sample by reacting said control sample with said labeled probe, detecting a control sample reaction product, and comparing the amount of said polynucleotide sample reaction product to the amount of said control sample reaction product.

13. The method of claims 11 or 12 wherein RNA or mRNA is isolated from said polynucleotide sample.

14. The method of claim 13 wherein mRNA is isolated from said polynucleotide sample and cDNA is obtained by reverse transcription of said niRNA.

15. The method of claim 11 wherein said labeling is performed by hybridizing the polynucleotide sample with the labeled probe.

16. The method of claim 13 wherein said method is used for detecting, diagnosing, staging, monitoring, predicting, preventing or treating conditions associated with cancer.

17. The method of claim 11 wherein the cancer is breast cancer.

18. The method of claim 11 wherein the product encoded by any of the polynucleotide sequences or subsequences is involved in a receptor-ligand reaction on which detection is based.

19. A method for screening an anti-tumor agent comprising the method of claim 11 wherein the polynucleotide sample has been treated with an anti-tumor agent to be screened.

20. The method of claim 11 wherein the label is selected from the group consisting of radioactive, colorimetric, enzymatic, molecular amplification, bioluminescent or fluorescent labels.

21. A library of polynucleotides comprising a population of polynucleotide sequences overexpressed or underexpressed in cells derived from a tumor selected from SEQ ID No: 1, SEQ ID No: 3, SEQ ID No: 5, SEQ ID No: 7, SEQ ID No: 8, SEQ ID No: 9, SEQ ID No: 11, SEQ ID No: 13, SEQ ID No: 14, SEQ ID No: 16, SEQ ID No: 17, SEQ ID No: 18, SEQ ID No: 20, SEQ ID No: 22, SEQ ID No: 23, SEQ ID No: 25, SEQ ID No: 27, SEQ ID No: 28, SEQ ID No: 30, SEQ ID No: 32, SEQ ID No: 33, SEQ ID No: 35, SEQ ID No: 36, SEQ ID No: 37, SEQ ID No: 39, SEQ ID No: 40, SEQ ID No: 42, SEQ ID No: 44, SEQ ID No: 46, SEQ ID No: 48, SEQ ID No: 49, SEQ ID No: 51, SEQ ID No: 52, SEQ ID No: 54, SEQ ID No: 55, SEQ ID No: 57, SEQ ID No: 59, SEQ ID No: 60, SEQ ID No: 65, SEQ ID No: 66, SEQ ID No: 68, SEQ ID No: 69, SEQ ID No: 71, SEQ ID No: 73, SEQ ID No: 74, SEQ ID No: 76, SEQ ID No: 79, SEQ ID No: 80, SEQ ID No: 82, SEQ ID No: 86, SEQ ID No: 88, SEQ ID No: 89, SEQ ID No: 91, SEQ ID No: 94, SEQ ID No: 95, SEQ ID No: 97, SEQ ID No: 98, SEQ ID No: 102, SEQ ID No: 104, SEQ ID No: 109, SEQ ID No: 111, SEQ ID No: 112, SEQ ID No: 114, SEQ ID No: 115, SEQ ID No: 116, SEQ ID No: 120, SEQ ID No: 123, SEQ ID No: 126, SEQ ID No: 127, SEQ ID No: 129, SEQ ID No: 131, SEQ ID No: 133, SEQ ID No: 135, SEQ ID No: 136, SEQ ID No: 138, SEQ ID No: 139, SEQ ID No: 141, SEQ ID No: 142, SEQ ID No: 144, SEQ ID No: 145, SEQ ID No: 147, SEQ ID No: 148, SEQ ID No: 150, SEQ ID No: 153, SEQ ID No: 154, SEQ ID No: 156, SEQ ID No: 157, SEQ ID No: 159, SEQ ID No: 160, SEQ ID No: 162, SEQ ID No: 164, SEQ ID No: 166, SEQ ID No: 167, SEQ ID No: 168, SEQ ID No: 171, SEQ ID No: 173, SEQ ID No: 174, SEQ ID No: 176, SEQ ID No: 178, SEQ ID No: 180, SEQ ID No: 181, SEQ ID No: 183, SEQ ID No: 185, SEQ ID No: 186, SEQ ID No: 187, SEQ ID No: 189, SEQ ID No: 190, SEQ ID No: 192, SEQ ID No: 194, SEQ ID No: 196, SEQ ID No: 197, SEQ ID No: 201, SEQ ID No: 202, SEQ ID No: 204, SEQ ID No: 205, SEQ ID No: 206, SEQ ID No: 208, SEQ ID No: 212, SEQ ID No: 214, SEQ ID No: 216, SEQ ID No: 218, SEQ ID No: 219, SEQ ID No: 221, SEQ ID No: 223, SEQ ID No: 224, SEQ ID No: 226, SEQ ID No: 230, SEQ ID No: 231, SEQ ID No: 233, SEQ ID No: 238, SEQ ID No: 239, SEQ ID No: 240, SEQ ID No: 242, SEQ ID No: 243, SEQ ID No: 245, SEQ ID No: 246, SEQ ID No: 248, SEQ ID No: 250, SEQ ID No: 251, SEQ ID No: 253, SEQ ID No: 255, SEQ ID No: 259, SEQ ID No: 260, SEQ ID No: 263, SEQ ID No: 265, SEQ ID No: 269, SEQ ID No: 271, SEQ ID No: 277, SEQ ID No: 279, SEQ ID No: 2 , SEQ ID No: 284, SEQ ID No: 286, SEQ ID No: 287, SEQ ID No: 289, SEQ ID No: 291, SEQ ID No: 293, SEQ ID No: 294, SEQ ID No: 296, SEQ ID No: 298, SEQ ID No: 299, SEQ ID No: 301, SEQ ID No: 302, SEQ ID No: 303, SEQ ID No: 305, SEQ ID No: 306, SEQ ID No: 308, SEQ ID No: 309, SEQ ID No: 311, SEQ ID No: 312, SEQ ID No: 314, SEQ ID No: 315, SEQ ID No: 317, SEQ ID No: 319, SEQ ID No: 320, SEQ ID No: 322, SEQ ID No: 323, SEQ ID No: 324, SEQ ID No: 326, SEQ ID No: 327, SEQ ID No: 329, SEQ ID No: 331, SEQ ID No: 332, SEQ ID No: 334, SEQ ID No: 335, SEQ ID No: 339, SEQ ID No: 340, SEQ ID No: 345, SEQ ID No: 347, SEQ ID No: 350, SEQ ID No: 352, SEQ ID No: 354, SEQ ID No: 356, SEQ ID No: 358, SEQ ID No: 359, SEQ ID No: 368, SEQ ID No: 369, SEQ ID No: 3 , SEQ ID No: 374, SEQ ID No: 375, SEQ ID No: 380, SEQ ID No: 382, SEQ ID No: 383, SEQ ID No: 385, SEQ ID No: 386, SEQ ID No: 388, SEQ ID No: 392, SEQ ID No: 394, SEQ ID No: 402, SEQ ID No: 401, SEQ ID No: 430, SEQ ID No: 414, SEQ ID No: 415, SEQ ID No: 417, SEQ ID No: 418, SEQ ID No: 430, SEQ ID No: 431, SEQ ID No: 435, SEQ ID No: 437, SEQ ID No: 439, SEQ ID No: 440, SEQ ID No: 443, SEQ ID No: 444, SEQ ID No: 45 , SEQ ID No: 4 , SEQ ID No: 447, SEQ ID No: 448, SEQ ID No: 449, SEQ ID No: 450, SEQ ID No: 451, SEQ ID No: 452, SEQ ID No: 453, SEQ ID No: 454, SEQ ID No: 455, SEQ ID No: 456, SEQ ID No: 457, SEQ ID No: 45, SEQ ID No: 459, SEQ ID No: 460, SEQ ID No: 461, SEQ ID No: 462, SEQ ID No: 463, SEQ ID No: 464, SEQ ID No: 465, SEQ ID No: 466, SEQ ID No: 467, SEQ ID No: 468 and their respective complements.

22. A library of polynucleotide sequences for distinguishing a person with cancer from a person without cancer comprising SEQ ID No: 1, SEQ ID No: 7, SEQ ID No: 8, SEQ ID No: 39, SEQ ID No: 40, SEQ ID No: 46, SEQ ID No: 54, SEQ ID No: 55, SEQ ID No: 76, SEQ ID No: 91, SEQ ID No: 115, SEQ ID No: 116, SEQ ID No: 126, SEQ ID No: 127, SEQ ID No: 138, SEQ ID No: 139, SEQ ID No: 141, SEQ ID No: 142, SEQ ID No: 147, SEQ ID No: 148, SEQ ID No: 156, SEQ ID No: 157, SEQ ID No: 162, SEQ ID No: 168, SEQ ID No: 178, SEQ ID No: 204, SEQ ID No: 205, SEQ ID No: 216, SEQ ID No: 230, SEQ ID No: 231, SEQ ID No: 248, SEQ ID No: 250, SEQ ID No: 251, SEQ ID No: 265, SEQ ID No: 271, SEQ ID No: 308, SEQ ID No: 309, SEQ ID No: 311, SEQ ID No: 312, SEQ ID No: 317, SEQ ID No: 324, SEQ ID No: 345, SEQ ID No: 350, SEQ ID No: 368, SEQ ID No: 369, SEQ ID No: 417, SEQ ID No: 418, SEQ ID No: 430, SEQ ID No: 431, SEQ ID No: 437, SEQ ID No: 440, SEQ ID No: 450, SEQ ID No: 452, SEQ ID No: 453, SEQ ID No: 454, SEQ ID No: 460andSEQ ID No: 468.

23. A library of polynucleotide sequences for detecting hormone-sensitive tumors comprising SEQ ID No: 22, SEQ ID No: 23, SEQ ID No: 59, SEQ ID No: 60, SEQ ID No: 76, SEQ ID No: 82, SEQ ID No: 97, SEQ ID No: 98, SEQ ID No: 135, SEQ ID No: 136, SEQ ID No: 156, SEQ ID No: 157, SEQ ID No: 255, SEQ ID No: 279, SEQ ID No: 280, SEQ ID No: 322, SEQ ID No: 323, SEQ ID No: 331, SEQ ID No: 332, SEQ ID No: 334, SEQ ID No: 335, SEQ ID No: 345, SEQ ID No: 352, SEQ ID No: 354, SEQ ID No: 385, SEQ ID No: 386, SEQ ID No: 394, SEQ ID No: 402, SEQ ID No: 414, SEQ ID No: 415, SEQ ID No: 443 and SEQ ID No: 457.

24. A library of polynucleotide sequences for distinguishing a tumor in which a lymph node has been invaded by a tumor cell from a tumor in which a lymph node has not been invaded by a tumor cell comprising SEQ ID No: 16, SEQ ID No: 22, SEQ ID No: 23, SEQ ID No: 39, SEQ ID No: 40, SEQ ID No: 57, SEQ ID No: 76, SEQ ID No: 82, SEQ ID No: 97, SEQ ID No: 98, SEQ ID No: 135, SEQ ID No: 136, SEQ ID No: 216, SEQ ID No: 238, SEQ ID No: 239, SEQ ID No: 250, SEQ ID No: 251, SEQ ID No: 322, SEQ ID No: 323, SEQ ID No: 326, SEQ ID No: 327, SEQ ID No: 329, SEQ ID No: 334, SEQ ID No: 335, SEQ ID No: 374, SEQ ID No: 375, SEQ ID No: 402, SEQ ID No: 430, SEQ ID No: 439, SEQ ID No: 444, SEQ ID No: 445 and SEQ ID No: 457.

25. A library of polynucleotide sequences for distinguishing anthracycline-sensitive tumors from anthracyline-insensitive tumors comprising SEQ ID No: 22, SEQ ID No: 23, SEQ ID No: 48, SEQ ID No: 49, SEQ ID No: 51, SEQ ID No: 52, SEQ ID No: 59, SEQ ID No: 60, SEQ ID No: 65, SEQ ID No: 66, SEQ ID No: 73, SEQ ID No: 74, SEQ ID No: 76, SEQ ID No: 82, SEQ ID No: 86, SEQ ID No: 135, SEQ ID No: 136, SEQ ID No: 156, SEQ ID No: 157, SEQ ID No: 178, SEQ ID No: 194, SEQ ID No: 226, SEQ ID No: 308, SEQ ID No: 309, SEQ ID No: 326, SEQ ID No: 327, SEQ ID No: 331, SEQ ID No: 332, SEQ ID No: 354, SEQ ID No: 385, SEQ ID No: 386, SEQ ID No: 392, SEQ ID No: 394, SEQ ID No: 433, SEQ ID No-. 434, SEQ ID No: 444 and SEQ ID No: 456.

26. A method of detecting differentially expressed genes correlated with a cancer comprising detecting at least one polynucleotide sequence or subsequence of a polynucleotide library according to claim 2 or detecting at least one product encoded by said polynucleotide library in a sample obtained from a patient.

27. A method according to claim 26 further comprising comparing an amount of said at least one polynucleotide sequence or subsequence or product encoded by said polynucleotide sequence with an amount of said polynucleotide sequence or subsequence or product encoded by said polynucleotide sequence or subsequence obtained from a control sample.

28. A method according to claim 26 comprising extracting mRNA from said polynucleotide sample.

29. A method according to claim 28 comprising reverse transcribing said mRNA to cDNA.

30. The method according to claim 26 comprising hybridizing said at least one polynucleotide sequence or subsequence with mRNA or cDNA from the polynucleotide sample.

31. The method of claim 26 comprising detecting, diagnosing, staging, monitoring, predicting, preventing or treating conditions associated with cancer.

32. The method according to claim 26 wherein the product encoded by any of the polynucleotide sequences or subsequences is involved in a receptor-ligand reaction on which detection is based.

33. A method for screening an anti-tumor agent comprising the method according to claim 26 wherein the polynucleotide sample has been treated with an anti-tumor agent to be screened.

34. A polynucleotide library according to claim 1 wherein said sequences or subsequences correspond substantially to any combination of at least one polynucleotide sequence selected among those included in each one of predefined polynucleotide sequence sets comprising:
SET 1: (SEQ ID No: 1; SEQ ID No: 2); SET 2: (SEQ ID No: 3; SEQ ID No: 4); SET 3: (SEQ ID No: 5; SEQ ID No: 6); SET 4: (SEQ ID No: 7;SEQ ID No: 8); SET 5: (SEQ ID No: 9; SEQ ID No: 10); SET 6: (SEQ ID No: 1: SEQ ID No: 12); SET 7: (SEQ ID No: 13; SEQ ID No: 14;SEQ ID No: 15); SET 8: (SEQ ID No: 16); SET 9: (SEQ ID No: 17; SEQ ID No: 18; SEQ ID No: 19); SET 10: (SEQ ID No: 20; SEQ ID No: 21); SET 11: (SEQ ID No: 22; SEQ ID No: 23; SEQ ID No: 24); SET 12: (SEQ ID No: 25; SEQ ID No: 26); SET 13: (SEQ ID No: 27; SEQ ID No: 28; SEQ ID No: 29); SET 14: (SEQ ID No: 30; SEQ ID No: 31); SET 15: (SEQ ID No: 32; SEQ ID No: 33; SEQ ID No: 34); SET 16: (SEQ ID No: 35); SET 17: (SEQ ID No: 36; SEQ ID No: 37; SEQ ID No: 38); SET 18: (SEQ ID No: 39; SEQ ID No: 40; SEQ ID No: 41); SET 19: (SEQ ID No: 42; SEQ ID No: 43); SET 20: (SEQ ID No: 44; SEQ ID No: 45); SET 21: (SEQ ID No: 46; SEQ ID No: 47); SET 22: (SEQ ID No: 48; SEQ ID No: 49; SEQ ID No: 50); SET 23: (SEQ ID No: 51; SEQ ID No: 52; SEQ ID No: 53); SET 24: (SEQ ID No: 54; SEQ ID No: 55; SEQ ID No: 56); SET 25: (SEQ ID No: 57; SEQ ID No: 58); SET 26: (SEQ ID No: 59; SEQ ID No: 60; SEQ ID No: 61); SET 27: (SEQ ID No: 62; SEQ ID No: 63; SEQ ID No: 64); SET 28 : (SEQ ID No: 65; SEQ ID No: 66; SEQ ID No: 67); SET 29 : (SEQ ID No: 68; SEQ ID No: 69; SEQ ID No: 70); SET 30: (SEQ ID No: 71; SEQ ID No: 72); SET 31: (SEQ ID No: 73; SEQ ID No: 74; SEQ ID No: 75); SET 32: (SEQ ID No: 76; SEQ ID No: 77; SEQ ID No: 78); SET 33: (SEQ ID No: 79; SEQ ID No: 80; SEQ ID No: 81); SET 34: (SEQ ID No: 82; SEQ ID No: 83); SET 35: (SEQ ID No: 84; SEQ ID No: 85); SET 36: (SEQ ID No: 86; SEQ ID No: 87); SET 37: (SEQ ID No: 88; SEQ ID No: 89; SEQ ID No: 90); SET 38: (SEQ ID No: 91; SEQ ID No: 92; SEQ ID No: 93); SET 39: (SEQ ID No: 94; SEQ ID No: 95; SEQ ID No: 96); SET 40: (SEQ ID No: 97; SEQ ID No: 98; SEQ ID No: 99) ; SET 41: (SEQ ID No: 100; SEQ ID No: 101; SEQ ID No: 78); SET 42: (SEQ ID No: 102; SEQ ID No: 103); SET 43: (SEQ ID No: 104; SEQ ID No: 105); SET 44: (SEQ ID No: 106; SEQ ID No: 107; SEQ ID No: 108); SET 45: (SEQ ID No: 109; SEQ ID No: 110); SET 46: (SEQ ID No: 111; SEQ ID No: 112; SEQ ID No: 113); SET 47: (SEQ ID No: 114); SET 48: (SEQ ID No: 115; SEQ ID No: 116; SEQ ID No: 117); SET 49: (SEQ ID No: 118; SEQ ID No: 119); SET 50: (SEQ ID No: 120; SEQ ID No: 121); SET 51: (SEQ ID No: 122; SEQ ID No: 78); SET 52: (SEQ ID No: 123; SEQ ID No: 124; SEQ ID No: 125); SET 53: (SEQ ID No: 126; SEQ ID No: 127; SEQ ID No: 128); SET 54: (SEQ ID No: 129; SEQ ID No: 130); SET 55: (SEQ ID No: 131; SEQ ID No: 132); SET 56: (SEQ ID No: 133; SEQ ID No: 134); SET 57: (SEQ ID No: 135; SEQ ID No: 136; SEQ ID No: 137); SET 58: (SEQ ID No: 138; SEQ ID No: 139; SEQ ID No: 140); SET 59: (SEQ ID No: 141; SEQ ID No: 142; SEQ ID No: 143); SET 60: (SEQ ID No: 144; SEQ ID No: 145; SEQ ID No: 146) SET 61: (SEQ ID No: 147; SEQ ID No: 148; SEQ ID No: 149); SET 62: (SEQ ID No: 150; SEQ ID No: 151; SEQ ID No: 152); SET 63: (SEQ ID No: 153; SEQ ID No: 154; SEQ ID No: 155); SET 64: (SEQ ID No: 156; SEQ ID No: 157; SEQ ID No: 158); SET 65: (SEQ ID No: 159; SEQ ID No: 160; SEQ ID No: 161); SET 66 : (SEQ ID No: 162; SEQ ID No: 163); SET 67: (SEQ ID No: 164; SEQ ID No: 165); SET 68: (SEQ ID No: 166; SEQ ID No: 167; SEQ ID No: 152) ;SET 69: (SEQ ID No: 168; SEQ ID No: 169; SEQ ID No: 170); SET 70: (SEQ ID No: 171; SEQ ID No: 172); SET 71: (SEQ ID No: 173; SEQ ID No: 174; SEQ ID No: 175); SET 72: (SEQ ID No: 176; SEQ ID No: 177); SET 73 : (SEQ ID No: 178; SEQ ID No: 179); SET 74: (SEQ ID No: 180; SEQ ID No: 181; SEQ ID No: 182); SET 75 : (SEQ ID No: 183; SEQ ID No: 184); SET 76: (SEQ ID No: 185); SET 77: (SEQ ID No: 186); SET 78: (SEQ ID No: 187; SEQ ID No: 188); SET 79: (SEQ ID No: 189; SEQ ID No: 190; SEQ ID No: 191); SET 80: (SEQ ID No: 192; SEQ ID No: 193) ;SET 81: (SEQ ID No: 194; SEQ ID No: 195); SET 82: (SEQ ID No: 196; SEQ ID No: 197; SEQ ID No: 198); SET 83: (SEQ ID No: 199; SEQ ID No: 200); SET 84: (SEQ ID No: 201; SEQ ID No: 202; SEQ ID No: 203); SET 85: (SEQ ID No: 204; SEQ ID No: 205); SET 86: (SEQ ID No: 206; SEQ ID No: 207); SET 87: (SEQ ID No: 208; SEQ ID No: 209); SET 88: (SEQ ID No: 210; SEQ ID No: 211); SET 89: (SEQ ID No: 212; SEQ ID No: 213); SET 90: (SEQ ID No: 214; SEQ ID No: 215); SET 91: (SEQ ID No: 216; SEQ ID No: 217); SET 92: (SEQ ID No: 218; SEQ ID No: 219; SEQ ID No: 220); SET 93 : (SEQ ID No: 221; SEQ ID No: 222); SET 94: (SEQ ID No: 223; SEQ ID No: 224; SEQ ID No: 225); SET 95: (SEQ ID No: 226; SEQ ID No: 227); SET 96: (SEQ ID No: 228; SEQ ID No: 229); SET 97: (SEQ ID No: 230; SEQ ID No: 231; SEQ ID No: 232); SET 98: (SEQ ID No: 233; SEQ ID No: 234); SET 99: (SEQ ID No: 235; SEQ ID No: 236; SEQ ID No: 237) ;SET 100 : (SEQ ID No: 238; SEQ ID No: 239); SET 101: (SEQ ID No: 240; SEQ ID No: 241); SET 102: (SEQ ID No: 242; SEQ ID No: 243; SEQ ID No: 244); SET 103: (SEQ ID No: 245; SEQ ID No: 246; SEQ ID No: 247); SET 104: (SEQ ID No: 248; SEQ ID No: 249); SET 105 :(SEQ ID No: 250; SEQ ID No: 251; SEQ ID No: 252); SET 106: (SEQ ID No: 253; SEQ ID No: 254); SET 107: (SEQ ID No: 255; SEQ ID No: 256); SET 108: (SEQ ID No: 257; SEQ ID No: 258); SET 109 (SEQ ID No: 259; SEQ ID No: 260; SEQ ID No: 261); SET 110: (SEQ ID No: 262; SEQ ID No: 200); SET 11: (SEQ ID No: 263; SEQ ID No: 264); SET 112: (SEQ ID No: 265; SEQ ID No: 266) SET 113 :(SEQ ID No: 267; SEQ ID No: 268); SET 114: (SEQ ID No: 269; SEQ ID No: 270); SET 115: (SEQ ID No: 271; SEQ ID No: 272); SET 116: (SEQ ID No: 273; SEQ ID No: 274); SET 117: (SEQ ID No: 275; SEQ ID No: 276); SET 118: (SEQ ID No: 277; SEQ ID No: 278); SET 119 :(SEQ ID No: 279; SEQ ID No: 280; SEQ ID No: 281); SET 120: (SEQ ID No: 282; SEQ ID No: 283; SEQ ID No: 276); SET 121: (SEQ ID No: 284; SEQ ID No: 285); SET 122: (SEQ ID No: 286; SEQ ID No: 287; SEQ ID No: 288); SET 123: (SEQ ID No: 289; SEQ ID No: 290); SET 124: (SEQ ID No: 291; SEQ ID No: 292); SET 125 : (SEQ ID No: 293; SEQ ID No: 294; SEQ ID No: 295); SET 126: (SEQ ID No: 296; SEQ ID No: 297); SET 127: (SEQ ID No: 298; SEQ ID No: 299; SEQ ID No: 300); SET 128: (SEQ ID No: 301; SEQ ID No: 302; SEQ ID No: 288); SET 129: (SEQ ID No: 303; SEQ ID No: 304); SET 130: (SEQ ID No: 305; SEQ ID No: 306; SEQ ID No: 307); SET 131: (SEQ ID No: 308; SEQ ID No: 309; SEQ ID No: 310); SET 132: (SEQ ID No: 311; SEQ ID No: 312; SEQ ID No: 313); SET 133: (SEQ ID No: 314; SEQ ID No: 315; SEQ ID No: 316); SET 134: (SEQ ID No: 317; SEQ ID No: 318); SET 135 : (SEQ ID No: 319; SEQ ID No: 320; SEQ ID No: 321); SET 136: (SEQ ID No: 322; SEQ ID No: 323); SET 137: (SEQ ID No: 324; SEQ ID No: 325) ; SET 138: (SEQ ID No: 326; SEQ ID No: 327; SEQ ID No: 328); SET 139: (SEQ ID No: 329; SEQ ID No: 330); SET 140: (SEQ ID No: 331;SEQ ID No: 332;SEQ ID No: 333); SET 141: (SEQ ID No: 334; SEQ ID No: 335; SEQ ID No: 336); SET 142: (SEQ ID No: 337; SEQ ID No: 338; SEQ ID No: 117); SET 143: (SEQ ID No: 339; SEQ ID No: 340;SEQ ID No: 341); SET 144: (SEQ ID No: 342; SEQ ID No: 343; SEQ ID No: 344); SET 145 : (SEQ ID No: 345; SEQ ID No: 346); SET 146: (SEQ ID No: 347; SEQ ID No: 348; SEQ ID No: 349); SET 147: (SEQ ID No: 350; SEQ ID No: 351); SET 148: (SEQ ID No: 352; SEQ ID No: 353); SET 149: (SEQ ID No: 354; SEQ ID No: 355); SET 150: (SEQ ID No: 356; SEQ ID No: 357); SET 151: (SEQ ID No: 358; SEQ ID No: 359; SEQ ID No: 360); SET 152: (SEQ ID No: 361; SEQ ID No: 31); SET 153: (SEQ ID No: 362; SEQ ID No: 363; SEQ ID No: 364); SET 154: (SEQ ID No: 365; SEQ ID No: 366; SEQ ID No: 367); SET 155: (SEQ ID No: 368; SEQ ID No: 369; SEQ ID No: 300); SET 156: (SEQ ID No: 370; SEQ ID No: 371); SET 157: (SEQ ID No: 372; SEQ ID No: 373; SEQ ID No: 108); SET 158: (SEQ ID No: 374; SEQ ID No: 375; SEQ ID No: 376); SET 159: (SEQ ID No: 377; SEQ ID No: 378; SEQ ID No: 379); SET 160: (SEQ ID No: 380; SEQ ID No: 381); SET 161: (SEQ ID No: 382; SEQ ID No: 383; SEQ ID No: 384); SET 162: (SEQ ID No: 385; SEQ ID No: 386; SEQ ID No: 387); SET 163: (SEQ ID No: 388; SEQ ID No: 389; SEQ ID No: 390); SET 164: (SEQ ID No: 391; SEQ ID No: 392; SEQ ID No: 393); SET 165: (SEQ ID No: 394; SEQ ID No: 395); SET 166: (SEQ ID No: 396; SEQ ID No: 397; SEQ ID No: 398); SET 167: (SEQ ID No: 399; SEQ ID No: 400; SEQ ID No: 117) ;SET 168: (SEQ ID No: 401); SET 169: (SEQ ID No: 402; SEQ ID No: 403); SET 170: (SEQ ID No: 404; SEQ ID No: 405; SEQ ID No: 318); SET 171: (SEQ ID No: 406; SEQ ID No: 407; SEQ ID No: 408); SET 172: (SEQ ID No: 409; SEQ ID No: 410; SEQ ID No: 411); SET 173: (SEQ ID No: 412; SEQ ID No: 413); SET 174: (SEQ ID No: 414; SEQ ID No: 415; SEQ ID No: 416); SET 175: (SEQ ID No: 417; SEQ ID No: 418; SEQ ID No: 419); SET 176: (SEQ ID No: 420; SEQ ID No: 421; SEQ ID No: 422); SET 177: (SEQ ID No: 423; SEQ ID No: 424; SEQ ID No: 425); SET 178: (SEQ ID No: 426; SEQ ID No: 427; SEQ ID No: 428); SET 179: (SEQ ID No: 429; SEQ ID No: 408); SET 180: (SEQ ID No: 430); SET 181: (SEQ ID No: 431); SET 182: (SEQ ID No: 432); SET 183: (SEQ ID No: 433; SEQ ID No: 434); SET 184: (SEQ ID No: 435; SEQ ID No: 436); SET 185: (SEQ ID No: 437); SET 186: (SEQ ID No: 438; SEQ ID No: 439); SET 187: (SEQ ID No: 440; SEQ ID No: 441); SET 188: (SEQ ID No: 442); SET 189: (SEQ ID No: 443); SET 190: (SEQ ID No: 444); SET 191: (SEQ ID No: 329; SEQ ID No: 330; SEQ ID No: 345); SET 192: (SEQ ID No: 446; SEQ ID No: 447); SET 193 : (SEQ ID No: 380; SEQ ID No: 381; SEQ ID No: 448); SET 194: (SEQ ID No: 449); SET 195: (SEQ ID No: 271; SEQ ID No: 272; SEQ ID No: 450); SET 196: (SEQ ID No: 84; SEQ ID No: 85; SEQ ID No: 451); SET 197: (SEQ ID No: 452); SET 198 : (SEQ ID No: 453); SET 199 :(SEQ ID No: 454); SET 200: (SEQ ID No: 183; SEQ ID NO: 184; SEQ ID No: 455); SET 201: (SEQ ID No: 456); SET 202: (SEQ ID No: 402; SEQ ID No: 403; SEQ ID No: 457); SET 203: (SEQ ID No: 458); SET 204: (SEQ ID No: 459); SET 205: (SEQ ID No: 460); SET 206: (SEQ ID No: 461); SET 207: (SEQ ID No: 462); SET 208: (SEQ ID No: 463); SET 209: (SEQ ID No: 464); SET 210: (SEQ ID No: 465); SET 211: (SEQ ID No: 466); SET 212: (SEQ ID No: 467); SET 213: (SEQ ID No: 468).

35. The library of claim 1 wherein the pool of polynucleotide sequences or subsequences correspond substantially to any combination of at least one polynucleotide sequence selected among those included in each one of predefined polynucleotide sequence sets comprising:
SET 1: (SEQ ID No: l; SEQ ID No: 2); SET 4: (SEQ ID No: 7 ; SEQ ID No: 8); SET 18: (SEQ ID No: 39 ; SEQ ID No: 40 ; SEQ ID No: 41); SET 21: (SEQ ID No: 46 ; SEQ ID No: 47); SET 24: (SEQ ID No: 54; SEQ ID No: 55; SEQ ID No: 56); SET 32: (SEQ ID No: 76; SEQ ID No: 77 ; SEQ ID No: 78); SET 38: (SEQ ID No: 91; SEQ ID No: 92 ; SEQ ID No: 93); SET 48: (SEQ ID No: 115; SEQ ID No: 116; SEQ ID No: 117); SET 53: (SEQ ID No: 126; SEQ ID No: 127; SEQ ID No: 128); SET 58: (SEQ ID No: 138 ; SEQ ID No: 139 ; SEQ ID No: 140); SET 59: (SEQ ID No: 141; SEQ ID No: 142 ; SEQ ID No: 143); SET 61: (SEQ ID No: 147 ; SEQ ID No: 148; SEQ ID No: 149); SET 64: (SEQ ID No: 156 ; SEQ ID No: 157 ; SEQ ID No: 158); SET 66: (SEQ ID No: 162 ; SEQ ID No: 163); SET 69: (SEQ ID No: 168 ; SEQ ID No: 169; SEQ ID No: 170); SET 73: (SEQ ID No: 178; SEQ ID No: 179); SET 85: (SEQ ID No: 204; SEQ ID No: 205); SET 88: (SEQ ID No: 210; SEQ ID No: 211); SET 91: (SEQ ID No: 216; SEQ ID No: 217); SET 97: (SEQ ID No: 230; SEQ ID No: 231; SEQ ID No: 232); SET 104: (SEQ ID No: 248; SEQ ID No: 249); SET 105: (SEQ ID No: 250; SEQ ID No: 251; SEQ ID No: 252); SET 112: (SEQ ID No: 265 ; SEQ ID No: 266); SET 113: (SEQ ID No: 267 ; SEQ ID No: 268); SET 115 ; (SEQ ID No: 271; SEQ ID No: 272); SET 131: (SEQ ID No: 308 ; SEQ ID No: 309; SEQ ID No: 310); SET 132: (SEQ ID No: 311; SEQ ID No: 312; SEQ ID No: 313); SET 134: (SEQ ID No: 317; SEQ ID No: 318); SET 137: (SEQ ID No: 324; SEQ ID No: 325); SET 145: (SEQ ID No: 345 ; SEQ ID No: 346); SET 147: (SEQ ID No: 350; SEQ ID No: 351); SET 155: (SEQ ID No: 368 ; SEQ ID No: 369 ; SEQ ID No: 300); SET 175: (SEQ ID No: 417; SEQ ID No: 418; SEQ ID No: 419); SET 180: (SEQ ID No: 430) SET 181: (SEQ ID No: 431); SET 182: (SEQ ID No: 432); SET 185: (SEQ ID No: 437); SET 187: (SEQ ID No: 440; SEQ ID No: 441),
wherein said sequences are useful in differentiating a normal cell from a cancer cell.

36. A polynucleotide library according to claim 1 wherein the pool of polynucleotide sequences or subsequences correspond substantially to any combination of at least one polynucleotide sequence selected among those included in each one of predefined polynucleotide sequence sets comprising:
SET 32: (SEQ ID No: 76; SEQ ID No: 77; SEQ ID No: 78); SET 73: (SEQ ID No: 178; SEQ ID No: 179); SET 131: (SEQ ID No: 308; SEQ ID No: 309 ; SEQ ID No: 310); SET 145: (SEQ ID No: 345; SEQ ID No: 346) and SET 181: (SEQ ID No: 431).
and of at least one polynucleotide sequence selected among those included in each one of predefined polynucleotide sequences sets comprising:
SET 38 : (SEQ ID No: 91; SEQ ID No: 92 ; SEQ ID No: 93); SET 58 : (SEQ ID No: 138 SEQ ID No: 139 ; SEQ ID No: 140); SET 61: (SEQ ID No: 147 ; SEQ ID No: 148; SEQ ID No: 149); SET 69: (SEQ ID No: 168 ; SEQ ID No: 169 ; SEQ ID No: 170) and SET 182: (SEQ ID No: 432).

37. The library of claim 1 wherein the pool of polynucleotide sequences or subsequences correspond substantially to any combination of at least one polynucleotide sequence selected among those included in each one of predefined polynucleotide sequence sets comprising:
SET 11: (SEQ ID No: 22 ; SEQ ID No: 23 ; SEQ ID No: 24); SET 26: (SEQ ID No: 59; SEQ ID No: 60;SEQ ID No: 61);SET 32:(SEQ ID No: 76;SEQ ID No: 77;SEQ ID No: 78);SET 34: (SEQ ID No: 82 ; SEQ ID No: 83); SET 40: (SEQ ID No: 97 ; SEQ ID No: 98 ; SEQ ID No: 99); SET 57: (SEQ ID No: 135; SEQ ID No: 136; SEQ ID No: 137); SET 64: (SEQ ID No: 156; SEQ ID No: 157; SEQ ID No: 158); SET 107: (SEQ ID No: 255; SEQ ID No: 256); SET 119: (SEQ ID No: 279; SEQ ID No: 280; SEQ ID No: 281); SET 136: (SEQ ID No: 322; SEQ ID No: 323); SET 140: (SEQ ID No: 331; SEQ ID No: 332; SEQ ID No: 333); SET 141: (SEQ ID No: 334; SEQ ID No: 335; SEQ ID No: 336); SET 145: (SEQ ID No: 345; SEQ ID No: 346); SET 148: (SEQ ID No: 352; SEQ ID No: 353); SET 149: (SEQ ID No: 354; SEQ ID No: 355); SET 162: (SEQ ID No: 385; SEQ ID No: 386; SEQ ID No: 387); SET 165: (SEQ ID No: 394; SEQ ID No: 395); SET 169 (SEQ ID No: 402; SEQ ID No: 403); SET 174: (SEQ ID No: 414; SEQ ID No: 415 ; SEQ ID No: 416) and SET 188: (SEQ ID No: 442),
wherein said sequences are useful in detecting a hormone-sensitive tumor cell.

38. The library of claim 37 wherein the pool of polynucleotide sequences or subsequences correspond substantially to any combination of at least one polynucleotide sequence selected among those included in each one of predefined polynucleotide sequence sets comprising:
SET 32: (SEQ ID No: 76; SEQ ID No: 77; SEQ ID No: 78); SET 136: (SEQ ID No: 322; SEQ ID No: 323); SET 145 : (SEQ ID No: 345 ; SEQ ID No: 346); SET 149: (SEQ ID No: 354; SEQ ID No: 355) and SET 169: (SEQ ID No: 402; SEQ ID No: 403)
and of at least one polynucleotide sequence selected among those included in each one of predefined polynucleotide sequence sets comprising:
SET 11: (SEQ ID No: 22 ; SEQ ID No: 23; SEQ ID No: 24); SET 40: (SEQ ID No: 97; SEQ ID No: 98; SEQ ID No: 99); SET 57: (SEQ ID No: 135 ; SEQ ID No: 136; SEQ ID No: 137); SET 119: (SEQ ID No: 279; SEQ ID No: 280; SEQ ID No: 281) and SET 174: (SEQ ID No: 414; SEQ ID No: 415 ; SEQ ID No: 416).

39. The library of claim 1 wherein the pool of polynucleotide sequences or subsequences correspond substantially to any combination of at least one polynucleotide sequence selected among those included in each one of predefined polynucleotide sequence sets comprising:
SET 8: (SEQ ID No: 16); SET ll: (SEQ ID No: 22; SEQ ID No: 23; SEQ ID No: 24); SET 18: (SEQ ID No: 39; SEQ ID No: 40; SEQ ID No: 41); SET 25: (SEQ ID No: 57; SEQ ID No: 58); SET 32: (SEQ ID No: 76; SEQ ID No: 77; SEQ ID No: 78); SET 34: (SEQ ID No: 82; SEQ ID No: 83); SET 40: (SEQ ID No: 97; SEQ ID No: 98; SEQ ID No: 99); SET 49: (SEQ ID No: 118; SEQ ID No: 119);SET 57:(SEQ ID No: 135;SEQ ID No: 136;SEQ ID No: 137) ;SET 91: (SEQ ID No: 216; SEQ ID No: 217); SET 100 : (SEQ ID No: 238 ; SEQ ID No: 239); SET 105 :(SEQ ID No: 250; SEQ ID No: 251: SEQ ID No: 252); SET 136: (SEQ ID No: 322 ; SEQ ID No: 323); SET 138 : (SEQ ID No: 326 ; SEQ ID No: 327; SEQ ID No: 328); SET 139 : (SEQ ID No: 329 ; SEQ ID No: 330); SET 141: (SEQ ID No: 334 ; SEQ ID No: 335 ; SEQ ID No: 336); SET 158 :(SEQ ID No: 374; SEQ ID No: 375 ; SEQ ID No: 376); SET 169: (SEQ ID No: 402; SEQ ID No: 403); SET 180: (SEQ ID No: 430) and SET 186: (SEQ ID No: 438 ; SEQ ID No: 439),
wherein said sequences are useful in differentiating a tumor in which a lymph node has been invaded by a tumor cell from a tumor in which a lymph node has not been invaded by a tumor cell.

40. The library of claim 39 wherein the pool of polynucleotide sequences or subsequences correspond substantially to any combination of at least one polynucleotide sequence selected among those included in each one of predefined polynucleotide sequence sets comprising:
SET 18 : (SEQ ID No: 39 ; SEQ ID No: 40 ; SEQ ID No: 41); SET 32: (SEQ ID No: 76 SEQ ID No: 77; SEQ ID No: 78); SET 57: (SEQ ID No: 135 ; SEQ ID No: 136; SEQ ID No: 137); SET 91: (SEQ ID No: 216; SEQ ID No: 217) and SET 105 (SEQ ID No: 250; SEQ ID No: 251; SEQ ID No: 252)
and of at least one polynucleotide sequence selected among those included in each one of predefined polynucleotide sequence sets comprising:
SET 11: (SEQ ID No: 22; SEQ ID No: 23; SEQ ID No: 24); SET 40: (SEQ ID No: 97; SEQ ID No: 98 SEQ ID No: 99);SET 49: (SEQ ID No: 118; SEQ ID No: 119);SET 100: (SEQ ID No: 238; SEQ ID No: 239) and SET 141: (SEQ ID No: 334; SEQ ID No: 335 ; SEQ ID No: 336).

41. The library of claims 1 or 2 wherein the pool of polynucleotide sequences or subsequences correspond substantially to any combination of at least one polynucleotide sequence selected among those included in each one of predefined polynucleotide sequence sets comprising:
SET 1: (SEQ ID No: 22; SEQ ID No: 23; SEQ ID No: 24); SET 22: (SEQ ID No: 48; SEQ ID No: 49 ; SEQ ID No: 50); SET 23 :(SEQ ID No: 51; SEQ ID No: 52 ; SEQ ID No: 53); SET 26: (SEQ ID No: 59; SEQ ID No: 60; SEQ ID No: 61); SET 28: (SEQ ID No: 65 ; SEQ ID No: 66 ; SEQ ID No: 67); SET 31: (SEQ ID No: 73; SEQ ID No: 74; SEQ ID No: 75); SET 32: (SEQ ID No: 76; SEQ ID No: 77 ; SEQ ID No: 78); SET 34: (SEQ ID No: 82 ; SEQ ID No: 83); SET 49: (SEQ ID No: 118 ; SEQ ID No: 119); SET 57: (SEQ ID No: 135 ; SEQ ID No: 136 ; SEQ ID No: 137); SET 64: (SEQ ID No: 156; SEQ ID No: 157 ; SEQ ID No: 158); SET 73: (SEQ ID No: 178; SEQ ID No: 179); SET 77: (SEQ ID No: 186); SET 81 : (SEQ ID No: 194; SEQ ID No: 195); SET 95: (SEQ ID No: 226 ; SEQ ID No: 227); SET 131: (SEQ ID No: 308 ; SEQ ID No: 309 ;SEQ ID No: 310); SET 138: (SEQ ID No: 326; SEQ ID No: 327 ; SEQ ID No: 328); SET 140: (SEQ ID No: 331; SEQ ID No: 332; SEQ ID No: 333); SET 149: (SEQ ID No: 354; SEQ ID No: 355); SET 162: (SEQ ID No: 385; SEQ ID No: 386 ; SEQ ID No: 387); SET 164: (SEQ ID No: 391; SEQ ID No: 392; SEQ ID No: 393); SET 165: (SEQ ID No: 394; SEQ ID No: 395) and SET 183: (SEQ ID No: 433; SEQ ID No: 434).
wherein said sequences are usefal in differentiating anthracycline-sensitive tumors from anthracycline-insensitive tumors.

42. A library according to claim 41 wherein the pool of polynucleotide sequences or subsequences correspond substantially to any combination of at least one polynucleotide sequence selected among those included in each one of predefined polynucleotide sequence sets consisting of
SET 32: (SEQ ID No: 76; SEQ ID No: 77, SEQ ID No: 78); SET 36: (SEQ ID No: 322; SEQ ID No: 323); SET 145: (SEQ ID No: 345; SEQ ID No: 346); SET 149: (SEQ ID No: 354; SEQ ID No: 355); SET 169: (SEQ ID No: 402; SEQ ID No: 403)
and of at least one polynucleotide sequence sets consisting of:
SET 11: (SEQ ID No: 22; SEQ ID No: 23; SEQ ID No: 24); SET 40: (SEQ ID No: 97; SEQ ID No: 98; SEQ ID No: 99); SET 57: (SEQ ID No: 135; SEQ ID No: 136; SEQ ID No: 137); SET 119: (SEQ ID No: 279; SEQ ID No: 280; SEQ ID No: 281); SET 174: (SEQ ID No: 414; SEQ ID No: 415; SEQ ID No: 416).

43. The library of claim 1 wherein the pool of polynucleotide sequences or subsequences correspond substantially to any combination of at least one polynucleotide sequence selected among those included in each one of predefined polynucleotide sequences sets comprising:
SET 14 (SEQ ID No: 30; SEQ ID No: 31); SET 23 (SEQ ID No: 51; SEQ ID No: 52; SEQ ID No: 53); SET 25 (SEQ ID No: 57; SEQ ID No: 58); SET 27 (SEQ ID No: 62; SEQ ID No: 63; SEQ ID No: 64); SET 28 (SEQ ID No: 65; SEQ ID No: 66; SEQ ID No: 67); SET 32 (SEQ ID No: 76; SEQ ID No: 77; SEQ ID No: 78); SET 39 (SEQ ID No: 94; SEQ ID No: 95; SEQ ID No: 96); SET 41 (SEQ ID No: 100; SEQ ID No: 101; SEQ ID No: 78); SET 44 (SEQ ID No: 106; SEQ ID No: 107; SEQ ID No: 108); SET 48 (SEQ ID No: 115; SEQ ID No: 116; SEQ ID No: 117); SET 51 (SEQ ID No: 122; SEQ ID No: 78); SET 64 (SEQ ID No: 156; SEQ ID No: 157; SEQ ID No: 158); SET 81 (SEQ ID No: 194; SEQ ID No: 195); SET 83 (SEQ ID No: 199; SEQ ID No: 200); SET 91 (SEQ ID No: 216; SEQ ID No: 217); SET 96 (SEQ ID No: 228; SEQ ID No: 229); SET 99 (SEQ ID No: 235; SEQ ID No: 236; SEQ ID No: 237); SET 108 (SEQ ID No: 257; SEQ ID No: 258); SET 110 (SEQ ID No: 262; SEQ ID No: 200); SET 116 (SEQ ID No: 273; SEQ ID No: 274); SET 117 (SEQ ID No: 275; SEQ ID No: 276); SET 118 (SEQ ID No: 277; SEQ ID No: 278); SET 120 (SEQ ID No: 282; SEQ ID No: 283; SEQ ID No: 276); SET 126 (SEQ ID No: 296; SEQ ID No: 297;); SET 142 (SEQ ID No: 337; SEQ ID No: 338; SEQ ID No: 117); SET 144 (SEQ ID No: 342; SEQ ID No: 343; SEQ ID No: 344); SET 149 (SEQ ID No: 354; SEQ ID No: 355); SET 152 (SEQ ID No: 361; SEQ ID No: 31); SET 153 (SEQ ID No: 362; SEQ ID No: 363; SEQ ID No: 364) SET 154 (SEQ ID No: 365; SEQ ID No: 366; SEQ ID No: 367); SET 157 (SEQ ID No: 372; SEQ ID No: 373; SEQ ID No: 108); SET 159 (SEQ ID No: 377; SEQ ID No: 378; SEQ ID No: 379); SET 162 (SEQ ID No: 385; SEQ ID No: 386; SEQ ID No: 387); SET 166 (SEQ ID No: 396; SEQ ID No: 397; SEQ ID No: 398); SET 167 (SEQ ID No: 399; SEQ ID No: 400; SEQ ID No: 117); SET 168 (SEQ ID No: 401); SET 171 (SEQ ID No: 406; SEQ ID No: 407; SEQ ID No: 408); SET 172 (SEQ ID No: 409; SEQ ID No: 410; SEQ ID No: 411); SET 173 (SEQ ID No: 412; SEQ ID No: 413); SET 176 (SEQ ID No: 420; SEQ ID No: 421; SEQ ID No: 422); SET 177 (SEQ ID No: 423; SEQ ID No: 424; SEQ ID No: 425); SET 178 (SEQ ID No: 426; SEQ ID No: 427; SEQ ID No: 428); SET 179 (SEQ ID No: 429; SEQ ID No: 408); SET 184 (SEQ ID No: 435; SEQ ID No: 436); SET 185 (SEQ ID No: 437)
wherein said sequences are useful in classifying good and poor prognosis primary breast tumors.

44. The library of claim 1 wherein the pool of polynucleotide sequences or subsequences correspond substantially to any combination of at least one polynucleotide sequence selected among those included in each one of predefined polynucleotide sequence sets comprising:
SET 23 (SEQ ID No: 51; SEQ ID No: 52; SEQ ID No: 53); SET 25 (SEQ ID No: 57 ; SEQ ID No: 58); SET 32 (SEQ ID No: 76 ; SEQ ID No: 77 ; SEQ ID No: 78); SET 41 (SEQ ID No: 100; SEQ ID No: 101; SEQ ID No: 78); SET 48 (SEQ ID No: 115; SEQ ID No: 116; SEQ ID No: 117); SET 51 (SEQ ID No: 122; SEQ ID No: 78); SET 64 (SEQ ID No: 156; SEQ ID No: 157; SEQ ID No: 158); SET 81 (SEQ ID No: 194; SEQ ID No: 195); SET 83 (SEQ ID No: 199; SEQ ID No: 200); SET 91 (SEQ ID No: 216; SEQ ID No: 217); SET 99 (SEQ ID No: 235; SEQ ID No: 236 SEQ ID No: 237); SET 110 (SEQ ID No: 262; SEQ ID No: 200); SET 116 (SEQ ID No: 273; SEQ ID No: 274); SET 142 (SEQ ID No: 337; SEQ ID No: 338; SEQ ID No: 117); SET 144 (SEQ ID No: 342; SEQ ID No: 343; SEQ ID No: 344); SET 149 (SEQ ID No: 354; SEQ ID No: 355); SET 162 (SEQ ID No: 385; SEQ ID No: 386; SEQ ID No: 387); SET 167 (SEQ ID No: 399; SEQ ID No: 400 ; SEQ ID No: 117); SET 171 (SEQ ID No: 406 ; SEQ ID No: 407; SEQ ID No: 408); SET 172 (SEQ ID No: 409 ; SEQ ID No: 410; SEQ ID No: 411); SET 173 (SEQ ID No: 412 ; SEQ ID No: 413); SET 176 (SEQ ID No: 420 ; SEQ ID No: 421; SEQ ID No: 422); SET 177 (SEQ ID No: 423 ; SEQ ID No: 424 ; SEQ ID No: 425); SET 178 (SEQ ID No: 426; SEQ ID No: 427; SEQ ID No: 428); SET 179 (SEQ ID No: 429 ; SEQ ID No: 408); SET 184 (SEQ ID No: 435 ; SEQ ID No: 436); SET 185 (SEQ ID No: 437),
and at least one polynucleotide sequence selected among those included in each one of predefined polynucleotide sequence sets comprising:
SET 14 (SEQ ID No: 30 ; SEQ ID No: 31); SET 27 (SEQ ID No: 62 ; SEQ ID No: 63; SEQ ID No: 64); SET 28 (SEQ ID No: 65 ; SEQ ID No: 66 ; SEQ ID No: 67); SET 39 (SEQ ID No: 94; SEQ ID No: 95;SEQ ID No: 96); SET44(SEQ ID No: 106;SEQ ID No: 107;SEQ ID No: 108); SET 96 (SEQ ID No: 228 ; SEQ ID No: 229); SET 108 (SEQ ID No: 257 ; SEQ ID No: 258) ; SET 117 (SEQ ID No: 275 ; SEQ ID No: 276); SET 118 (SEQ ID No: 277; SEQ ID No: 278); SET 120 (SEQ ID No: 282 ; SEQ ID No: 283 ; SEQ ID No: 276); SET 126 (SEQ ID No: 296 ; SEQ ID No: 297); SET 152 (SEQ ID No: 361; SEQ ID No: 31); SET 153 (SEQ ID No: 362 ; SEQ ID No: 363; SEQ ID No: 364); SET 154 (SEQ ID No: 365 ; SEQ ID No: 366; SEQ ID No: 367); SET 157 (SEQ ID No: 372 ; SEQ ID No: 373; SEQ ID No: 108); SET 159 (SEQ ID No: 377 ; SEQ ID No: 378; SEQ ID No: 379); SET 166 (SEQ ID No: 396; SEQ ID No: 397; SEQ ID No: 398); SET 168 (SEQ ID No: 401),
wherein the combination of overexpression of the genes identified by said first group of cluster sequences with the underexpression of the genes identified by said second group of cluster sequences are useful in classifying good and poor prognosis primary breast tumors.

45. The polynucleotide library of claim 1 wherein said tumor cells are breast tumor cells.

46. The polynucleotide library of claim 1 wherein said polynucleotides are immobilized on a solid support in order to form a polynucleotide array.

47. The polynucleotide library of claim 46 wherein the support is selected from the group consisting of a nylon membrane, nitrocellulose membrane, glass slide, glass beads, membranes on glass support or a silicon chip.

48. A polynucleotide array useful for prognosis or diagnosis of a tumor comprising an immobilized polynucleotide library according to claim 1 or 34.

49. A polynucleotide array useful to differentiate a normal cell from a cancer cell comprising any combination of immobilized polynucleotide sequence sets according to claim 35.

50. A polynucleotide array useful to differentiate a normal cell from a cancer cell comprising any combination of immobilized polynucletide sequence sets according to claim 36.

51. A polynucleotide array useful to detect a hormone-sensitive tumor cell comprising any combination of immobilized polynucleotide sequence sets according to claim 37.

52. A polynucleotide array useful to detect a hormone-sensitive tumor cell comprising any combination of immobilized polynucleotide sequence sets according to claim 38.

53. A polynucleotide array useful to differentiate a tumor in which a lymph node has been invaded by a tumor cell from a tumor in which a lymph node has not been invaded by a tumor cell comprising any combination of immobilized polynucleotide sequence sets according to claim 39.

54. A polynucleotide array useful to differentiate a tumor in which a lymph node has been invaded by a tumor cell from a tumor in which a lymph node has not been invaded by a tumor cell comprising any combination of immobilized polynucleotide sequences sets according to claim 40.

55. A polynucleotide array useful to differentiate anthracycline-sensitive tumors from anthracycline-insensitive tumors comprising any combination of immobilized polynucleotide sequence sets according to claim 41.

56. A polynucleotide array useful to classify good and poor prognosis primary breast tumors comprising any combination of immobilized polynucleotide sequence sets according to claim 42.

57. A polynucleotide array useful to classify good and poor prognosis primary breast tumors comprising any combination of immobilized polynucleotide sequence sets according to claim 43.

58. A polynucleotide array useful to classify good and poor prognosis primary breast tumors comprising any combination of polynucleotide sequence sets according to claim 44.

59. A method for detecting differentially expressed polynucleotide sequences which are correlated with a cancer, said method comprising:
obtaining a polynucleotide sample from a patient;
reacting said polynucleotide sample with a probe immobilized on a solid support wherein said probe comprises any of the polynucleotide sequences of the polynucleotide library of claim 1 or an expression product encoded by any of the polynucleotide sequences of the polynucleotide library of claim 1;
detecting a polynucleotide sample reaction product.

60. The method of claim 58 wherein said polynucleotide sample is labeled before said reacting step.

61. The method of claim 60 wherein the label of the polynucleotide sample is selected from the group consisting of radioactive, colorimetric, enzymatic, molecular amplification, bioluminescent or fluorescent labels.

62. The method of claim 59 furither comprising obtaining a control polynucleotide sample, reacting said control sample with said probe detecting a control sample reaction product, and comparing the amount of said polynucleotide sample reaction product to the amount of said control sample reaction product.

63. The method of claim 59 wherein RNA or mRNA is isolated from said polynucleotide sample.

64. The method of claim 63 wherein mRNA is isolated from said polynucleotide sample and cDNA is obtained by reverse transcription of said mRNA.

65. The method of claim 59 wherein said reacting step is performed by hybridizing the polynucleotide sample RNA with the probe.

66. The method of claim 59 wherein said method is used for detecting, diagnosing, staging, monitoring, predicting, preventing or treating conditions associated with cancer.

67. The method of claim 59 wherein the cancer is breast cancer.

68. The method of claim 59 wherein the product encoded by any of the polynucleotide sequences or polynucleotide sequence sets is involved in a receptor-ligand reaction on which detection is based.

69. A method for screening an anti-tumor agent comprising the method of claim 59 wherein said polynucleotide sample has been treated with an anti-tumor agent to be screened.
